(19) United States
(12) Patent Application Publication
(10) Pub. No.: US 2004/0208824 A1
(43) Pub. Date: Oct. 21, 2004

(54) Neutrokine-alpha conjugate, neutrokine-alpha complex, and uses thereof

(76) Inventors: David Parmelee, Rockville, MD (US); Ren-Hwa Yeh, Germantown, MD (US); Olga Galperina, North Potomac, MD (US); David Hilbert, Bethesda, MD (US); Craig A. Rosen, Laytonsville, MD (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/742,634

(22) Filed: Dec. 22, 2003

Related U.S. Application Data
(60) Provisional application No. 60/435,262, filed on Dec. 23, 2002.
(60) Provisional application No. 60/467,198, filed on May 2, 2003.

(51) Int. Cl. A61K 49/00; C07K 14/525

(52) U.S. Cl. 424/9.34; 530/351

(57) ABSTRACT

The present invention is directed to a Neutrokine-alpha conjugate, wherein said Neutrokine-alpha conjugate comprises a Neutrokine-alpha protein and a chelator. The present invention is also directed to a Neutrokine-alpha complex, wherein said Neutrokine-alpha complex comprises a Neutrokine-alpha conjugate and a metal ion wherein said metal ion is noncovalently associated with the chelator of said conjugate. The present invention is further directed to compositions, specifically pharmaceutical and diagnostic compositions, comprising a Neutrokine-alpha conjugate or complex as described herein. Therapeutic and diagnostic uses of said conjugate, complex, and compositions are described.

This application claims the benefit of U.S. Provisional Application No. 60/467,198, filed May 2, 2003, and U.S. Provisional Application No. 60/435,262, filed Dec. 23, 2002.

### BACKGROUND OF THE INVENTION

1. Field of the Invention

The present invention is directed to a modified Neutrokine-alpha protein that can be labeled with a metal ion, such as a radionuclide, and is useful for the treatment, diagnosis, and imaging of certain conditions such as cancer and autoimmune disease.

2. Background Art

Neutrokine-alpha (also known as BLyS™ protein (B-Lymphocyte Stimulator); also known as, inter alia, TALL-1, THANK, and BAFF) is a member of the tumor necrosis factor (TNF) superfamily that induces B cell proliferation and immunoglobulin secretion (Moore et al., Science 285:260- 263 (1999)). Neutrokine-alpha also appears to be a key regulator of peripheral B cell populations in vivo due to the role Neutrokine-alpha plays in regulatingn B cell survival (Mackay et al., J. Exp. Med. 190:1697-1710 (1999); Do et al., J. Exp. Med. 192:953-964 (2000); and Hsu et al., J. Immunol. 168:5993-6 (2002)). Like other members of the TNF family, Neutrokine-alpha is a type-II membrane protein that can be cleaved at the cell surface to form a soluble protein (Mariani et al., J. Cell Biol. 137:221-229 (1997)). Neutrokine-alpha, like other members of the TNF ligand family described to date, forms biologically active trimers.

Like other members of the TNF family, Neutrokine-alpha is a ligand that interacts with several receptors. Neutrokine-alpha was initially shown to interact with TACI (trans-membrane activator and CAML interactor) and BCMA (B cell maturation antigen) (Gross et al., Nature 404:995-999 (2000)). Both receptors were found to bind APRIL as well (Marsters et al., Curr. Biol. 10:785-788 (2000); Wu et al., J. Biol. Chem. 275:35478-35485 (2000)), APRIL being the TNF ligand that has the highest degree of sequence homology with Neutrokine-alpha. Most recently, a third receptor, termed BAFF-R, has been identified. This receptor apparently does not interact with APRIL or any known TNF ligand other than Neutrokine-alpha (Thompson et al., Science 293:2108-2111 (2001)).

Neutrokine-alpha receptor expression, defined functionally by the binding of biotinylated Neutrokine-alpha to cells, is found predominantly on B cells (see, e.g., Moore et al., Science 285:260-263 (1999), although TACI has also been reported to be expressed on activated T cells (Wang et al., Nature Immunol. 2:577-8 (2001). The expression of Neutrokine-alpha is observed on normal B cells as well as on tumorous B cells, including Non-Hodgkin's lymphoma cells and Chronic Lymphocytic Leukemia cells. Furthermore, Neutrokine-alpha receptor expression is not observed in pre-B cells; rather, Neutrokine-alpha receptor expression becomes observable at the same stage in B cell development when surface Ig expression becomes apparent (see, e.g., Hsu et al., J. Immunol. 168:5993-5996 (2002).

The restricted expression profile of Neutrokine-alpha receptors on lymphoid cells, and predominantly on B cells, makes Neutrokine-alpha an attractive vehicle for targeting therapies to lymphocytes, and to B lineage cells in particular. Neutrokine-alpha thus may be used to treat and diagnose diseases and disorders of the immune system, particularly those associated with aberrant B cells numbers or function, including for example, autoimmune diseases, B cell cancers and inflammation.

Autoimmune diseases are characterized, in part, by a failure of the immune system to distinguish the body's own cells and tissues from those of pathogens. B cells that produce antibodies that recognize parts of the normal body (autoantibodies) are characteristic of many autoimmune diseases. Systemic lupus erythematosus, rheumatoid arthritis, Sjögren's syndrome, Wegener's granulomatous, myasthenia gravis, multiple sclerosis, diabetes, and some forms of asthma are all examples of autoimmune diseases that are associated with autoantibodies. Thus, an agent that inhibits the proliferation, differentiation or survival of B cells, e.g., a drug comprising Neutrokine-alpha linked to a source of radiation, could be used to treat or prevent diseases such as systemic lupus erythematosus, rheumatoid arthritis, Sjögren's syndrome, Wegener's granulomatous, myasthenia gravis, multiple sclerosis, diabetes and some forms of asthma. Such an agent would also be useful in the treatment of cancers, particularly B cell cancers or cancer cells in close proximity to B cells.

Moreover, the development of a Neutrokine-alpha protein associated with a metal ion would be useful in diagnostic processes. Such a protein conjugate could be linked to a metal ion which could be visualized using any number of known techniques including single photon emission computed tomography (SPECT), positron emission tomography (PET), and magnetic resonance imaging (MRI). A Neutrokine-alpha protein associated with a metal ion could further be used, inter alia, as an agent to inhibit the proliferation of or kill B-cells. Thus, a Neutrokine-alpha protein associated with a metal ion would be useful in the treatment of diseases caused by or involving aberrant B cell proliferation, activation or survival including, but not limited to, B cell cancers, autoimmune diseases and inflammation.

The use of a chelator conjugated to a protein for labeling said protein with radiometals allows one to label the same protein-chelator conjugate with different radiometals providing a number of choices of half-life and types of emissions for various medical applications in both diagnosis and therapy. Radiometals also offer significant advantages over iodine when used to label proteins. Radiometal labeling, for example, avoids the deleterious effects of oxidation experienced in direct iodination reactions. Labeling with metals can also overcome problems of in vivo deiodination by tumor and normal tissues, particularly when using rapidly internalized proteins.

### BRIEF SUMMARY OF THE INVENTION

The present invention provides compositions useful in the treatment and diagnosis of diseases and disorders associated with cells bearing Neutrokine-alpha receptors on their surface. As Neutrokine-alpha receptors are found predominantlty on cells of lymphoid origin, and particularly on B cells, in one embodiment the present invention provides compositions useful in the treatment and diagnosis of diseases and disorders associated with aberrant lymphocyte, and particularly B lymphocyte, number or function. In specific embodiments, the present invention provides compositions useful in the treatment and diagnosis of autoimmune diseases with a humoral autoimmune component including, nut not limited to, Systemic lupus Erythematosus, rheumatoid arthritis, Sjögren's syndrome, Wegener's granulomatous, myasthenia gravis, multiple sclerosis, diabetes, and autoimmune forms of asthma. In other specific embodiments, the present invention provides compositions useful in the treatment and diagnosis of cancers of cells bearing neutrokine-alpha receptors including B cell cancers such as Non-Hodgkins' lymphoma, chronic lymphocytic leukemia, and multiple myeloma, or cancers of other cells types that express Neutrokine-alpha receptors, (e.g., epithelial cells such as epithelial cells in the lung or colon.). In other specific embodiments, the present invention provides compositions useful in the treatment and diagnosis of cancers of cells that are in close proximity to cells expressing Neutrokine-alpha receptors, for example, cancers that have metastasized through the lymphatic system.

In one embodiment, the present invention provides a Neutrokine-alpha conjugate comprising a Neutrokine-alpha protein and a chelator. In another embodiment, the present invention provides a Neutrokine-alpha complex comprising a Neutrokine-alpha conjugate and a metal ion. In specific embodiments, a composition of the invention comprises a Neutrokine-alpha conjugate or a Neutrokine-alpha complex in an acceptable carrier.

The present invention also provides methods of preparing Neutrokine-alpha conjugates and Neutrokine-alpha complexes. In one embodiment, a Neutrokine-alpha conjugate is prepared by reacting a Neutrokine-alpha protein with a chelator. In another embodiment, a Neutrokine-alpha complex is prepared by reacting a Neutrokine-alpha conjugate with a metal ion. In another embodiment, a Neutrokine-alpha complex is prepared by reacting a Neutrokine-alpha protein with a chelator-metal ion complex.

In preferred embodiments, the Neutrokine-alpha protein component of a Neutrokine-alpha conjugate or Neutrokine-alpha complex is a protein consisting of a trimer of three identical subunits, each subunit comprising, or alternatively consisting of the protein of SEQ ID NO:3, namely amino acids 134-285 of the full-length Neutrokine-alpha protein which is shown in SEQ ID NO:2. In other embodiments, the Neutrokine-alpha protein component of a Neutrokine-alpha conjugate or Neutrokine-alpha complex, comprises or alternatively consists of the Neutrokine-alpha protein of SEQ ID NO:2, or fragments or variants thereof, e.g., amino acids 134-285 of SEQ ID NO:2.

In preferred embodiments, the chelator component of a Neutrokine-alpha conjugate or Neutrokine-alpha complex is α-(5-isothiocyanato-2-methoxyphenyl)-1,4,7,10-tetraaza-cyclododecane-1,4,7,10-tetraacetic acid. In other embodiments, the chelator of a Neutrokine-alpha conjugate or Neutrokine-alpha complex has a chemical formula according to any one of the formulas selected from the group consisting of Formula I, II, III, or IV described below.

In preferred embodiments, the metal ion component is a radionuclide that emits gamma rays, positrons, x-rays, fluorescence, beta particles, alpha particles, or auguer electrons. In other preferred embodiments, the metal ion component is a radionuclide which is an electron or neutron-capturing agent.

In preferred embodiments, the metal ion component of Neutrokine-alpha complex or chelator-metal-ion complex is yttrium-90 (⁹⁰Y). In other embodiments, the metal ion component of a Neutrokine-alpha complex or chelator-metal-ion complex is indium-111 (¹¹¹In), leuticium-177 (¹⁷⁷Lu), holmium-166 (¹⁶⁶Ho), bismuth-215 (²¹⁵Bi), and actinium-225 (²²⁵Ac).

The present invention further encompasses methods and compositions for killing cells bearing Neutrokine-alpha receptors and/or cells in close proximity to cells bearing Neutrokine-alpha receptors, comprising, or alternatively consisting of, contacting a Neutrokine-alpha conjugate and/or a Neutrokine-alpha complex of the invention with cells bearing Neutrokine-alpha receptors. In preferred embodiments, the cells bearing Neutrokine-alpha receptors are B cells.

The present invention further encompasses methods and compositions for killing cells bearing Neutrokine-alpha receptors or cells in close proximity to cells bearing a Neutrokine-alpha receptor, comprising, or alternatively consisting of, administering to an animal in which such killing is desired, a Neutrokine-alpha conjugate and/or a Neutrokine-alpha complex in an amount effective to kill cells bearing Neutrokine-alpha receptors and/or cells in close proximity to cells bearing a Neutrokine-alpha receptor. In preferred embodiments, the cells bearing Neutrokine-alpha receptors are B cells.

The present invention also provides methods of using the compositions of the invention for the diagnosis and/or treatment of B-cell cancers. Specifically contemplated is the use of Neutrokine-alpha for the diagnosis and/or treatment of non-Hodgkin's lymphoma, multiple myeloma, chronic lymphocytic leukemia (CLL), acute lymphocytic leukemia (ALL), Burkitt's lymphoma, and/or Epstein Barr-Virus (EBV) transformed B cell cancers.

The present invention also provides methods of using the compositions of the invention for the diagnosis and/or treatment of autoimmune disease. In specific embodiments, Neutrokine-alpha conjugates and/or Neutrokine-alpha complexes are used for the diagnosis and/or treatment of systemic lupus erythematosus (SLE), rheumatoid arthritis (RA), Sjögren's Syndrome, idiopathic thrombocytic purpura (ITP) hemolytic anemia, myasthenia gravis, and/or IgA nephropathy.

In specific embodiments, Neutrokine-alpha conjugates and/or Neutrokine-alpha complexes are used for the diagnosis and/or treatment of atherosclerosis.

The present invention also provides pharmaceutical kits for the preparation of a Neutrokine-alpha conjugate or Neutrokine-alpha complex.

### BRIEF DESCRIPTION OF THE DRAWINGS/FIGURES

FIG. 1 shows a proposed scheme of a method of preparing a Neutrokine-alpha complex.

FIG. 2. Effect of ³¹I-labeled Neutrokine-alpha (lot TX1) on the survival of BCL1 tumor-bearing BALB/c mice. Survival curve expressed in terms of survival probability vs. time. Day 0 is the first day of tumor cell injection. Differences among the treatment groups were analyzed using the Log Rank Test for equality. Treatment with ¹³¹I-labeled Neutrokine-alpha (LR131 in figure) at doses of either 11.9 or 15.3 mCi/kg (red and blue dotted lines, respectively) significantly prolonged survival (p=0.0162 and p=0.0052, respectively) compared with vehicle-treated controls (black solid line). In the group of mice that did not have BCL1 tumors but did receive 15.3 mCi/kg of ¹³¹I-labeled Neutrokine-alpha, 10% of the mice died (yellow dashed line).

FIG. 3. Effect of ¹³¹I-labeled Neutrokine-alpha (lot TX2) on the survival of BCL1 tumor-bearing BALB/c mice. Survival curve expressed in terms of survival probability vs. time. Day 0 is the first day of tumor cell injection. Differences among the treatment groups were analyzed using the Log Rank Test for equality. Treatment with ¹³¹1-labeled Neutrokine-alpha (LR131 in figure) at a dose of 17.5 mCi/kg (dashed line) significantly prolonged survival (p=0.0348) compared with the vehicle-treated controls (black solid line). In the group of mice that did not have BCL1 tumors but did receive ¹³¹I-labeled Neutrokine-alpha, 12.5% of the mice died (dotted line).

FIG. 4. Effect of ¹³¹I-labeled Neutrokine-alpha (lot TX3) on the survival of BCL1 tumor-bearing BALB/c mice. Survival curve expressed in terms of survival probability vs. time. Day 0 is the day the tumor cells were injected. Differences among the treatment groups were analyzed using the Log Rank Test for equality. Treatment with ¹³¹I-labeled Neutrokine-alpha (LR131 in figure) at a dose of 37.7 mCi/kg (dashed line) significantly prolonged survival (p=0.0212) compared to the vehicle-treated controls (solid line). In the group of mice that did not have BCL1 tumors but did receive ¹³¹I-labeled Neutrokine-alpha, 12.5% of the mice died (dotted line).

FIG. 5 shows a plasmid map of the pML124 vector. The sequence of this vector is shown in SEQ ID NO:14.

FIG. 6 shows a plasmid map of the pML124 vector containing the MBPss-Neutrokine-alpha fusion. The sequence of this vector is shown in SEQ ID NO:15.

### DETAILED DESCRIPTION OF THE INVENTION

The restricted expression profile of Neutrokine-alpha receptors on lymphoid cells, and predominantly on B cells, makes Neutrokine-alpha an attractive vehicle for targeting therapies to lymphocytes, and to B lineage cells in particular. To date, three receptors for Neutrokine-alpha have been identified; namely BAFF-R (Locus ID: 115650, Refseq No. NM_{—}052945, SEQ ID NO:5) TACI (Locus ID: 23495, Refseq No. NM_{—}012452, SEQ ID NO:7), and BCMA (Locus ID: 608, Refseq No. NM_{—}001192, SEQ ID NO:9). LocusID numbers refer to the NCBI Locus Link website (http://www.ncbi.nlm.nih.gov/LocusLink/list.cgi) and the “NM” Nos. are GenBank Refseq Nos. for the mRNAs encoding these receptors. Nucleotide sequences encoding these receptors are given in SEQ ID NOS: 4, 6, and 8, respectively. =p Neutrokine-alpha receptor expression, defined functionally by the binding of biotinylated Neutrokine-alpha to cells, is found predominantly on B cells (see, e.g., Moore et al., Science 285:260-263 (1999), which is hereby incorporated by reference in its entirety), although TACI has also been reported to be expressed on activated T cells (Wang et al., Nature Immunol. 2:577-8 (2001), which is hereby incorporated by reference in its entirety.) Furthermore, Neutrokine-alpha receptor expression is not observed in pre-B cells; rather, Neutrokine-alpha receptor expression becomes observable at the same stage in B cell development when surface Ig expression becomes apparent (see, e.g., Hsu et al., J. Immunol. 168:5993-5996 (2002), which is hereby incorporated by reference in its entirety).

Receptors which bind proteins comprising Neutrokine-alpha or fragments or variants thereof may also be expressed on non-hematopoietic cells. In specific embodiments, receptors which bind proteins comprising Neutrokine-alpha or fragments or variants thereof (e.g., Neutrokine-alpha heterotrimers (described below) comprising one or two APRIL monomers) are expressed on cells or cell lines of fibroblastic or epithelial lineage or having fibroblastic or epithelial morphology (e.g., NIH-3T3 fibroblasts, A549 lung carcinoma cells, or HT-29 colorectal adenocarcinoma cells).

Additionally, Neutrokine-alpha receptor expression, defined functionally by the binding of biotinylated Neutrokine-alpha to cells, has been observed on multiple myeloma, Non-Hodgkin's lymphoma, and chronic lymphocytic leukemia primary tumor explants (see, e.g., Briones et al., Experimental Hematology 30:135-141 (2002) and Novak et al., Blood 100:2973-2979 (2002), each of which is hereby incorporated by reference in its entirety) and on B lineage immortalized hematopoietic cell lines (e.g., IM-9 (ATCC CCL-159); Reh (ATCC CRL-8286); ARH-77 (ATCC CRL-1621); Raji (ATCC-CCL-86); Namalwa (CRL-1432); and RPMI 8226 (ATCC CCL-155)).

Biodistribution studies of a Neutrokine-alpha protein radiolabeled with either iodine-125 (¹²⁵I) or indium-111 (¹¹¹In) injected into BALB/c mice illustrate that a Neutrokine-alpha has high in vivo targeting specificity for lymphoid tissues such as spleen and lymph nodes (See Example 1). ¹²⁵I- and ¹¹¹In-labeled Neutrokine-alpha proteins are used as indicators of the biodistribution of therapeutic Neutrokine-alpha proteins labeled with iodine- 131 (¹³¹I) or yttrium-90 (⁹⁰Y). Furthermore, administration of the ¹³¹1-labeled form of Neutrokine-alpha to mice bearing a B cell tumor has been shown to inhibit the growth of the tumor and prolong survival (Example 2). It is usually not possible or highly unfeasible to study the effects of ⁹⁰Y-labeled Neutrokine-alpha in tumor bearing mice due to the quantity and strength of radiation involved compared to the body mass of a mouse.

Clearly, Neutrokine-alpha associated with a metal ion, such as a radionuclide, can be used as a therapy for B-cell malignancies. Such malignancies are responsive to radiation, and radiotherapy is an important part of the treatment plan for many patients with these diseases. Additionally, Neutrokine-alpha an agent comprising Neutrokine-alpha linked to a source of radiation can be used to treat other types of malignancies, (e.g. any type of cancer that metastasizes in the lymphatic system). Without being limited by this mechanism, cells in close proximity to cells bearing a Neutrokine-alpha receptor may also be killed by a Neutrokine-alpha protein linked to a source of radiation depending on the strength of the radioactive emission. Furthermore, an agent comprising Neutrokine-alpha linked to a source of radiation binds predominantly to B cells, so comparatively low doses of radiation will be effective at killing such cells.

A composition comprising Neutrokine-alpha associated with a radionuclide would also have application in the treatment of diseases associated with an aberrant number or function of cells expressing Neutrokine-alpha receptor, (e.g., lymphocytes, particularly B lymphocytes). In specific embodiments, Neutrokine-alpha conjugates and/or compositions of the invention have use in the treatment of autoimmune diseases, and in particular, of autoimmune diseases that involve autoantibodies.

The compositions of the invention generally comprise a Neutrokine-alpha protein, associated with either a chelator or a chelator and a metal ion. Herein a Neutrokine-alpha protein associated or linked, directly or indirectly, with a chelator is referred to as “Neutrokine-alpha conjugate.” A “Neutrokine-alpha complex“ herein refers to the association of a metal ion with a Neutrokine-alpha conjugate, preferably via coordination of the metal ion by the chelator. The following sections will describe the Neutrokine-alpha protein, the chelator and metal ion components of the Neutrokine-alpha conjugate and/or Neutrokine-alpha complex.

Neutrokine-alpha Conjugate

The present invention encompasses a Neutrokine-alpha conjugate, wherein said conjugate comprises a Neutrokine-alpha protein and a chelator. Such a conjugate has a number of uses, including diagnostic uses and therapeutic uses, described herein. The Neutrokine-alpha conjugate is also useful for preparing a Neutrokine-alpha complex as disclosed herein. The Neutrokine-alpha conjugate is a Neutrokine-alpha protein covalently bonded to a chelator. The following two sections describe in detail the nature of the Neutrokine-alpha protein, the chelator, and the association of the chelator with the Neutrokine-alpha protein.

The Neutrokine-alpha conjugate can be depicted as NA-(Chel)ₙ, wherein NA is the Neutrokine-alpha protein, Chel is the chelator, and n is the number of chelator molecules attached to said Neutrokine-alpha protein. In specific embodiments, each chelator molecule is directly attached to the Neutrokine-alpha protein. Therefore, if n is 3, the Neutrokine-alpha conjugate contains one Neutrokine-alpha protein with three chelator molecules, wherein each chelator molecule is bonded directly to said Neutrokine-alpha protein.

Neutrokine-alpha Protein

The following section describes the Neutrokine-alpha protein component of the Neutrokine-alpha conjugates and Neutrokine-alpha complexes of the present invention. Nucleotides 147-1001 of SEQ ID NO:1 encode the protein shown in shown in Table 1 (SEQ ID NO:2). The nucleotide sequence of SEQ ID NO:1 was obtained by sequencing the HNEDU15 clone, which was deposited on Oct. 22, 1996 at the American Type Culture Collection, 10801 University Boulevard, Manassas, Va. 20110-2209, and assigned ATCC Accession No. 97768. The deposited clone is contained in the pBluescript SK(−) plasmid (Stratagene, La Jolla, Calif.). The ATCC deposit was made pursuant to the terms of the Budapest Treaty on the International Recognition of the Deposit of Microorganisms for the Purposes of Patent Procedure.

Neutrokine-alpha is a membrane protein which has an intracellular domain corresponding to amino acid 1-46 of SEQ ID NO:2, transmembrane domain (amino acids 47-72 of SEQ ID NO:2), and an extracellular domain (amino acid residues 73-285 of SEQ ID NO:2). Proteolytic cleavage of Neutrokine-alpha from the surface of the cell results in a mature, soluble form of the protein comprising amino acid residues 134-285 of SEQ ID NO:2.

In one embodiment, the Neutrokine-alpha protein is a polypeptide comprising or alternatively, consisting of, an amino acid sequence contained in SEQ ID NO:2, encoded by the cDNA contained in the ATCC Deposit No. 97768, or encoded by nucleic acids which hybridize (e.g., under stringent hybridization conditions) to the nucleotide sequence contained in the ATCC Deposit No. 97768 or the complementary strand thereto. A protein fragment may be “free-standing” or within a larger polypeptide of which the fragment forms a part or region, most preferably as a single continuous region.

In preferred embodiments, the Neutrokine-alpha protein component of the Neutrokine-alpha conjugate or Neutrokine-alpha complex of the present invention corresponds to the extracellular domain of the polypeptide of SEQ ID NO:2 or a fragment or variant thereof. In other preferred embodiments, the Neutrokine-alpha protein component of the Neutrokine-alpha conjugate or Neutrokine-alpha complex of the present invention corresponds to the extracellular domain of the polypeptide encoded by the cDNA contained in ATCC Deposit No. 97768), or a fragment or variant thereof (e.g., the mature, soluble form of the polypeptide encoded by the cDNA contained in ATCC Deposit No 97768. Other forms of Neutrokine-alpha that can act as the Neutrokine-alpha protein component of the Neutrokine-alpha conjugate or Neutrokine-alpha complex of the present invention include the complete polypeptide encoded by the cDNA contained in ATCC Deposit No. 97768 including the intracellular, transmembrane and extracellular domains of the polypeptide encoded by the deposited cDNA, the mature, soluble polypeptide encoded by the deposited cDNA, the extracellular domain minus the intracellular and transmembrane domains of the protein, the complete polypeptide of SEQ ID NO:2, the mature, soluble form of the Neutrokine- alpha protein (e.g., amino acids 134-285 of SEQ ID NO:2 or SEQ ID NO:3), the extracellular domain of Neutrokine-alpha protein (amino acid residues 73-285 of SEQ ID NO:2) minus the intracellular and transmembrane domains, as well as polypeptides which have at least 80%, 85%, 90% similarity, more preferably at least 95% similarity, and still more preferably at least 96%, 97%, 98% or 99% similarity to those described above.

In another embodiment, a Neutrokine-alpha conjugate comprises a Neutrokine-alpha protein and a chelator, wherein said protein includes a polypeptide at least 80%, or at least 85% identical, more preferably at least 90% or 95% identical, still more preferably at least 96%, 97%, 98% or 99% identical to the polypeptide encoded by the deposited cDNA (ATCC Deposit No. 97768) or to the polypeptide of Tables 1 and 2 (SEQ ID NO:2 and SEQ ID NO:3, respectively), and also include portions of such polypeptides that are at least 30 amino acids and more preferably, at least 50 amino acids, in length.

By “% similarity” for two polypeptides, is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a protein having an amino acid sequence at least, for example, 95% “identical” to a reference amino acid sequence of a Neutrokine-alpha protein is intended that the amino acid sequence of the protein is identical to the reference sequence except that the protein sequence may include up to five amino acid alterations per each 100 amino acids of the reference amino acid of the Neutrokine-alpha protein. In other words, to obtain a protein having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide or protein is at least 80%, 85%, 90%, 95%, 96%, 97%, 98% or 99% identical to, for instance, the amino acid sequences shown in Tables 1 and 2, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. Comp. App. Biosci. 6:237-245 (1990). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

Neutrokine alpha, like other members of the TNF family of ligands, forms biologically active trimers. Thus, in highly preferred embodiments, the Neutrokine-alpha protein component of a Neutrokine-alpha conjugate or Neutrokine-alpha complex is a trimer. Neutrokine-alpha may also form higher order structures (Liu et al., Cell 108:383-394 (2002)). Thus, in some embodiments, a Neutrokine-alpha protein may comprise 3, 6, 9, 12, 15, 18, or 60 Neutrokine-alpha monomeric subunits. In another embodiment, the Neutrokine-alpha protein is a mature, soluble Neutrokine-alpha protein. The term “mature, soluble Neutrokine-alpha protein” refers to the portion of the extracellular domain of a Neutrokine-alpha protein that is cleaved from the membrane bound protein. In another embodiment, the Neutrokine-alpha protein is a human mature, soluble Neutrokine-alpha protein (e.g., the protein of SEQ ID NO:3 or a Neutrokine-alpha protein consisting of a trimer of Neutrokine-alpha monomeric subunits wherein each subunit consists of amino acids 134-285 of the protein shown in Table 1 (SEQ ID NO:2)).

TABLE 1
Amino Acid Sequence of Full Length Human Neutrokine-alpha

| | | | | | | |
|---|---|---|---|---|---|---|
| 1 | MDDSTEREQS | RLTSCLKKRE | EMKLKECVSI | LPRKESPSVR | SSKDGKLLAA | TLLLALLSCC |
| 61 | LTVVSFYQVA | ALQGDLASLR | AELQGHHAEK | LPAGAGAPKA | GLEEAPAVTA | GLKIFEPPAP |
| 121 | GEGNSSQNSR | NKRAVQGPEE | TVTQDCLQLI | ADSETPTIQK | GSYTFVPWLL | SFKRGSALEE |
| 181 | KENKILVKET | GYFFIYGQVL | YTDKTYAMGH | LIQRKKVHVF | GDELSLVTLF | RCIQNMPETL |
| 241 | PNNSCYSAGI | AKLEEGDELQ | LAIPRENAQI | SLDGDVTFFG | ALKLL | |

TABLE 2
Amino Acid Sequence of Mature Soluble Human Neutrokine-alpha

| | | | | | | |
|---|---|---|---|---|---|---|
| 1 | | AVQGPEE | TVTQDCLQLI | ADSETPTIQK | GSYTFVPWLL | SFKRGSALEE |
| 61 | KENKILVKET | GYFFIYGQVL | YTDKTYAMGH | LIQRKKVHVF | GDELSLVTLF | RCIQNMPETL |
| 121 | PNNSCYSAGI | AKLEEGDELQ | LAIPRENAQI | SLDGDVTFFG | ALKLL | |

In the most preferred embodiments, the Neutrokine-alpha protein consists of a trimer of identical Neutrokine-alpha monomers each of which has an amino acid sequence that consists of the amino acid sequence shown in Table 2 (SEQ ID NO:3) which corresponds to amino acid residues 134-285 of the human Neutrokine-alpha protein shown in Table 1 (SEQ ID NO:2).

In other preferred embodiments, one or more of the individual monomeric units of a Neutrokine-alpha protein, preferably a Neutrokine-alpha trimer, may consist of a fragment or variant of the protein of SEQ ID NO:3. For example, a monomeric unit of the Neutrokine-alpha protein may consist of a protein that is at least 70% identical to the protein shown in SEQ ID NO:3. In other embodiments, a monomeric unit of the Neutrokine-alpha protein may consist of a protein that is at least 80% identical to the protein shown in SEQ ID NO:3. In preferred embodiments, a monomeric unit of the Neutrokine-alpha protein may consist of a protein that is at least 90% identical to the protein shown in SEQ ID NO:3. And in other preferred embodiments, a monomeric unit of the Neutrokine-alpha protein may consist of a protein that is at least 95% identical to the protein shown in SEQ ID NO:3. In other preferred embodiments, one or more of the individual monomeric units of a Neutrokine-alpha protein, preferably a Neutrokine-alpha trimer, may consist of Neutrokine-alpha protein from another species, e.g., murine, canine, feline, or monkey Neutrokine-alpha. To be useful in the present invention, Neutrokine-alpha proteins must bind to one or more Neutrokine-alpha receptors, such as BAFF-R (SEQ ID NO:5), TACI (SEQ ID NO:7), and/or BCMA (SEQ ID NO:9). Neutrokine-alpha multimers comprising one or more monomers that consist of a fragment or variant of the protein of SEQ ID NO:3 may be tested for their ability to bind a Neutrokine-alpha receptor using any method known in the art including the method shown in Examples 11 and 12.

In other preferred embodiments, one or more of the individual monomeric units of a Neutrokine-alpha protein, preferably a Neutrokine-alpha trimer, comprise a fragment or variant of the protein of SEQ ID NO:3. For example, a monomeric unit of the Neutrokine-alpha protein may comprise a protein that is at least 70% identical to the protein shown in SEQ ID NO:3. In other embodiments, a monomeric unit of the Neutrokine-alpha protein may comprise a protein that is at least 80% identical to the protein shown in SEQ ID NO:3. In preferred embodiments, a monomeric unit of the Neutrokine-alpha protein may comprise a protein that is at least 90% identical to the protein shown in SEQ ID NO:3. And in other preferred embodiments, a monomeric unit of the Neutrokine-alpha protein may comprise a protein that is at least 95% identical to the protein shown in SEQ ID NO:3. In other preferred embodiments, one or more of the individual monomeric units of a Neutrokine-alpha protein, preferably a Neutrokine-alpha trimer, may comprise a Neutrokine-alpha protein from another species, e.g., murine, canine, feline, or monkey Neutrokine-alpha. To be useful in the present invention, Neutrokine-alpha proteins must bind to one or more Neutrokine-alpha receptors, such as BAFF-R (SEQ ID NO:5), TACI (SEQ ID NO:7), and/or BCMA (SEQ ID NO:9). Neutrokine-alpha proteins comprising one or more monomers that comprise a fragment or variant of the protein of SEQ ID NO:3 may be tested for their ability to bind a Neutrokine-alpha receptor using any method known in the art including the method shown in Examples 11 and 12.

In addition to forming homotrimers, Neutrokine-alpha proteins also form heterotrimers with APRIL, another TNF family ligand which is described, for example, in PCT International Publication Number W097/33902 and in Hahne et al., J. Exp. Med. 188:1185-1190 (1998), each of which is herein incorporated by reference in its entirety. The nucleotide and amino acid sequence of APRIL is given in GenBank Accession No. AF046888 (nucleotide) and AAC6132 (protein) and SEQ ID NOS: 10 and 11, respectively. Like Neutrokine-alpha, APRIL is processed into a mature, soluble protein which comprises 3 monomers each consisting of amino acids 105-250 of SEQ ID NO:11. Neutrokine-alpha and APRIL proteins can form heterotrimers wherein each heterotrimer comprises two Neutrokine-alpha monomers and one APRIL monomer. Alternatively, Neutrokine-alpha and APRIL proteins can form heterotrimers wherein each heterotrimer comprises one Neutrokine-alpha monomer and two APRIL monomers. It is preferred that the Neutrokine-alpha monomer(s) in a Neutrokine-alpha/APRIL heterotrimer either comprise, or alternatively consist of, amino acid residues 134-285 of SEQ ID NO:2 or a fragment or variant thereof. It is preferred that the APRIL monomer(s) in a Neutrokine-alpha/APRIL heterotrimer either comprise, or alternatively consist of, amino acid residues 105-250 of SEQ ID NO:11 or a fragment or variant thereof. It is specifically contemplated that a Neutrokine-alpha/APRIL heterotrimers may used as the Neutrokine-alpha protein component of the Neutrokine-alpha conjugates and Neutrokine-alpha complexes of the invention.

In other preferred embodiments, the Neutrokine-alpha protein comprises the amino acid residues shown in Table 1 (SEQ ID NO:2) or a fragment of variant of the protein shown in SEQ ID NO:2 Numerous functional variations of Neutrokine-alpha protein monomers and/or trimers can routinely be made by one of skill in the art. In the present application, a “functional” Neutrokine-alpha protein would be one that was capable of binding to one or more Neutrokine-alpha receptors. International Patent Application Publications WO98/17957, WO00/50597, and WO02/18620 each of which are hereby incorporated by reference in their entireties, describe numerous modifications that can be made to Neutrokine-alpha proteins. For example WO98/17957, WO00/50597, and WO02/18620 describe amino acid substitution, deletion, and addition mutations that can be made to Neutrokine-alpha protein as well as post translational modifications that may be made to Neutrokine-alpha either as a result of natural post-translational mechanisms or as a result of in vitro manipulation of the Neutrokine-alpha protein.

In additional embodiments, the Neutrokine-alpha protein component of a Neutrokine-alpha conjugate or Neutrokine-alpha complex comprises the predicted TNF-conserved domain of Neutrokine-alpha (amino acids 191 to 284 of SEQ ID NO:2).

To improve or alter the characteristics of Neutrokine-alpha proteins which are used in the Neutrokine-alpha conjugates or Neutrokine-alpha complexes of the present invention, protein engineering may be employed. Recombinant DNA technology can be used to create novel mutant proteins, or “muteins,” including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions. For instance, for many proteins, including the extracellular domain or the mature form(s) of a secreted protein, it is known in the art that one or more amino acids may be deleted from the N-terminus or C-terminus without substantial loss of biological function. For instance, Ron et al., J. Biol. Chem., 268:2984-2988 (1993) reported modified KGF proteins that had heparin binding activity even if 3, 8, or 27 amino-terminal amino acid residues were missing.

In the present case, Neutrokine-alpha is a member of the TNF polypeptide family, with a conserved TNF family ligand domain spanning amino acid residues 191-284 of SEQ ID NO:2, deletions of N-terminal amino acids up to the Gly (G) residue at position 191 (in Table 1 (SEQ ID NO:2)) may retain some biological activity such as, for example, the ability to bind to a Neutrokine-alpha receptor or to stimulate lymphocyte (e.g., B cell) proliferation, differentiation, activation, and/or survival to appropriate target cells. Polypeptides having further N-terminal deletions including the Gly (G) residue at position 191 would not be expected to retain biological activities because it is known that this residue in TNF-related polypeptides is in the beginning of the conserved domain required for biological activities. However, even if deletion of one or more amino acids from the N-terminus of a protein results in modification or loss of one or more biological functions of the protein, other functional activities may still be retained. Thus, the ability of the shortened protein to induce and/or bind to antibodies which recognize the complete protein or at least the extracellular domain of the protein generally will be retained when less than the majority of the residues of the complete or extracellular domain of the protein are removed from the N-terminus. Whether a particular polypeptide lacking N-terminal residues of a complete protein retains such immunologic activities can readily be determined by routine methods described herein and/or otherwise known in the art.

Accordingly, the present invention further provides Neutrokine-alpha conjugates and/or Neutrokine-alpha complexes wherein the Neutrokine-alpha protein component has, for example, one or more residues deleted from the amino terminus of the amino acid sequence of the Neutrokine-alpha shown in Table 1 (SEQ ID NO:3), up to the glycine residue at position 191 (Gly-191 residue from the amino terminus). In particular, the present invention further provides Neutrokine-alpha conjugates and/or Neutrokine-alpha complexes wherein the Neutrokine-alpha protein component comprises, or alternatively consists of, the amino acid sequence of residues n¹-285 of SEQ ID NO:2, wherein n¹ is an integer in the range of the amino acid position of amino acid residues 2-190 of the amino acid sequence in SEQ ID NO:2. In specific embodiments, the Neutrokine-alpha conjugates and/or Neutrokine-alpha complexes of the present invention may comprise proteins comprising, or alternatively consisting of, an amino acid sequence selected from the group consisting of residues 2-285, 3-285, 4-285, 5-285, 6-285, 7-285, 8-285, 9-285, 10-285, 11-285, 12-285, 13-285, 14-285, 15-285, 16-285, 17-285, 18-285, 19-285, 20-285, 21-285, 22-285, 23-285, 24-285, 25-285, 26-285, 27-285, 28-285, 29-285, 30-285, 31-285, 32-285, 33-285, 34-285, 35-285, 36-285, 37-285, 38-285, 39-285, 40-285, 41-285, 42-285, 43-285, 44-285, 45-285, 46-285, 47-285, 48-285, 49-285, 50-285, 51-285, 52-285, 53-285, 54-285, 55-285, 56-285, 57-285, 58-285, 59-285, 60-285, 61-285, 62-285, 63-285, 64-285, 65-285, 66-285, 67-285, 68-285, 69-285, 70-285, 71-285, 72-285, 73-285, 74-285, 75-285, 76-285, 77-285, 78-285, 79-285, 80-285, 81-285, 82-285, 83-285, 84-285, 85-285, 86-285, 87-285, 88-285, 89-285, 90-285, 91-285, 92-285, 93-285, 94-285, 95-285, 96-285, 97-285, 98-285, 99-285, 100-285, 101-285, 102-285, 103-285, 104-285, 105-285, 106-285, 107-285, 108-285, 109-285, 110-285, 111-285, 112-285, 113-285, 114-285, 115-285, 116-285, 117-285, 118-285, 119-285, 120-285, 121-285, 122-285, 123-285, 124-285, 125-285, 126-285, 127-285, 128-285, 129-285, 130-285, 131-285, 132-285, 133-285, 134-285, 135-285, 136-285, 137-285, 138-285, 139-285, 140-285, 141-285, 142-285, 143-285, 144-285, 145-285, 146-285, 147-285, 148-285, 149-285, 150-285, 151-285, 152-285, 153-285, 154-285, 155-285, 156-285, 157-285, 158-285, 159-285, 160-285, 161-285, 162-285, 163-285, 164-285, 165-285, 166-285, 167-285, 168-285, 169-285, 170-285, 171-285, 172-285, 173-285, 174-285, 175-285, 176-285, 177-285, 178-285, 179-285, 180-285, 181-285, 182-285, 183-285, 184-285, 185-285, 186-285, 187-285, 188-285, 189-285, and 190-285 of SEQ ID NO:2. The present invention further provides Neutrokine-alpha conjugates and/or Neutrokine-alpha complexes wherein the Neutrokine-alpha protein component comprises, or alternatively, consists of, an amino acid sequence at least 80%, 85%, 90%, 92%, 95%, 96%, 97%, 98% or 99% identical to a Neutrokine-alpha protein described above.

In specific embodiments, the present invention further provides Neutrokine-alpha conjugates and/or Neutrokine-alpha complexes wherein the Neutrokine-alpha protein component comprises, or alternatively consists of, one of the following N-terminally deleted polypeptide fragments of Neutrokine-alpha: amino acid residues Ala-71 through Leu-285, amino acid residues Ala-81 through Leu-285, amino acid residues Leu-112 through Leu-285, amino acid residues Ala-134 through Leu-285, amino acid residues Leu-147 through Leu-285, and amino acid residues Gly-161 through Leu-285 of SEQ ID NO:2.

Similarly, many examples of biologically functional C-terminal deletion muteins are known. For instance, Interferon gamma shows up to ten times higher activities by deleting 8-10 amino acid residues from the carboxy terminus of the protein (Döbeli et al., J. Biotechnology 7:199-216 (1988). Since Neutrokine-alpha protein is a member of the TNF polypeptide family, deletions of C-terminal amino acids up to the leucine residue at position 284 are expected to retain most if not all biological activity such as, for example, ligand binding, the ability to stimulate lymphocyte (e.g., B cell) proliferation, differentiation, and/or activation, and modulation of cell replication. Polypeptides having deletions of up to about 10 additional C-terminal residues (i.e., up to the glycine residue at position 274) also may retain some activity such as receptor binding, although such polypeptides would lack a portion of the conserved TNF domain which extends to about Leu-284 of SEQ ID NO:2. However, even if deletion of one or more amino acids from the C-terminus of a protein results in modification or loss of one or more biological functions of the protein, other functional activities may still be retained. Thus, the ability of the shortened protein to induce and/or bind to antibodies which recognize the complete or mature protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Accordingly, the present invention further provides Neutrokine-alpha conjugates and/or Neutrokine-alpha complexes wherein the Neutrokine-alpha protein component has one or more residues deleted from the carboxy terminus of the amino acid sequence of the Neutrokine-alpha protein shown in Tables 1 (SEQ ID NO:2), up to the glycine residue at position 274 (Gly-274). In particular, the present invention provides Neutrokine-alpha conjugates and/or Neutrokine-alpha complexes wherein the Neutrokine-alpha protein component comprises, or alternatively consists of, the amino acid sequence of residues 1-m¹ of the amino acid sequence in SEQ ID NO:2, where m¹ is any integer in the range of the amino acid position of amino acid residues 274-284 in SEQ ID NO:2. More in particular, the invention provides a conjugate as described above wherein said Neutrokine-alpha protein comprises, or alternatively consists of, an amino acid sequence selected from the group consisting of residues 1-274, 1-275, 1-276, 1-277, 1-278, 1-279, 1-280, 1-281, 1-282, 1-283 and 1-284 of SEQ ID NO:2. The present invention is also directed to Neutrokine-alpha conjugates and/or Neutrokine-alpha complexes wherein the Neutrokine-alpha protein component comprises, or alternatively, consists of, an amino acid sequence at least 80%, 85%, 90%, 92%, 95%, 96%, 97%, 98% or 99% identical to the polypeptide sequence encoding the Neutrokine-alpha proteins described above.

The present invention further provides Neutrokine-alpha conjugates and/or Neutrokine-alpha complexes wherein the Neutrokine-alpha protein component has one or more residues deleted from both the amino and the carboxyl termini, which may be described generally as having residues n¹-m¹ of SEQ ID NO:2, where n¹ and m¹ are integers as defined above.

In additional embodiments, the invention further provides Neutrokine-alpha conjugates and/or Neutrokine-alpha complexes wherein the Neutrokine-alpha protein component comprises, or alternatively consists of amino acid sequence of residues 134-m² of SEQ ID NO:2, where m² is an integer from 140 to 285, corresponding to the position of the amino acid residue in SEQ ID NO:2. For example, the invention provides further provides a conjugate as described above wherein said Neutrokine-alpha protein comprises, or alternatively consists of, an amino acid sequence selected from the group consisting of residues A-134 to Leu-285; A-134 to L-284; A-134 to K-283; A-134 to L-282; A-134 to A-281; A-134 to G-280; A-134 to F-279; A-134 to F-278; A-134 to T-277; A-134 to V-276; A-134 to D-275; A-134 to G-274; A-134 to D-273; A-134 to L-272; A-134 to S-271; A-134 to 1-270; A-134 to Q-269; A-134 to A-268; A-134 to N-267; A-134 to E-266; A-134 to R-265; A-134 to P-264; A-134 to 1-263; A-134 to A-262; A-134 to L-261; A-134 to Q-260; A-134 to L-259; A-134 to E-258; A-134 to D-257; A-134 to G-256; A-134 to E-255; A-134 to E-254; A-134 to L-253; A-134 to K-252; A-134 to A-251; A-134 to I-250; A-134 to G-249; A-134 to A-248; A-134 to S-247; A-134 to Y-246; A-134 to C-245; A-134 to S-244; A-134 to N-243; A-134 to N-242; A-134 to P-241; A-134 to L-240; A-134 to T-239; A-134 to E-238; A-134 to P-237; A-134 to M-236; A-134 to N-235; A-134 to Q-234; A-134 to I-233; A-134 to C-232; A-134 to R-231; A-134 to F-230; A-134 to L-229; A-134 to T-228; A-134 to V-227; A-134 to L-226; A-134 to S-225; A-134 to L-224; A-134 to E-223; A-134 to D-222; A-134 to G-221; A-134 to F-220; A-134 to V-219; A-134 to H-218; A-134 to V-217; A-134 to K-216; A-134 to K-215; A-134 to R-214; A-134 to Q-213; A-134 to 1-212; A-134 to L-211; A-134 to H-210; A-134 to G-209; A-134 to M-208; A-134 to A-207; A-134 to Y-206; A-134 to T-205; A-134 to K-204; A-1 34 to D-203; A-1 34 to T-202; A-134 to Y-201; A-134 to L-200; A-134 to V-199; A-134 to Q-198; A-134 to G-197; A-134 to Y-196; A-134 to I-195; A-134 to F-194; A-134 to F-193; A-134 to Y-192; A-134 to G-191; A-134 to T-190; A-134 to E-189; A-134 to K-188; A-134 to V-187; A-134 to L-186; A-134 to 1-185; A-134 to K-184; A-134 to N-183; A-134 to E-182; A-134 to K-181; A-134 to E-180; A-134 to E-179; A-134 to L-178; A-134 to A-177; A-134 to S-176; A-134 to G-175; A-134 to R-174; A-134 to K-173; A-134 to F-172; A-134 to S-171; A-134 to L-170; A-134 to L-169; A-134 to W-168; A-134 to P-167; A-134 to V-166; A-134 to F-165; A-134 to T-164; A-134 to Y-163; A-134 to S-162; A-134 to G-161; A-134 to K-160; A-134 to Q-159; A-134 to I-158; A-134 to T-157; A-134 to P-156; A-134 to T-155; A-134 to E-154; A-134 to S-153; A-134 to D-152; A-134 to A-151; A-134 to 1-150; A-134 to L-149; A-134 to Q-148; A-134 to L-147; A-134 to C-146; A-134 to D-145; A-134 to Q-144; A-134 to T-143; A-134 to V-142; A-134 to T-141; and A-134 to E-140 of SEQ ID NO:2. The present invention further provides Neutrokine-alpha conjugates and/or Neutrokine-alpha complexes wherein the Neutrokine-alpha protein component comprises, or alternatively, consists of, an amino acid sequence at least 80%, 85%, 90%, 92%, 95%, 96%, 97%, 98% or 99% identical to a Neutrokine-alpha protein described above.

A variant is a protein that may include one or more amino acid substitutions, deletions, or additions, when compared to a reference protein, e.g., SEQ ID NO:3 or SEQ ID NO:2. The substitutions, deletions, or additions may be the result of natural mutations, or human manipulation. Thus, a Neutrokine-alpha protein variant may include one or more amino acid substitutions, deletions or additions, either from natural mutations or human manipulation. As indicated, any change is preferably of a minor nature, such as conservative amino acid substitutions (see Table 3) that do not significantly affect the folding or activity of the protein, in this case the ability of the Neutrokine-alpha protein to bind a Neutrokine-alpha receptor.

TABLE 3
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

For example, site directed changes at the amino acid level of a Neutrokine-alpha protein can be made by replacing a particular amino acid with a conservative substitution. Preferred conservative substitution mutations of the Neutrokine-alpha amino acid sequence provided in Table 3 include: M1 replaced with A, G, I, L, S, T, or V; D2 replaced with E; D3 replaced with E; S4 replaced with A, G, I, L, T, M, or V; T5 replaced with A, G, I, L, S, M, or V; E6 replaced with D; R7 replaced with H, or K; E8 replaced with D; Q9 replaced with N; S10 replaced with A, G, I, L, T, M, or V; R11 replaced with H, or K; L12 replaced with A, G, I, S, T, M, or V; T13 replaced with A, G, I, L, S, M, or V; S14 replaced with A, G, I, L, T, M, or V; L16 replaced with A, G, I, S, T, M, or V; K17 replaced with H, or R; K18 replaced with H, or R; R19 replaced with H, or K; E20 replaced with D; E21 replaced with D; M22 replaced with A, G, I, L, S, T, or V; K23 replaced with H, or R; L24 replaced with A, G, I, S, T, M, or V; K25 replaced with H, or R; E26 replaced with D; V28 replaced with A, G, I, L, S, T, or M; S29 replaced with A, G, I, L, T, M, or V; I30 replaced with A, G, L, S, T, M, or V; L31 replaced with A, G, I, S, T, M, or V; R33 replaced with H, or K; K34 replaced with H, or R; E35 replaced with D; S36 replaced with A, G, I, L, T, M, or V; S38 replaced with A, G, I, L, T, M, or V; V39 replaced with A, G, I, L, S, T, or M; R40 replaced with H, or K; S41 replaced with A, G, I, L, T, M, or V; S42 replaced with A, G, I, L, T, M, or V; K43 replaced with H, or R; D44 replaced with E; G45 replaced with A, I, L, S, T, M, or V; K46 replaced with H, or R; L7 replaced with A, G, I, S, T, M, or V; L48 replaced with A, G, I, S, T, M, or V; A49 replaced with G, I, L, S, T, M, or V; A50 replaced with G, I, L, S, T, M, or V; T51 replaced with A, G, I, L, S, M, or V; L52 replaced with A, G, I, S, T, M, or V; L53 replaced with A, G, I, S, T, M, or V; L54 replaced with A, G, I, S, T, M, or V; A55 replaced with G, I, L, S, T, M, or V; L56 replaced with A, G, I, S, T, M, or V; L57 replaced with A, G, I, S, T, M, or V; S58 replaced with A, G, I, L, T, M, or V; L61 replaced with A, G, I, S, T, M, or V; T62 replaced with A, G, I, L, S, M, or V; V63 replaced with A, G, I, L, S, T, or M; V64 replaced with A, G, I, L, S, T, or M; S65 replaced with A, G, I, L, T, M, or V; F66 replaced with W, or Y; Y67 replaced with F, or W; Q68 replaced with N; V69 replaced with A, G, I, L, S, T, or M; A70 replaced with G, I, L, S, T, M, or V; A71 replaced with G, I, L, S, T, M, or V; L72 replaced with A, G, I, S, T, M, or V; Q73 replaced with N; G74 replaced with A, I, L, S, T, M, or V; D75 replaced with E; L76 replaced with A, G, I, S, T, M, or V; A77 replaced with G, I, L, S, T, M, or V; S78 replaced with A, G, I, L, T, M, or V; L79 replaced with A, G, I, S, T, M, or V; R80 replaced with H, or K; A81 replaced with G, I, L, S, T, M, or V; E82 replaced with D; L83 replaced with A, G, I, S, T, M, or V; Q84 replaced with N; G85 replaced with A, I, L, S, T, M, or V; H86 replaced with K, or R; H87 replaced with K, or R; A88 replaced with G, I, L, S, T, M, or V; E89 replaced with D; K90 replaced with H, or R; L91 replaced with A, G, I, S, T, M, or V; A93 replaced with G, I, L, S, T, M, or V; G94 replaced with A, I, L, S, T, M, or V; A95 replaced with G, I, L, S, T, M, or V; G96 replaced with A, I, L, S, T, M, or V; A97 replaced with G, I, L, S, T, M, or V; K99 replaced with H, or R; A100replaced with G, I, L, S, T, M, or V; G101 replaced with A, I, L, S, T, M, or V; L102 replaced with A, G, I, S, T, M, or V; E103 replaced with D; E104 replaced with D; A105 replaced with G, I, L, S, T, M, or V; A107 replaced with G, I, L, S, T, M, or V; V108 replaced with A, G, I, L, S, T, or M; T109 replaced with A, G, I, L, S, M, or V; A110 replaced with G, I, L, S, T, M, or V; G111 replaced with A, I, L, S, T, M, or V; L112 replaced with A, G, I, S, T, M, or V; K113 replaced with H, or R; I114 replaced with A, G, L, S, T, M, or V; F115 replaced with W, or Y; E116 replaced with D; A119 replaced with G, I, L, S, T, M, or V; G121 replaced with A, I, L, S, T, M, or V; E122 replaced with D; G123 replaced with A, I, L, S, T, M, or V; N124 replaced with Q; S125 replaced with A, G, I, L, T, M, or V; S126 replaced with A, G, I, L, T, M, or V; Q127 replaced with N; N128 replaced with Q; S129 replaced with A, G, I, L, T, M, or V; R130 replaced with H, or K; N131 replaced with Q; K132 replaced with H, or R; R133 replaced with H, or K; A134 replaced with G, I, L, S, T, M, or V; V135 replaced with D, E, H, K, R, N, Q, F, W, Y, P, or C; Q136 replaced with D, E, H, K, R, A, G, I, L, S, T, M, V, F, W, Y, P, or C; G137 replaced with D, E, H, K, R, N, Q, F, W, Y, P, or C; P138 replaced with D, E, H, K, R, A, G, I, L, S, T, M, V, N, Q, F, W, Y, or C; E139 replaced with H, K, R, A, G, I, L, S, T, M, V, N, Q, F, W, Y, P, or C; E140 replaced with H, K, R, A, G, I, L, S, T, M, V, N, Q, F, W, Y, P, or C; T141 replaced with A, G, I, L, S, M, or V; V142 replaced with A, G, I, L, S, T, or M; T143 replaced with A, G, I, L, S, M, or V; Q144 replaced with N; D145 replaced with E; L147 replaced with A, G, I, S, T, M, or V; Q148 replaced with N; L149 replaced with A, G, I, S, T, M, or V; I150 replaced with A, G, L, S, T, M, or V; A151 replaced with G, I, L, S, T, M, or V; D152 replaced with E; S153 replaced with A, G, I, L, T, M, or V; E154 replaced with D; T155 replaced with A, G, I, L, S, M, or V; T157 replaced with A, G, I, L, S, M, or V; I158 replaced with A, G, L, S, T, M, or V; Q159 replaced with N; K160 replaced with H, or R; G161 replaced with A, I, L, S, T, M, or V; S162 replaced with A, G, I, L, T, M, or V; Y163 replaced with F, or W; T164 replaced with A, G, I, L, S, M, or V; F165 replaced with W, or Y; V166 replaced with A, G, I, L, S, T, or M; W168 replaced with F, or Y; L169 replaced with A, G, I, S, T, M, or V; L170 replaced with A, G, I, S, T, M, or V; S171 replaced with A, G, I, L, T, M, or V; F172 replaced with W, or Y; K173 replaced with H, or R; R174 replaced with H, or K; G175 replaced with A, I, L, S, T, M, or V; S176 replaced with A, G, I, L, T, M, or V; A177 replaced with G, I, L, S, T, M, or V; L178 replaced with A, G, I, S, T, M, or V; E179 replaced with D; E180 replaced with D; K181 replaced with H, or R; E182 replaced with D; N183 replaced with Q; K184 replaced with H, or R; I185 replaced with A, G, L, S, T, M, or V; L186 replaced with A, G, I, S, T, M, or V; V187 replaced with A, G, I, L, S, T, or M; K188 replaced with H, or R; E189 replaced with D; T190 replaced with A, G, I, L, S, M, or V; G191 replaced with A, I, L, S, T, M, or V; Y192 replaced with F, or W; F193 replaced with W, or Y; F194 replaced with W, or Y; I195 replaced with A, G, L, S, T, M, or V; Y196 replaced with F, or W; G197 replaced with A, I, L, S, T, M, or V; Q198 replaced with N; V199 replaced with A, G, I, L, S, T, or M; L200 replaced with A, G, I, S, T, M, or V; Y201 replaced with F, or W; T202 replaced with A, G, I, L, S, M, or V; D203 replaced with E; K204 replaced with H, or R; T205 replaced with A, G, I, L, S, M, or V; Y206 replaced with F, or W; A207 replaced with G, I, L, S, T, M, or V; M208 replaced with A, G, I, L, S, T, or V; G209 replaced with A, I, L, S, T, M, or V; H210 replaced with K, or R; L211 replaced with A, G, I, S, T, M, or V; I212 replaced with A, G, L, S, T, M, or V; Q213 replaced with N; R214 replaced with H, or K; K215 replaced with H, or R; K216 replaced with H, or R; V217 replaced with A, G, I, L, S, T, or M; H218 replaced with K, or R; V219 replaced with A, G, I, L, S, T, or M; F220 replaced with W, or Y; G221 replaced with A, I, L, S, T, M, or V; D222 replaced with E; E223 replaced with D; L224 replaced with A, G, I, S, T, M, or V; S225 replaced with A, G, I, L, T, M, or V; L226 replaced with A, G, I, S, T, M, or V; V227 replaced with A, G, I, L, S, T, or M; T228 replaced with A, G, I, L, S, M, or V; L229 replaced with A, G, I, S, T, M, or V; F230 replaced with W, or Y; R231 replaced with H, or K; I233 replaced with A, G, L, S, T, M, or V; Q234 replaced with N; N235 replaced with Q; M236 replaced with A, G, I, L, S, T, or V; E238 replaced with D; T239 replaced with A, G, I, L, S, M, or V; L240 replaced with A, G, I, S, T, M, or V; N242 replaced with Q; N243 replaced with Q; S244 replaced with A, G, I, L, T, M, or V; Y246 replaced with F, or W; S247 replaced with A, G, I, L, T, M, or V; A248 replaced with G, I, L, S, T, M, or V; G249 replaced with A, I, L, S, T, M, or V; I250 replaced with A, G, L, S, T, M, or V; A251 replaced with G, I, L, S, T, M, or V; K252 replaced with H, or R; L253 replaced with A, G, I, S, T, M, or V; E254 replaced with D; E255 replaced with D; G256 replaced with A, I, L, S, T, M, or V; D257 replaced with E; E258 replaced with D; L259 replaced with A, G, I, S, T, M, or V; Q260 replaced with N; L261 replaced with A, G, I, S, T, M, or V; A262 replaced with G, I, L, S, T, M, or V; I263 replaced with A, G, L, S, T, M, or V; R265 replaced with H, or K; E266 replaced with D; N267 replaced with Q; A268 replaced with G, I, L, S, T, M, or V; Q269 replaced with N; I270 replaced with A, G, L, S, T, M, or V; S271 replaced with A, G, I, L, T, M, or V; L272 replaced with A, G, I, S, T, M, or V; D273 replaced with E; G274 replaced with A, I, L, S, T, M, or V; D275 replaced with E; V276 replaced with A, G, I, L, S, T, or M; T277 replaced with A, G, I, L, S, M, or V; F278 replaced with W, or Y; F279 replaced with W, or Y; G280 replaced with A, I, L, S, T, M, or V; A281 replaced with G, I, L, S, T, M, or V; L282 replaced with A, G, I, S, T, M, or V; K283 replaced with H, or R; L284 replaced with A, G, I, S, T, M, or V; and/or L285 replaced with A, G, I, S, T, M, or V. The resulting Neutrokine-alpha proteins may be routinely screened for Neutrokine-alpha functional activity and/or physical properties (such as, for example, the ability to bind one or more Neutrokine-alpha receptors and/or enhanced or reduced stability and/or solubility). The resulting Neutrokine-alpha protein variant may be used in a conjugate as described above.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993)).

A partial, non-limiting, and non-exclusive list of residues of the Neutrokine-alpha protein sequence which may be targeted for mutation includes the following amino acid residues of the Neutrokine-alpha protein sequence as shown in Table 1 (SEQ ID NO:2): V-142; T-143; Q-144; D-145; C-146; L-147; Q-148; L-149; I-150; A-151; D-152; S-153; E-154; T-155; P-156; T-157; 1-158; Q-159; and K-160.

In another embodiment, a Neutrokine-alpha conjugate or Neutrokine-alpha complex of the invention comprises a Neutrokine-alpha fragment or variant, wherein said fragment or variant is a Neutrokine-alpha protein having an amino acid sequence containing one or more non-conservative substitutions of the amino acid sequence provided in SEQ ID NO:2. For example, non-conservative substitutions of the Neutrokine-alpha protein sequence provided in SEQ ID NO:2 include: Ml replaced with D, E, H, K, R, N, Q, F, W, Y, P, or C; D2 replaced with H, K, R, A, G, I, L, S, T, M, V, N, Q, F, W, Y, P, or C; D3 replaced with H, K, R, A, G, I, L, S, T, M, V, N, Q, F, W, Y, P, or C; S4 replaced with D, E, H, K, R, N, Q, F, W, Y, P, or C; T5 replaced with D, E, H, K, R, N, Q, F, W, Y, P, or C; E6 replaced with H, K, R, A, G, I, L, S, T, M, V, N, Q, F, W, Y, P, or C; R7 replaced with D, E, A, G, I, L, S, T, M, V, N, Q, F, W, Y, P, or C; E8 replaced with H, K, R, A, G, I, L, S, T, M, V, N, Q, F, W, Y, P, or C; Q9 replaced with D, E, H, K, R, A, G, I, L, S, T, M, V, F, W, Y, P, or C; S10 replaced with D, E, H, K, R, N, Q, F, W, Y, P, or C; R11 replaced with D, E, A, G, I, L, S, T, M, V, N, Q, F, W, Y, P, or C; L12 replaced with D, E, H, K, R, N, Q, F, W, Y, P, or C; T13 replaced with D, E, H, K, R, N, Q, F, W, Y, P, or C; S14 replaced with D, E, H, K, R, N, Q, F, W, Y, P, or C; C15 replaced with D, E, H, K, R, A, G, I, L, S, T, M, V, N, Q, F, W, Y, or P; L16 replaced with D, E, H, K, R, N, Q, F, W, Y, P, or C; K17 replaced with D, E, A, G, I, L, S, T, M, V, N, Q, F, W, Y, P, or C; K18 replaced with D, E, A, G, I, L, S, T, M, V, N, Q, F, W, Y, P, or C; R19 replaced with D, E, A, G, I, L, S, T, M, V, N, Q, F, W, Y, P, or C; E20 replaced with H, K, R, A, G, I, L, S, T, M, V, N, Q, F, W, Y, P, or C; E21 replaced with H, K, R, A, G, I, L, S, T, M, V, N, Q, F, W, Y, P, or C; M22 replaced with D, E, H, K, R, N, Q, F, W, Y, P, or C; K23 replaced with D, E, A, G, I, L, S, T, M, V, N, Q, F, W, Y, P, or C; L24 replaced with D, E, H, K, R, N, Q, F, W, Y, P, or C; K25 replaced with D, E, A, G, I, L, S, T, M, V, N, Q, F, W, Y, P, or C; E26 replaced with H, K, R, A, G, I, L, S, T, M, V, N, Q, F, W, Y, P, or C; C27 replaced with D, E, H, K, R, A, G, I, L, S, T, M, V, N, Q, F, W, Y, or P; V28 replaced with D, E, H, K, R, N, Q, F, W, Y, P, or C; S29 replaced with D, E, H, K, R, N, Q, F, W, Y, P, or C; I30 replaced with D, E, H, K, R, N, Q, F, W, Y, P, or C; L31 replaced with D, E, H, K, R, N, Q, F, W, Y, P, or C; P32 replaced with D, E, H, K, R, A, G, I, L, S, T, M, V, N, Q, F, W, Y, or C; R33 replaced with D, E, A, G, I, L, S, T, M, V, N, Q, F, W, Y, P, or C; K34 replaced with D, E, A, G, I, L, S, T, M, V, N, Q, F, W, Y, P, or C; E35 replaced with H, K, R, A, G, I, L, S, T, M, V, N, Q, F, W, Y, P, or C; S36 replaced with D, E, H, K, R, N, Q, F, W, Y, P, or C; P37 replaced with D, E, H, K, R, A, G, I, L, S, T, M, V, N, Q, F, W, Y, or C; S38 replaced with D, E, H, K, R, N, Q, F, W, Y, P, or C; V39 replaced with D, E, H, K, R, N, Q, F, W, Y, P, or C; R40 replaced with D, E, A, G, I, L, S, T, M, V, N, Q, F, W, Y, P, or C; S41 replaced with D, E, H, K, R, N, Q, F, W, Y, P, or C; S42 replaced with D, E, H, K, R, N, Q, F, W, Y, P, or C; K43 replaced with D, E, A, G, I, L, S, T, M, V, N, Q, F, W, Y, P, or C; D44 replaced with H, K, R, A, G, I, L, S, T, M, V, N, Q, F, W, Y, P, or C; G45 replaced with D, E, H, K, R, N, Q, F, W, Y, P, or C; K46 replaced with D, E, A, G, I, L, S, T, M, V, N, Q, F, W, Y, P, or C; L47 replaced with D, E, H, K, R, N, Q, F, W, Y, P, or C; L48 replaced with D, E, H, K, R, N, Q, F, W, Y, P, or C; A49 replaced with D, E, H, K, R, N, Q, F, W, Y, P, or C; A50 replaced with D, E, H, K, R, N, Q, F, W, Y, P, or C; T51 replaced with D, E, H, K, R, N, Q, F, W, Y, P, or C; L52 replaced with D, E, H, K, R, N, Q, F, W, Y, P, or C; L53 replaced with D, E, H, K, R, N, Q, F, W, Y, P, or C; L54 replaced with D, E, H, K, R, N, Q, F, W, Y, P, or C; A55 replaced with D, E, H, K, R, N, Q, F, W, Y, P, or C; L56 replaced with D, E, H, K, R, N, Q, F, W, Y, P, or C; L57 replaced with D, E, H, K, R, N, Q, F, W, Y, P, or C; S58 replaced with D, E, H, K, R, N, Q, F, W, Y, P, or C; C59 replaced with D, E, H, K, R, A, G, I, L, S, T, M, V, N, Q, F, W, Y, or P; C60 replaced with D, E, H, K, R, A, G, I, L, S, T, M, V, N, Q, F, W, Y, or P; L61 replaced with D, E, H, K, R, N, Q, F, W, Y, P, or C; T62 replaced with D, E, H, K, R, N, Q, F, W, Y, P, or C; V63 replaced with D, E, H, K, R, N, Q, F, W, Y, P, or C; V64 replaced with D, E, H, K, R, N, Q, F, W, Y, P, or C; S65 replaced with D, E, H, K, R, N, Q, F, W, Y, P, or C; F66 replaced with D, E, H, K, R, N, Q, A, G, I, L, S, T, M, V, P, or C; Y67 replaced with D, E, H, K, R, N, Q, A, G, I, L, S, T, M, V, P, or C; Q68 replaced with D, E, H, K, R, A, G, I, L, S, T, M, V, F, W, Y, P, or C; V69 replaced with D, E, H, K, R, N, Q, F, W, Y, P, or C; A70 replaced with D, E, H, K, R, N, Q, F, W, Y, P, or C; A71 replaced with D, E, H, K, R, N, Q, F, W, Y, P, or C; L72 replaced with D, E, H, K, R, N, Q, F, W, Y, P, or C; Q73 replaced with D, E, H, K, R, A, G, I, L, S, T, M, V, F, W, Y, P, or C; G74 replaced with D, E, H, K, R, N, Q, F, W, Y, P, or C; D75 replaced with H, K, R, A, G, I, L, S, T, M, V, N, Q, F, W, Y, P, or C; L76 replaced with D, E, H, K, R, N, Q, F, W, Y, P, or C; A77 replaced with D, E, H, K, R, N, Q, F, W, Y, P, or C; S78 replaced with D, E, H, K, R, N, Q, F, W, Y, P, or C; L79 replaced with D, E, H, K, R, N, Q, F, W, Y, P, or C; R80 replaced with D, E, A, G, I, L, S, T, M, V, N, Q, F, W, Y, P, or C; A81 replaced with D, E, H, K, R, N, Q, F, W, Y, P, or C; E82 replaced with H, K, R, A, G, I, L, S, T, M, V, N, Q, F, W, Y, P, or C; L83 replaced with D, E, H, K, R, N, Q, F, W, Y, P, or C; Q84 replaced with D, E, H, K, R, A, G, I, L, S, T, M, V, F, W, Y, P, or C; G85 replaced with D, E, H, K, R, N, Q, F, W, Y, P, or C; H86 replaced with D, E, A, G, I, L, S, T, M, V, N, Q, F, W, Y, P, or C; H87 replaced with D, E, A, G, I, L, S, T, M, V, N, Q, F, W, Y, P, or C; A88 replaced with D, E, H, K, R, N, Q, F, W, Y, P, or C; E89 replaced with H, K, R, A, G, I, L, S, T, M, V, N, Q, F, W, Y, P, or C; K90 replaced with D, E, A, G, I, L, S, T, M, V, N, Q, F, W, Y, P, or C; L91 replaced with D, E, H, K, R, N, Q, F, W, Y, P, or C; P92 replaced with D, E, H, K, R, A, G, I, L, S, T, M, V, N, Q, F, W, Y, or C; A93 replaced with D, E, H, K, R, N, Q, F, W, Y, P, or C; G94 replaced with D, E, H, K, R, N, Q, F, W, Y, P, or C; A95 replaced with D, E, H, K, R, N, Q, F, W, Y, P, or C; G96 replaced with D, E, H, K, R, N, Q, F, W, Y, P, or C; A97 replaced with D, E, H, K, R, N, Q, F, W, Y, P, or C; P98 replaced with D, E, H, K, R, A, G, I, L, S, T, M, V, N, Q, F, W, Y, or C; K99 replaced with D, E, A, G, I, L, S, T, M, V, N, Q, F, W, Y, P, or C; A100 replaced with D, E, H, K, R, N, Q, F, W, Y, P, or C; G101 replaced with, D, E, H, K, R, N, Q, F, W, Y, P, or C; L102 replaced with D, E, H, K, R, N, Q, F, W, Y, P, or C; E103 replaced with H, K, R, A, G, I, L, S, T, M, V, N, Q, F, W, Y, P, or C; E104 replaced with H, K, R, A, G, I, L, S, T, M, V, N, Q, F, W, Y, P, or C; A105 replaced with D, E, H, K, R, N, Q, F, W, Y, P, or C; P106 replaced with D, E, H, K, R, A, G, I, L, S, T, M, V, N, Q, F, W, Y, or C; A107 replaced with D, E, H, K, R, N, Q, F, W, Y, P, or C; V108 replaced with D, E, H, K, R, N, Q, F, W, Y, P, or C; T109 replaced with D, E, H, K, R, N, Q, F, W, Y, P, or C; A110 replaced with D, E, H, K, R, N, Q, F, W, Y, P, or C; G111 replaced with D, E, H, K, R, N, Q, F, W, Y, P, or C; L 112 replaced with D, E, H, K, R, N, Q, F, W, Y, P, or C; K 113 replaced with D, E, A, G, I, L, S, T, M, V, N, Q, F, W, Y, P, or C; I114 replaced with D, E, H, K, R, N, Q, F, W, Y, P, or C; F115 replaced with D, E, H, K, R, N, Q, A, G, I, L, S, T, M, V, P, or C; E116 replaced with H, K, R, A, G, I, L, S, T, M, V, N, Q, F, W, Y, P, or C; P117 replaced with D, E, H, K, R, A, G, I, L, S, T, M, V, N, Q, F, W, Y, or C; P118 replaced with D, E, H, K, R, A, G, I, L, S, T, M, V, N, Q, F, W, Y, or C; A119 replaced with D, E, H, K, R, N, Q, F, W, Y, P, or C; P120 replaced with D, E, H, K, R, A, G, I, L, S, T, M, V, N, Q, F, W, Y, or C; G121 replaced with D, E, H, K, R, N, Q, F, W, Y, P, or C; E122 replaced with H, K, R, A, G, I, L, S, T, M, V, N, Q, F, W, Y, P, or C; G123 replaced with D, E, H, K, R, N, Q, F, W, Y, P, or C; N124 replaced with D, E, H, K, R, A, G, I, L, S, T, M, V, F, W, Y, P, or C; S125 replaced with D, E, H, K, R, N, Q, F, W, Y, P, or C; S126 replaced with D, E, H, K, R, N, Q, F, W, Y, P, or C; Q127 replaced with D, E, H, K, R, A, G, I, L, S, T, M, V, F, W, Y, P, or C; N128 replaced with D, E, H, K, R, A, G, I, L, S, T, M, V, F, W, Y, P, or C; S129 replaced with D, E, H, K, R, N, Q, F, W, Y, P, or C; R130 replaced with D, E, A, G, I, L, S, T, M, V, N, Q, F, W, Y, P, or C; N131 replaced with D, E, H, K, R, A, G, I, L, S, T, M, V, F, W, Y, P, or C; K132 replaced with D, E, A, G, I, L, S, T, M, V, N, Q, F, W, Y, P, or C; R133 replaced with D, E, A, G, I, L, S, T, M, V, N, Q, F, W, Y, P, or C; A134 replaced with D, E, H, K, R, N, Q, F, W, Y, P, or C; V135 replaced with D, E, H, K, R, N, Q, F, W, Y, P, or C; Q136 replaced with D, E, H, K, R, A, G, I, L, S, T, M, V, F, W, Y, P, or C; G137 replaced with D, E, H, K, R, N, Q, F, W, Y, P, or C; P138 replaced with D, E, H, K, R, A, G, I, L, S, T, M, V, N, Q, F, W, Y, or C; E139 replaced with H, K, R, A, G, I, L, S, T, M, V, N, Q, F, W, Y, P, or C; E140 replaced with H, K, R, A, G, I, L, S, T, M, V, N, Q, F, W, Y, P, or C; T141 replaced with D, E, H, K, R, N, Q, F, W, Y, P, or C; V142 replaced with D, E, H, K, R, N, Q, F, W, Y, P, or C; T143 replaced with D, E, H, K, R, N, Q, F, W, Y, P, or C; Q144 replaced with D, E, H, K, R, A, G, I, L, S, T, M, V, F, W, Y, P, or C; D145 replaced with H, K, R, A, G, I, L, S, T, M, V, N, Q, F, W, Y, P, or C; C146 replaced with D, E, H, K, R, A, G, I, L, S, T, M, V, N, Q, F, W, Y, or P; L147 replaced with D, E, H, K, R, N, Q, F, W, Y, P, or C; Q148 replaced with D, E, H, K, R, A, G, I, L, S, T, M, V, F, W, Y, P, or C; L149 replaced with D, E, H, K, R, N, Q, F, W, Y, P, or C; I150 replaced with D, E, H, K, R, N, Q, F, W, Y, P, or C; A151 replaced with D, E, H, K, R, N, Q, F, W, Y, P, or C; D152 replaced with H, K, R, A, G, I, L, S, T, M, V, N, Q, F, W, Y, P, or C; S153 replaced with D, E, H, K, R, N, Q, F, W, Y, P, or C; E154 replaced with H, K, R, A, G, I, L, S, T, M, V, N, Q, F, W, Y, P, or C; T155 replaced with D, E, H, K, R, N, Q, F, W, Y, P, or C; P156 replaced with D, E, H, K, R, A, G, I, L, S, T, M, V, N, Q, F, W, Y, or C; T157 replaced with D, E, H, K, R, N, Q, F, W, Y, P, or C; I158 replaced with D, E, H, K, R, N, Q, F, W, Y, P, or C; Q159 replaced with D, E, H, K, R, A, G, I, L, S, T, M, V, F, W, Y, P, or C; K160 replaced with D, E, A, G, I, L, S, T, M, V, N, Q, F, W, Y, P, or C; G161 replaced with D, E, H, K, R, N, Q, F, W, Y, P, or C; S162 replaced with D, E, H, K, R, N, Q, F, W, Y, P, or C; Y163 replaced with D, E, H, K, R, N, Q, A, G, I, L, S, T, M, V, P, or C; T164 replaced with D, E, H, K, R, N, Q, F, W, Y, P, or C; F165 replaced with D, E, H, K, R, N, Q, A, G, I, L, S, T, M, V, P, or C; V166 replaced with D, E, H, K, R, N, Q, F, W, Y, P, or C; P167 replaced with D, E, H, K, R, A, G, I, L, S, T, M, V, N, Q, F, W, Y, or C; W168 replaced with D, E, H, K, R, N, Q, A, G, I, L, S, T, M, V, P, or C; L169 replaced with D, E, H, K, R, N, Q, F, W, Y, P, or C; L170 replaced with D, E, H, K, R, N, Q, F, W, Y, P, or C; S171 replaced with D, E, H, K, R, N, Q, F, W, Y, P, or C; F172 replaced with D, E, H, K, R, N, Q, A, G, I, L, S, T, M, V, P, or C; K173 replaced with D, E, A, G, I, L, S, T, M, V, N, Q, F, W, Y, P, or C; R174 replaced with D, E, A, G, I, L, S, T, M, V, N, Q, F, W, Y, P, or C; G175 replaced with D, E, H, K, R, N, Q, F, W, Y, P, or C; S176 replaced with D, E, H, K, R, N, Q, F, W, Y, P, or C; A177 replaced with D, E, H, K, R, N, Q, F, W, Y, P, or C; L178 replaced with D, E, H, K, R, N, Q, F, W, Y, P, or C; E179 replaced with H, K, R, A, G, I, L, S, T, M, V, N, Q, F, W, Y, P, or C; E180 replaced with H, K, R, A, G, I, L, S, T, M, V, N, Q, F, W, Y, P, or C; K181 replaced with D, E, A, G, I, L, S, T, M, V, N, Q, F, W, Y, P, or C; E182 replaced with H, K, R, A, G, I, L, S, T, M, V, N, Q, F, W, Y, P, or C; N183 replaced with D, E, H, K, R, A, G, I, L, S, T, M, V, F, W, Y, P, or C; K184 replaced with D, E, A, G, I, L, S, T, M, V, N, Q, F, W, Y, P, or C; I185 replaced with D, E, H, K, R, N, Q, F, W, Y, P, or C; L186 replaced with D, E, H, K, R, N, Q, F, W, Y, P, or C; V187 replaced with D, E, H, K, R, N, Q, F, W, Y, P, or C; K188 replaced with D, E, A, G, I, L, S, T, M, V, N, Q, F, W, Y, P, or C; E189 replaced with H, K, R, A, G, I, L, S, T, M, V, N, Q, F, W, Y, P, or C; T190 replaced with D, E, H, K, R, N, Q, F, W, Y, P, or C; G191 replaced with D, E, H, K, R, N, Q, F, W, Y, P, or C; Y192 replaced with D, E, H, K, R, N, Q, A, G, I, L, S, T, M, V, P, or C; F193 replaced with D, E, H, K, R, N, Q, A, G, I, L, S, T, M, V, P, or C; F194 replaced with D, E, H, K, R, N, Q, A, G, I, L, S, T, M, V, P, or C; I195 replaced with D, E, H, K, R, N, Q, F, W, Y, P, or C; Y196 replaced with D, E, H, K, R, N, Q, A, G, I, L, S, T, M, V, P, or C; G197 replaced with D, E, H, K, R, N, Q, F, W, Y, P, or C; Q198 replaced with D, E, H, K, R, A, G, I, L, S, T, M, V, F, W, Y, P, or C; V199 replaced with D, E, H, K, R, N, Q, F, W, Y, P, or C; L200 replaced with D, E, H, K, R, N, Q, F, W, Y, P, or C; Y201 replaced with D, E, H, K, R, N, Q, A, G, I, L, S, T, M, V, P, or C; T202 replaced with D, E, H, K, R, N, Q, F, W, Y, P, or C; D203 replaced with H, K, R, A, G, I, L, S, T, M, V, N, Q, F, W, Y, P, or C; K204 replaced with D, E, A, G, I, L, S, T, M, V, N, Q, F, W, Y, P, or C; T205 replaced with D, E, H, K, R, N, Q, F, W, Y, P, or C; Y206 replaced with D, E, H, K, R, N, Q, A, G, I, L, S, T, M, V, P, or C; A207 replaced with D, E, H, K, R, N, Q, F, W, Y, P, or C; M208 replaced with D, E, H, K, R, N, Q, F, W, Y, P, or C; G209 replaced with D, E, H, K, R, N, Q, F, W, Y, P, or C; H210 replaced with D, E, A, G, I, L, S, T, M, V, N, Q, F, W, Y, P, or C; L211 replaced with D, E, H, K, R, N, Q, F, W, Y, P, or C; I212 replaced with D, E, H, K, R, N, Q, F, W, Y, P, or C; Q213 replaced with D, E, H, K, R, A, G, I, L, S, T, M, V, F, W, Y, P, or C; R214 replaced with D, E, A, G, I, L, S, T, M, V, N, Q, F, W, Y, P, or C; K215 replaced with D, E, A, G, I, L, S, T, M, V, N, Q, F, W, Y, P, or C; K216 replaced with D, E, A, G, I, L, S, T, M, V, N, Q, F, W, Y, P, or C; V217 replaced with D, E, H, K, R, N, Q, F, W, Y, P, or C; H218 replaced with D, E, A, G, I, L, S, T, M, V, N, Q, F, W, Y, P, or C; V219 replaced with D, E, H, K, R, N, Q, F, W, Y, P, or C; F220 replaced with D, E, H, K, R, N, Q, A, G, I, L, S, T, M, V, P, or C; G221 replaced with D, E, H, K, R, N, Q, F, W, Y, P, or C; D222 replaced with H, K, R, A, G, I, L, S, T, M, V, N, Q, F, W, Y, P, or C; E223 replaced with H, K, R, A, G, I, L, S, T, M, V, N, Q, F, W, Y, P, or C; L224 replaced with D, E, H, K, R, N, Q, F, W, Y, P, or C; S225 replaced with D, E, H, K, R, N, Q, F, W, Y, P, or C; L226 replaced with D, E, H, K, R, N, Q, F, W, Y, P, or C; V227 replaced with D, E, H, K, R, N, Q, F, W, Y, P, or C; T228 replaced with D, E, H, K, R, N, Q, F, W, Y, P, or C; L229 replaced with D, E, H, K, R, N, Q, F, W, Y, P, or C; F230 replaced with D, E, H, K, R, N, Q, A, G, I, L, S, T, M, V, P, or C; R231 replaced with D, E, A, G, I, L, S, T, M, V, N, Q, F, W, Y, P, or C; C232 replaced with D, E, H, K, R, A, G, I, L, S, T, M, V, N, Q, F, W, Y, or P; I233 replaced with D, E, H, K, R, N, Q, F, W, Y, P, or C; Q234 replaced with D, E, H, K, R, A, G, I, L, S, T, M, V, F, W, Y, P, or C; N235 replaced with D, E, H, K, R, A, G, I, L, S, T, M, V, F, W, Y, P, or C; M236 replaced with D, E, H, K, R, N, Q, F, W, Y, P, or C; P237 replaced with D, E, H, K, R, A, G, I, L, S, T, M, V, N, Q, F, W, Y, or C; E238 replaced with H, K, R, A, G, I, L, S, T, M, V, N, Q, F, W, Y, P, or C; T239 replaced with D, E, H, K, R, N, Q, F, W, Y, P, or C; L240 replaced with D, E, H, K, R, N, Q, F, W, Y, P, or C; P241 replaced with D, E, H, K, R, A, G, I, L, S, T, M, V, N, Q, F, W, Y, or C; N242 replaced with D, E, H, K, R, A, G, I, L, S, T, M, V, F, W, Y, P, or C; N243 replaced with D, E, H, K, R, A, G, I, L, S, T, M, V, F, W, Y, P, or C; S244 replaced with D, E, H, K, R, N, Q, F, W, Y, P, or C; C245 replaced with D, E, H, K, R, A, G, I, L, S, T, M, V, N, Q, F, W, Y, or P; Y246 replaced with D, E, H, K, R, N, Q, A, G, I, L, S, T, M, V, P, or C; S247 replaced with D, E, H, K, R, N, Q, F, W, Y, P, or C; A248 replaced with D, E, H, K, R, N, Q, F, W, Y, P, or C; G249 replaced with D, E, H, K, R, N, Q, F, W, Y, P, or C; I250 replaced with D, E, H, K, R, N, Q, F, W, Y, P, or C; A251 replaced with D, E, H, K, R, N, Q, F, W, Y, P, or C; K252 replaced with D, E, A, G, I, L, S, T, M, V, N, Q, F, W, Y, P, or C; L253 replaced with D, E, H, K, R, N, Q, F, W, Y, P, or C; E254 replaced with H, K, R, A, G, I, L, S, T, M, V, N, Q, F, W, Y, P, or C; E255 replaced with H, K, R, A, G, I, L, S, T, M, V, N, Q, F, W, Y, P, or C; G256 replaced with D, E, H, K, R, N, Q, F, W, Y, P, or C; D257 replaced with H, K, R, A, G, I, L, S, T, M, V, N, Q, F, W, Y, P, or C; E258 replaced with H, K, R, A, G, I, L, S, T, M, V, N, Q, F, W, Y, P, or C; L259 replaced with D, E, H, K, R, N, Q, F, W, Y, P, or C; Q260 replaced with D, E, H, K, R, A, G, I, L, S, T, M, V, F, W, Y, P, or C; L261 replaced with D, E, H, K, R, N, Q, F, W, Y, P, or C; A262 replaced with D, E, H, K, R, N, Q, F, W, Y, P, or C; I263 replaced with D, E, H, K, R, N, Q, F, W, Y, P, or C; P264 replaced with D, E, H, K, R, A, G, I, L, S, T, M, V, N, Q, F, W, Y, or C; R265 replaced with D, E, A, G, I, L, S, T, M, V, N, Q, F, W, Y, P, or C; E266 replaced with H, K, R, A, G, I, L, S, T, M, V, N, Q, F, W, Y, P, or C; N267 replaced with D, E, H, K, R, A, G, I, L, S, T, M, V, F, W, Y, P, or C; A268 replaced with D, E, H, K, R, N, Q, F, W, Y, P, or C; Q269 replaced with D, E, H, K, R, A, G, I, L, S, T, M, V, F, W, Y, P, or C; I270 replaced with D, E, H, K, R, N, Q, F, W, Y, P, or C; S271 replaced with D, E, H, K, R, N, Q, F, W, Y, P, or C; L272 replaced with D, E, H, K, R, N, Q, F, W, Y, P, or C; D273 replaced with H, K, R, A, G, I, L, S, T, M, V, N, Q, F, W, Y, P, or C; G274 replaced with D, E, H, K, R, N, Q, F, W, Y, P, or C; D275 replaced with H, K, R, A, G, I, L, S, T, M, V, N, Q, F, W, Y, P, or C; V276 replaced with D, E, H, K, R, N, Q, F, W, Y, P, or C; T277 replaced with D, E, H, K, R, N, Q, F, W, Y, P, or C; F278 replaced with D, E, H, K, R, N, Q, A, G, I, L, S, T, M, V, P, or C; F279 replaced with D, E, H, K, R, N, Q, A, G, I, L, S, T, M, V, P, or C; G280 replaced with D, E, H, K, R, N, Q, F, W, Y, P, or C; A281 replaced with D, E, H, K, R, N, Q, F, W, Y, P, or C; L282 replaced with D, E, H, K, R, N, Q, F, W, Y, P, or C; K283 replaced with D, E, A, G, I, L, S, T, M, V, N, Q, F, W, Y, P, or C; L284 replaced with D, E, H, K, R, N, Q, F, W, Y, P, or C; and/or L285 replaced with D, E, H, K, R, N, Q, F, W, Y, P, or C. The resulting Neutrokine-alpha protein, Neutrokine-alpha conjugate, or Neutrokine-alpha complex of the invention may be routinely screened for Neutrokine-alpha functional activities and/or physical properties (such as, for example, the ability to bind one or more Neutrokine-alpha receptors and/or enhanced or reduced stability and/or solubility) described throughout the specification and known in the art. In one embodiment, the resulting Neutrokine-alpha protein, Neutrokine-alpha conjugate, or Neutrokine-alpha complex of the invention has either and increased or decreased Neutrokine-alpha functional activity while maintaining the ability to bind to a Neutrokine-alpha receptor and be internalized into a cell.

The resulting Neutrokine-alpha conjugate may be routinely screened for Neutrokine-alpha functional activities and/or physical properties (such as, for example, enhanced or reduced stability and/or solubility) described throughout the specification and known in the art.

A partial, non-limiting and non-exclusive list of residues of the Neutrokine-alpha protein sequence which may be targeted for mutation includes one or more of the following amino acid residues of the Neutrokine-alpha protein sequence as shown in Table 1 (SEQ ID NO:2): V-142; T-143; Q-144; D-145; C-146; L-147; Q-148; L-149; 1-150; A-151; D-152; S-153; E-154; T-155; P-156; T-157; 1-158; Q-159; and K-160.

Thus, the invention also encompasses Neutrokine-alpha conjugates and Neutrokine-alpha complexes wherein the Neutrokine-alpha protein component has one or more amino acid residues deleted, added, or substituted to generate Neutrokine-alpha protein that is better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognitions sequences in the Neutrokine-alpha protein, and/or an amino acid deletion at the second position of any one or more such recognition sequences will prevent glycosylation of the Neutrokine-alpha at the modified tripeptide sequence (see, e.g., Miyajimo et al., EMBO J 5:1193-1197). By way of a non-limiting example, mutation of the serine at position 244 to alanine either singly or in combination with mutation of the asparagine at position 242 to glutamine abolishes glycosylation of the mature, soluble form of Neutrokine-alpha (amino acids 134-285) of SEQ ID NO:2) when expressed in the yeast Pichea pastoris. A mutant Neutrokine-alpha protein in which only the asparagine at position 242 is mutated to glutamine is still glycosylated when expressed in Pichea pastoris. In this mutant, the glycosylation event may be due to the activation or unmasking of an O-linked glyscosylation site at serine 244.

Additionally, one or more of the amino acid residues of a Neutrokine-alpha protein (e.g., arginine and lysine residues) may be deleted or substituted with another residue to eliminate undesired processing by proteases such as, for example, furins or kexins. One possible result of such a mutation is that Neutrokine-alpha protein is not cleaved and released from the cell surface.

In a specific embodiment, Lys-132 and/or Arg-133 of the Neutrokine-alpha sequence shown in SEQ ID NO:2 is mutated to another amino acid residue, or deleted altogether, to prevent or diminish release of the soluble form of Neutrokine-alpha from cells expressing Neutrokine-alpha. In a more specific embodiment, Lys-132 of the Neutrokine-alpha sequence shown in SEQ ID NO:2 is mutated to Ala-132. In another, nonexclusive specific embodiment, Arg-133 of the Neutrokine-alpha sequence shown in SEQ ID NO:2 is mutated to Ala-133. These mutated proteins have uses such as, for example, in ex vivo therapy or gene therapy, to engineer cells expressing a Neutrokine-alpha polypeptide that is retained on the surface of the engineered cells.

In a specific embodiment, Cys-146 of the Neutrokine-alpha sequence shown in SEQ ID NO: 1 or SEQ ID NO:2 is mutated to another amino acid residue, or deleted altogether, for example, to aid preventing or diminishing oligomerization of the mutant Neutrokine-alpha protein when expressed in an expression system. In a specific embodiment, Cys-146 is replaced with a serine amino acid residue.

In another specific embodiment, Cys-232 of the Neutrokine-alpha sequence shown in SEQ ID NO:1 or SEQ ID NO:2 is mutated to another amino acid residue, or deleted altogether, for example, to aid preventing or diminishing oligomerization of the mutant Neutrokine-alpha protein when expressed in an expression system (essentially as described in Example 1). In a specific embodiment, Cys-232 is replaced with a serine amino acid residue.

In yet another specific embodiment, Cys-245 of the Neutrokine-alpha sequence shown in SEQ ID NO: 1 or SEQ ID NO:2 is mutated to another amino acid residue, or deleted altogether, for example, to aid preventing or diminishing oligomerization of the mutant Neutrokine-alpha protein when expressed in an expression system (essentially as described in Example 1). In a specific embodiment, Cys-245 is replaced with a serine amino acid residue.

As is known in the art, many peptides and proteins may contain carbohydrates attached to the peptide or protein. The Neutrokine-alpha protein used in the present invention may optionally contain one or more carbohydrates attached.

Vectors and Host Cells For Producing Neutrokine-alpha

The present invention also relates to vectors which include the isolated DNA molecules of the present invention, host cells which are genetically engineered with the recombinant vectors, or which are otherwise engineered to produce the polypeptides of the invention, and the production of Neutrokine-alpha and/or Neutrokine-alphaSV polypeptides, or fragments thereof, by recombinant or synthetic techniques.

In one embodiment, the polynucleotides of the invention are joined to a vector (e.g., a cloning or expression vector). The vector may be, for example, a phage, plasmid, viral or retroviral vector. Retroviral vectors may be replication competent or replication defective. In the latter case, viral propagation generally will occur only in complementing host cells. The polynucleotides may be joined to a vector containing a selectable marker for propagation in a host. Introduction of the vector construct into the host cell can be effected by techniques known in the art which include, but are not limited to, calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, for example, stabilization or simplified purification of expressed recombinant product.

In one embodiment, the DNA of the invention is operatively associated with an appropriate heterologous regulatory element (e.g., promoter or enhancer), such as, the phage lambda PL promoter, the E. coli lac, trp, phoA, and tac promoters, the SV40 early and late promoters and promoters of retroviral LTRs, to name a few. Other suitable promoters will be known to the skilled artisan.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

The host cell can be a higher eukaryotic cell, such as a mammalian cell (e.g., a human derived cell), or a lower eukaryotic cell, such as a yeast cell, or the host cell can be a prokaryotic cell, such as a bacterial cell. The host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed. Selection of appropriate vectors and promoters for expression in a host cell is a well-known procedure and the requisite techniques for expression vector construction, introduction of the vector into the host and expression in the host are routine skills in the art.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium, and various species within the genera Pseudomonas, Streptomyces, and Staphylococcus, although others may also be employed as a matter of choice. As a representative, but nonlimiting example, useful expression vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed. Among vectors preferred for use in bacteria include pHE4-5 (ATCC Accession No. 209311; and variations thereof), pQE70, pQE60 and pQE-9, available from QIAGEN, Inc., supra; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Preferred expression vectors for use in yeast systems include, but are not limited to, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, pPIC9K, and PAO815 (all available from Invitrogen, Carlsbad, Calif.). Among preferred eukaryotic vectors are pWLNEO, pSV2CAT, pOG44, pXT1 and pSG available from Stratagene; and pSVK3, pBPV, pMSG and pSVL (available from Pharmacia). Other suitable vectors will be readily apparent to the skilled artisan.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

In one embodiment, the yeast Pichia pastoris is used to express Neutrokine-alpha protein in a eukaryotic system. Pichia pastoris is a methylotrophic yeast which can metabolize methanol as its sole carbon source. A main step in the methanol metabolization pathway is the oxidation of methanol to formaldehyde using O₂. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a Neutrokine-alpha or Neutrokine-alphaSV polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a Neutrokine-alpha or Neutrokine-alphaSV polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a Neutrokine-alpha or Neutrokine-alphaSV protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG as required.

In one embodiment, high-level expression of a heterologous coding sequence, such as, for example, a Neutrokine-alpha or Neutrokine-alphaSV polynucleotide of the present invention, may be achieved by cloning the heterologous polynucleotide of the invention into an expression vector such as, for example, pGAPZ or pGAPZalpha, and growing the yeast culture in the absence of methanol.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

Various mammalian cell culture systems can also be employed to express recombinant protein. Examples of mammalian expression systems include the COS-7 lines of monkey kidney fibroblasts, described-by Gluzman (Cell 23:175 (1981)), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

In a specific embodiment, constructs designed to express a portion of the extracellular domain of the Neutrokine-alpha (e.g., amino acid residues Ala-134 through Leu-285) are preferred. One of skill in the art would be able to use the polynucleotide and polypeptide sequences provided as SEQ ID NO:1 and SEQ ID NO:2, respectively, or SEQ ID NO:18 and SEQ ID NO:19, respectively, to design polynucleotide primers to generate such an expression construct.

In another embodiment, constructs designed to express the entire predicted extracellular domain of the Neutrokine-alpha (i.e., amino acid residues Gln-73 through Leu-285) are preferred. One of skill in the art would be able to use the polynucleotide and polypeptide sequences provided as SEQ ID NO:1 and SEQ ID NO:2, respectively, or SEQ ID NO:18 and SEQ ID NO:19, respectively, to design polynucleotide primers to generate such an expression construct.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., Neutrokine-alpha coding sequence), and/or to include genetic material (e.g., heterologous polynucleotide sequences) that is operably associated with Neutrokine-alpha polynucleotides of the invention, and which activates, alters, and/or amplifies endogenous Neutrokine-alpha polynucleotides. For example, techniques known in the art may be used to operably associate heterologous control regions (e.g., promoter and/or enhancer) and endogenous Neutrokine-alpha polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

The host cells described infra can be used in a conventional manner to produce the gene product encoded by the recombinant sequence. Alternatively, cell-free translation systems can also be employed to produce the polypeptides of the invention using RNAs derived from the DNA constructs of the present invention.

The polypeptide of the invention may be expressed or synthesized in a modified form, such as a fusion protein (comprising the polypeptide joined via a peptide bond to a heterologous protein sequence (of a different protein)), and may include not only secretion signals, but also additional heterologous functional regions. Such a fusion protein can be made by ligating polynucleotides of the invention and the desired nucleic acid sequence encoding the desired amino acid sequence to each other, by methods known in the art, in the proper reading frame, and expressing the fusion protein product by methods known in the art. Alternatively, such a fusion protein can be made by protein synthetic techniques, e.g., by use of a peptide synthesizer. Thus, for instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

In one embodiment, polynucleotides encoding Neutrokine-alpha and/or Neutrokine-alphaSV polypeptides of the invention may be fused to signal sequences which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the polypeptides of the invention may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides encoding Neutrokine-alpha and/or Neutrokine-alphaSV polypeptides of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that may be fused to a polypeptide of the invention in order to direct its secretion in mammalian cells include, but are not limited to, the MPIF-1 signal sequence (amino acids 1-21 of GenBank Accession number AAB51134), the stanniocalcin signal sequence (MLQNSAVLLLLVISASA, SEQ ID NO:45), and a consensus signal sequence (MPTWAWWLFLVLLLALWAPARG, SEQ ID NO:46). A suitable signal sequence that may be used in conjunction with baculoviral expression systems is the gp67 signal sequence, (amino acids 1-19 of GenBank Accession Number AAA72759).

A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5 has been fused with Fc portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995).

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Neutrokine-alpha protein includes naturally purified Neutrokine-alpha, Neutrokine-alpha of chemical synthetic procedures, and Neutrokine-alpha produced by recombinant techniques from a prokaryotic or eukaryotic host, including, for example, bacterial, yeast, higher plant, insect and mammalian cells (see, e.g., Example 3). Depending upon the host employed in a recombinant production procedure, the Neutrokine-alpha protein may be glycosylated or may be non-glycosylated. In addition, Neutrokine-alpha may also include an initial modified methionine residue, in some cases as a result of host-mediated processes.

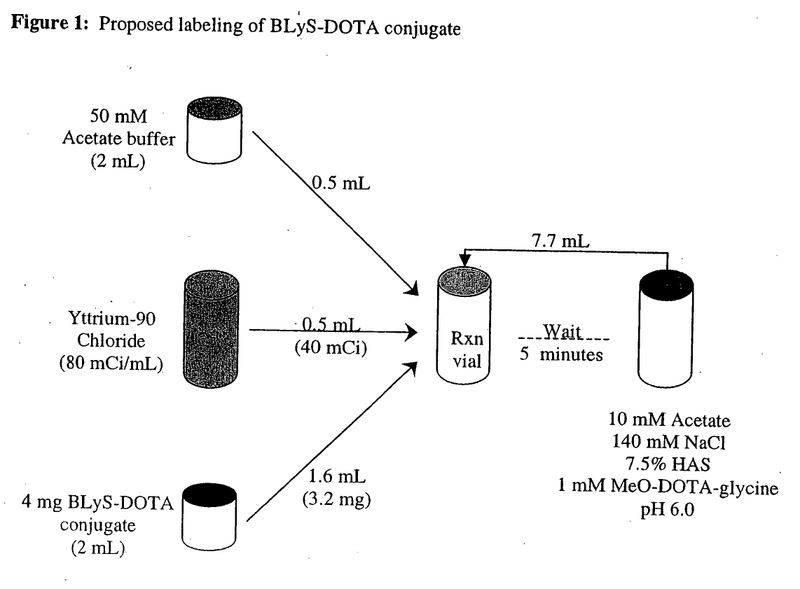

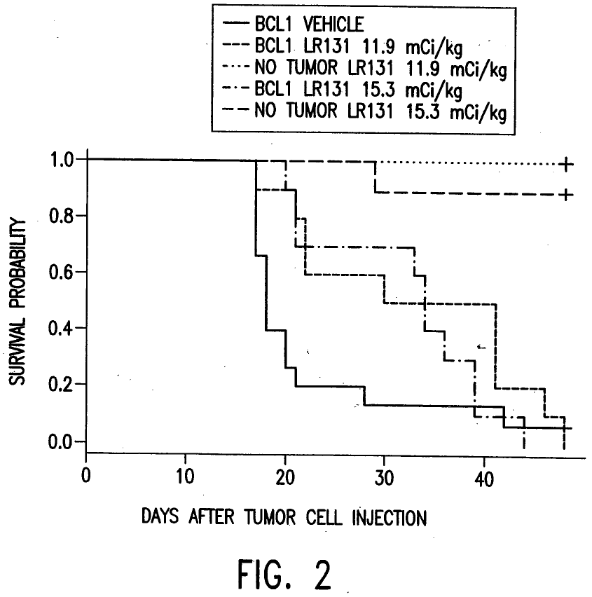

As one of skill in the art will appreciate, and as discussed above, the polypeptides of the present invention (e.g., the soluble mature form of human Neutrokine-alpha) can be fused to heterologous polypeptide sequences. For example, Neutrokine-alpha (including fragments or variants thereof), may be fused with the constant domain of immunoglobulins (IgA, IgE, IgG, IgM), or portions thereof (CH1, CH2, CH3, or any combination thereof and portions thereof, resulting in chimeric polypeptides. By way of another non-limiting example, polypeptides and/or antibodies of the present invention (including fragments or variants thereof) may be fused with albumin (including but not limited to recombinant human serum albumin or fragments or variants thereof (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)). In a preferred embodiment, Neutrokine-alpha polypeptides (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. In another preferred embodiment, Neutrokine-alpha polypeptides (including fragments or variants thereof) are fused with polypeptide fragments comprising, or alternatively consisting of, amino acid residues 1-x of human serum albumin, where x is an integer from 1 to 585 and the albumin fragment has human serum albumin activity. In another preferred embodiment, Neutrokine-alpha polypeptides (including fragments or variants thereof) are fused with polypeptide fragments comprising, or alternatively consisting of, amino acid residues 1-z of human serum albumin, where z is an integer from 369 to 419, as described in U.S. Pat. No. 5,766,883 herein incorporated by reference in its entirety. Neutrokine-alpha polypeptides p (including fragments or variants thereof) may be fused to either the N- or C-terminal end of the heterologous protein (e.g., immunoglobulin Fc polypeptide or human serum albumin polypeptide).

Such fusion proteins as those described above may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972- 897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+nitriloacetic acid-agarose column and histidine-tagged proteins can be selectively eluted with imidazole-containing buffers.

Neutrokine-alpha can be chemically synthesized using techniques known in the art (e.g., see Creighton, 1983, Proteins: Structures and Molecular Principles, W. H. Freeman & Co., N.Y.; and Hunkapiller, M., et al., Nature 310:105-111 (1984)). For example, a peptide corresponding to a fragment of the Neutrokine-alpha can be synthesized by use of a peptide synthesizer. Furthermore, if desired, nonclassical amino acids or chemical amino acid analogs can be introduced as a substitution or addition into the Neutrokine-alpha sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

Neutrokine-alpha can also be differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Additional post-translational modifications suitable for Neutrokine-alpha include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Neutrokine-alpha may also be modified with a detectable label, such as an enzymatic, fluorescent, radioisotopic, or affinity label to allow for detection and isolation of the protein.

The Neutrokine-alpha protein as used in the present invention is preferably provided in an isolated form, and preferably are substantially purified. By “isolated” is intended Neutrokine-alpha protein removed from its native environment. Thus, for example, an protein produced and/or contained within a recombinant host cell is considered isolated for purposes of the present invention. By way of example, a protein that is “substantially purified” is meant a protein that is substantially free from substances that limit its effect or produce undesired side effects. Usually, a substantially purified protein will be at least 90% pure. A recombinantly produced version of the Neutrokine-alpha protein can be substantially purified by the one-step method described in Smith and Johnson, Gene 67:31-40 (1988).

Neutrokine-alpha can be routinely produced, recovered and purified by methods known in the art (see, e.g., Example 5 and U.S. application Ser. No. 10/270,487 which is hereby incorporated by reference in its entirety). Methods that can be used to purify Neutrokine-alpha include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Also suitable are chemically modified derivatives of Neutrokine-alpha which may provide additional advantages such as increased solubility, stability, and in vivo or in vitro circulating time of the polypeptide, or decreased immunogenicity (see, e.g., U. S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The Neutrokine-alpha protein may be modified at random positions within the molecule, or at predetermined positions within the molecule and may include one, two, three or more attached chemical moieties.

The polymer may be of any molecular weight and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any, on biological activity, the ease in handling, the degree or lack of antigenicity, and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules or other chemical moieties should be attached to the protein with consideration of effects on functional or antigenic domains of the protein. Furthermore, the polyethylene glycol molecules or other chemical moieties should be attached to the protein with consideration of possible effects on the chelator molecule of said Neutrokine-alpha conjugate or Neutrokine-alpha complex. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include, for example, lysine residues and the N- terminal amino acid residues; those having a free carboxyl group may include aspartic acid residues, glutamic acid residues, and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of Neutrokine-alpha may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monomethoxy polyethylene glycol (MPEG) using tresylchloride (CISO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoroethanesulphonyl group.

Polyethylene glycol can also be attached to a protein using any of a number of different intervening linkers. For example, U.S. Pat. No. 5,612,460, the entire disclosure of which is incorporated herein by reference, discloses urethane linkers for connecting polyethylene glycol to a protein. Protein-polyethylene glycol conjugates wherein the polyethylene glycol is attached to the protein by a linker can also be produced by reaction of a protein with a compound such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to a protein are described in WO 98/32466, the entire disclosure of which is incorporated herein by reference. Pegylated Neutrokine-alpha produced using the reaction chemistries set out herein are included as being suitable within the scope of the present invention.

The number of polyethylene glycol moieties attached to each Neutrokine-alpha (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14 -16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

Neutrokine-alpha Conjugate:

Chelator and Association of Chelator with Neutrokine-alpha Protein

Chelator molecules that may be used in the Neutrokine-alpha complexes and Neutrokine-alpha conjugates of the present invention are known in the art. For example, see Subramanian, R. and Meares, C. F., “Bifunctional Chelating Agents for Radiometal-labeled monoclonal Antibodies,” in Cancer Imaging with Radiolabeled Antibodies (D. M. Goldenberg, Ed.) Kluwer Academic Publications, Boston; Saji, H., “Targeted delivery of radiolabeled imaging and therapeutic agents: bifunctional radiopharmaceuticals,” Crit. Rev. Ther. Drug Carrier Syst. 16:209-244 (1999); Srivastava S. C. and Mease R. C., “Progress in research on ligands, nuclides and techniques for labeling monoclonal antibodies,” Int. J. Rad. Appl. Instrum. B 18:589-603 (1991); and Liu, S. and Edwards, D. S., “Bifunctional chelators for therapeutic lanthanide radiopharmaceuticals,” Bioconjug. Chem. 12:7-34 (2001). Any chelator which can be covalently bound to said Neutrokine-alpha protein may be used according to the present invention. The chelator may further comprise a linker moiety that connects the chelating moiety to the Neutrokine-alpha protein.

In one embodiment, the chelator is an acyclic chelator such as diethylene triamine-N,N,N′,N″,N″-pentaacetic acid (DPTA), analogues of DPTA, and derivatives of DPTA. As non-limiting examples, the chelator may be 2-(p-isothiocyanatobenzyl)-6-methyldiethylenetriaminepentaacetic acid (1B4M-DPTA, also known as MX-DTPA), 2-methyl-6-(rho-nitrobenzyl)-1,4,7-triazaheptane-N,N,N′,N″,N″-pentaacetic acid (nitro-1B4M-DTPA or nitro-MX-DTPA); 2-(p-isothiocyanatobenzyl)-cyclohexyldiethylene-triaminepentaacetic acid (CHX-DTPA), or N-[2-amino-3-(rho-nitrophenyl)propyl]-trans-cyclohexane-1,2-diamine-N,N′,N″-pentaacetic acid (nitro-CHX-A-DTPA).

In another embodiment, the chelator is an acyclic terpyridine chelator such as 6,6″-bis[[N,N,N″,N″-tetra(carboxymethyl)amino]methyl]-4′-(3-amino-4-methoxyphenyl)-2,2′:6′,2″-terpyridine (TMT-amine).

In a specific embodiment, Neutrokine-alpha proteins of the invention are attached to macrocyclic chelators useful for conjugating radiometal ions, including but not limited to, ¹¹¹In, ¹⁷⁷Lu, ⁹⁰Y, ¹⁶⁶Ho, and ¹⁵³Sm, to polypeptides. In a specific embodiment, the radiometal ion which associates with the macrocyclic chelators attached to Neutrokine-alpha proteins of the invention is ¹¹¹n. In another preferred embodiment, the radiometal ion which associates with the macrocyclic chelator attached to Neutrokine-alpha proteins of the invention is ⁹⁰Y. In specific embodiments, the macrocyclic chelator is 1,4,7,10-tetraazacyclododecane-N,N′,N″,N′″-tetraacetic acid (DOTA). In other specific embodiments, the DOTA is attached to the Neutrokine-alpha and/or Neutrokine-alphaSV polypeptide of the invention via a linker molecule. Examples of linker molecules useful for conjugating DOTA to a polypeptide are commonly known in the art—see, for example, DeNardo et al., Clin. Cancer Res. 4(10):2483-90, 1998; Peterson et al., Bioconjug. Chem. 10(4):553-7, 1999; and Zimmerman et al., Nucl. Med. Biol. 26(8):943-50, 1999 which are hereby incorporated by reference in their entirety. In addition, U.S. Pat. Nos. 5,652,361 and 5,756,065, which disclose chelating agents that may be conjugated to antibodies, and methods for making and using them, are hereby incorporated by reference in their entireties. Though U.S. Pat. Nos. 5,652,361 and 5,756,065 focus on conjugating chelating agents to antibodies, one skilled in the art could readily adapt the method disclosed therein in order to conjugate chelating agents to other polypeptides.

Bifunctional chelators based on macrocyclic ligands in which conjugation is via an activated arm, or functional group, attached to the carbon backbone of the ligand can be employed as described by M. Moi et al., J. Amer. Chem. Soc. 49:2639 (1989) (2-p-nitrobenzyl-1,4,7,10-tetraazacyclododecane-N,N′,N″,N′″-tetraacetic acid); S. V. Deshpande et al., J. Nucl. Med. 31:473 (1990); G. Ruser et al., Bioconj. Chem. 1:345 (1990); C. J. Broan et al., J. C. S. Chem. Comm. 23:1739 (1990); and C. J. Anderson et al., J. Nucl. Med. 36:850 (1995).

In one embodiment, the chelator is a macrocyclic chelator, such as polyazamacrocyclic chelators, optionally containing one or more carboxy, amino, hydroxamate, phosphonate, or phosphate groups. In another embodiment, the chelator is a chelator selected from the group consisting of DOTA, analogues of DOTA, and derivatives of DOTA.

In one embodiment, suitable chelator molecules include DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), and THT (4′-(3-amino-4-methoxy-phenyl)-6,6″-bis(N′,N′-dicarboxymethyl-N-methylhydra zino)-2,2′:6′,2″-terpyridine), and analogs and derivatives thereof. See, e.g., Ohmono et al., J. Med. Chem. 35: 157-162 (1992); Kung et al., J. Nucl. Med. 25: 326-332 (1984); Jurisson et al., Chem. Rev. 93:1137-1156 (1993); and U.S. Pat. No. 5,367,080. Other suitable chelators include chelating agents disclosed in U.S. Pat. Nos. 4,647,447; 4,687,659; 4,885,363; EP-A-71564; W089/00557; and EP-A-232751.

In another embodiment, suitable macrocyclic carboxylic acid chelators which can be used in the present invention include 1,4,7,10-tetraazacyclododecane-N,N′,N″,N′″-tetraacetic acid (DOTA); 1,4,8,12-tetraazacyclolpentadecane-N,N′,N″,N′″-tetraacetic acid (15N4); 1,4,7-triazacyclononane-N,N′,N″-triacetic acid (9N3); 1,5,9-triazacyclododecane-N,N′,N″-triacetic acid (12N3); and 6-bromoacetamido-benzyl-1,4,8,11-tetraazacyclotetradecane-N,N′,N″,N″′-tetraacetic acid (BAT).

In another embodiment, the chelator is a chelator having the Formula I:

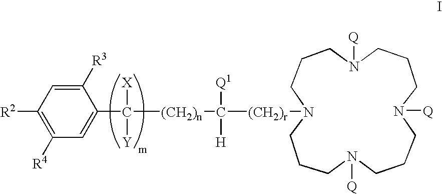

wherein

each Q is independently hydrogen or (CHR⁵)ₚCO₂R, preferably —CH₂—CO₂R, more preferably —CH₂COOH;

Q¹ is hydrogen or (CHR⁵)_{w}CO₂R, preferably CO₂R, more preferably COOH;

each R independently is hydrogen, benzyl or C₁-C₄ alkyl, preferably hydrogen or C₁₋₄ alkyl, more preferably hydrogen;

with the proviso that at least two of the sum of Q and Q¹ must be other than hydrogen;

each R⁵ independently is hydrogen, C₁-C₄ alkyl or —(C₁-C₂ alkyl)phenyl;

X and Y are each independently hydrogen or may be taken with an adjacent X and Y to form an additional carbon-carbon bond;

n is 0 or 1, preferably 0;

m is an integer from 0 to 10 inclusive, preferably 0 to 1, more preferably 0;

p is 1 or 2, preferably 1;

r is 0 or 1, preferably 0;

w is 0 or 1, preferably 0;

with the proviso that n is only 1 when X and/or Y form an additional carbon-carbon bond, and the sum of r and w is 0 or 1;

R2 is selected from the group consisting of hydrogen, nitro, amino, isothiocyanato, semicarbazido, thiosemicarbazido, maleimido, bromoacetamido and carboxyl, preferably hydrogen or isothiocyanato;

R³ is selected from the group consisting of C₁-C₄ alkoxy, —OCH₂CO₂H, hydroxy and hydrogen, preferably C₁-C₄ alkoxy, more preferably methoxy;

R⁴ is selected from the group consisting of hydrogen, nitro, amino, isothiocyanato, semicarbazido, thiosemicarbazido, maleimido, bromoacetamido and carboxyl, preferably hydrogen or isothiocyanato;

with the proviso that R and R4 cannot both be hydrogen but one of R² and R⁴ must be hydrogen; or a pharmaceutically acceptable salt thereof. Such a compound is described in U.S. Pat. No. 5,652,361.

With reference to the chelator of Formula I, it is understood that the chelator portion of the Neutrokine-alpha conjugate of the present invention is formed from a molecule of Formula I. Accordingly, in one embodiment, the Neutrokine-alpha protein is bonded to an appropriate functional group on a compound of Formula I. For example, in one embodiment, the functional group designated R² is reacted with a Neutrokine-alpha protein to form a Neutrokine-alpha conjugate. By way of example, in one embodiment, R² is an isothiocyanato and reacts with a hydroxyl group on a serine residue of a Neutrokine-alpha protein. In another embodiment, the functional group designated R⁴ is reacted with a Neutrokine-alpha protein to form a Neutrokine-alpha conjugate.

In another specific embodiment, the chelator of said conjugate is formed from α-(5-isothiocyanato-2-methoxyphenyl)-1,4,7,10-tetraaza-cyclododecane-1,4,7,10-tetraacetic acid (MeO-DOTA-NCS), which has the structure of Formula II:

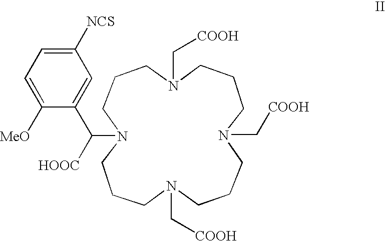

wherein “NCS” denotes a isothiocyanato group. A pharmaceutically acceptable salt or ester of α-(5-isothiocyanato-2-methoxyphenyl)-1,4,7,10-tetraazacyclododecane-1,4,7,10-tetraacetic acid may also be used.

When forming the Neutrokine-alpha conjugate using a chelator as described above, the Neutrokine-alpha protein reacts with a functional group on the chelator to form a covalent bond and thus form the conjugate. With reference to a chelator according to Formula I, the Neutrokine-alpha protein will preferentially react with a functional group selected from the group consisting of amino, isothiocyanato, semicarbazido, thiosemicarbazido, maleimido, bromoacetamido and carboxyl. Thus, the Neutrokine-alpha protein will preferentially form a bond with a functional group of either R² or R⁴. Thus, in accordance with the description of a chelator according to Formula I, in one embodiment, a Neutrokine-alpha conjugate has the structure of Formula III:

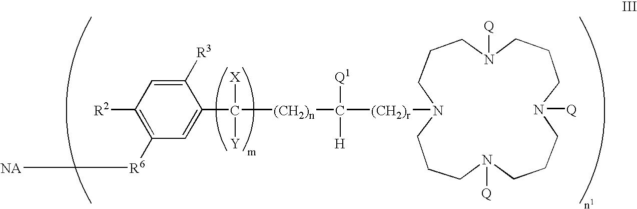

wherein n¹ is an integer from 1 to about 30, in another embodiment from 1 to about 20, in yet another embodiment 1, 2, 3, 4, 5, 6, 7, 8, 9, or 10, in yet another embodiment from about 1 to about 5, or in another embodiment about 1;

Q, Q¹, R, R², R³, R⁵, X, Y, n, m, p, r, and ware defined as above; and

NA is a Neutrokine-alpha protein;

wherein R⁶ is a functional group resulting from the reaction of an amino acid residue of NA with a functional group selected from the group defined for R⁴ above, i.e., consisting of amino, isothiocyanato, semicarbazido, thiosemicarbazido, maleimido, bromoacetamido and carboxyl, preferably isothiocyanato.

In one embodiment, the Neutrokine-alpha conjugate of the present invention has the structure of Formula III, wherein said NA is a mature, soluble Neutrokine-alpha protein or a fragment or variant thereof. In another embodiment, the Neutrokine-alpha conjugate of the present invention has the structure of Formula III, wherein said NA is a human mature, soluble Neutrokine-alpha protein or a fragment or variant thereof. In another embodiment, the Neutrokine-alpha conjugate of the present invention has the structure of Formula III, wherein said NA comprises, or alternatively consists of the sequence shown in Table 2 or a fragment or variant thereof.

With reference to a chelator according to Formula II, the Neutrokine-alpha protein, in one embodiment, reacts with an isothiocyanato group of the chelator. Thus, the Neutrokine-alpha protein, in one embodiment, forms a bond with the isothiocyanato group to form a thiocarbamate group or a thiourea group, preferably a thiourea group. Thus, in accordance with the description of a chelator according to Formula II, a particular Neutrokine-alpha conjugate has the structure of Formula IV:

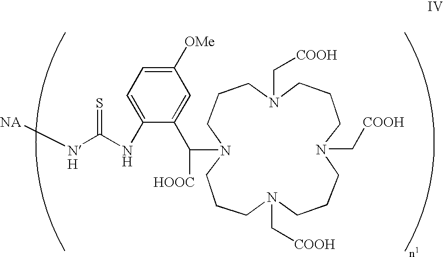

or a pharmaceutically acceptable salt thereof, wherein NA is a Neutrokine-alpha protein;

n¹ is an integer from 1 to about 30, preferably from about 1 to about 20, preferably from about 1 to about 5, most preferably about 1; and

N′ is a nitrogen atom from an amino acid residue, preferably the N-terminal amino acid residue or a lysine residue, of the Neutrokine-alpha protein.

In one embodiment, the Neutrokine-alpha protein of Formula IV is any Neutrokine-alpha protein as described herein. In one embodiment, the Neutrokine-alpha conjugate of the present invention has the structure of Formula IV, wherein said NA is a mature, soluble Neutrokine-alpha protein or a fragment or variant thereof. In another embodiment, the Neutrokine-alpha conjugate of the present invention has the structure of Formula IV, wherein said NA is a human mature, soluble Neutrokine-alpha protein (i.e., amino acids 134-285 of SEQ ID NO:2) or a fragment or variant thereof. In another embodiment, the Neutrokine-alpha conjugate of the present invention has the structure of Formula IV, wherein said NA comprises the sequence shown in Table 1 or Table 2 or a fragment or variant thereof.

It is understood that other functional groups, as described above, may be used to link the chelator to the Neutrokine-alpha protein.

As used herein, “pharmaceutically acceptable salt” means any salt of a compound of Formulae I-IV which is sufficiently non-toxic to be useful in therapy or diagnosis of mammals. Thus, the salts are useful in accordance with this invention. Representative of those salts, which are formed by standard reactions, from both organic and inorganic sources include, for example, sulfuric, hydrochloric, phosphoric, acetic, succinic, citric, lactic, maleic, fumaric, palmitic, cholic, palmoic, mucic, glutamic, d-camphoric, glutaric, glycolic, phthalic, tartaric, formic, lauric, steric, salicylic, methanesulfonic, benzenesulfonic, sorbic, picric, benzoic, cinnamic acids and other suitable acids. Also included are salts formed by standard reactions from both organic and inorganic sources such as ammonium, alkali metal ions, alkaline earth metal ions, and other similar ions. Particularly preferred are the salts of a chelator where the salt is potassium, sodium, ammonium, or mixtures thereof.

Of course, the free acid of the compounds and conjugates of Formulae I-IV may be used, also the protonated form of the compounds, for example when the carboxylate is protonated and/or the nitrogen atoms are protonated, e.g., when the HCl salt is formed.

The Neutrokine-alpha conjugate may contain more than an average of one chelator molecule per monomer of Neutrokine-alpha. In one embodiment, the Neutrokine-alpha conjugate contains about 1, 2, 3, 4, 5, 6, 7, 8, 9, 10, 15, 20, 25, 30, or 35 chelator molecules per monomer of Neutrokine-alpha. In another embodiment, the Neutrokine-alpha conjugate contains about 1, 2, 3, 4, 5, 6, 7, 8, 9, or 10 chelator molecules per monomer of Neutrokine-alpha protein. In another embodiment, the Neutrokine-alpha conjugate contains about 1, 2, 3, 4, or 5 chelator molecules per monomer of Neutrokine-alpha protein. In another embodiment, the Neutrokine-alpha conjugate contains an average of about 1 chelator molecule per monomer of Neutrokine-alpha. In another embodiment, the Neutrokine-alpha conjugate contains about 1.1 molecules per monomer of Neutrokine-alpha protein.

The Neutrokine-alpha conjugate may contain more than an average of one chelator molecule per monomer of Neutrokine-alpha. In one embodiment, the Neutrokine-alpha conjugate contains at least 1, 2, 3, 4, 5, 6, 7, 8, 9, 10, 15, 20, 25, 30, or 35 chelator molecules per monomer of Neutrokine-alpha. In another embodiment, the Neutrokine-alpha conjugate contains at least 1, 2, 3, 4, 5, 6, 7, 8, 9, or 10 chelator molecules per monomer of Neutrokine-alpha protein. In another embodiment, the Neutrokine-alpha conjugate contains at least 1, 2, 3, 4, or 5 chelator molecules per monomer of Neutrokine-alpha protein. In another embodiment, the Neutrokine-alpha conjugate contains an average of at least 1 chelator molecule per monomer of Neutrokine-alpha.

The Neutrokine-alpha conjugate, as described above, comprises a Neutrokine-alpha protein and a chelator molecule covalently bonded together. The covalent bond between the protein and chelator is formed between an atom of the protein and an atom of the chelator. The atom of the protein that forms the covalent bond can be any suitable atom of the protein, such as a carbon, nitrogen, oxygen, and sulfur. Certain functional groups on the protein are preferred to form the covalent bond with the chelator. Such groups include an amino group at the terminus of the protein; a carboxy group at the terminus of the protein; an amino group on a lysine residue; a carboxy group on an aspartame or glutamate residue; a guanidino group on an arginine residue; a thiol group on a cysteine residue; a hydroxy group on a serine residue or threonine residue; a imidazole group of a histidine residue; a hydroxy group of a tyrosine; a hydroxy group of a tryptophan group; and an amide group of an asparagine or glutamine residue.

In one embodiment, the chelator is bonded to a lysine residue of the Neutrokine-alpha protein. In another embodiment, the chelator is bonded to one or more residues selected from the group consisting of LYS 160, LYS 173, LYS181, LYS184, LYS188, LYS204, LYS215, LYS216, LYS252, and LYS283.

In another embodiment, the chelator is bonded to ALA134.

In another embodiment, the chelator is bonded to an N-terminal amino acid of the Neutrokine-alpha protein.

In another embodiment, the chelator is bonded to the C-terminus amino acid of the Neutrokine-alpha protein.

Accordingly, in one embodiment, the Neutrokine-alpha conjugate of the present invention is a molecule according to Formula IV, wherein the chelator molecule is bonded to LYS160. In another embodiment, the Neutrokine-alpha conjugate of the present invention is a molecule according to Formula IV, wherein the chelator molecule is bonded to LYS173. In another embodiment, the Neutrokine-alpha conjugate of the present invention is a molecule according to Formula IV, wherein the chelator molecule is bonded to LYS181. In another embodiment, the Neutrokine-alpha conjugate of the present invention is a molecule according to Formula IV, wherein the chelator molecule is bonded to LYS 184. In another embodiment, the Neutrokine-alpha conjugate of the present invention is a molecule according to Formula IV, wherein the chelator molecule is bonded to LYS188. In another embodiment, the Neutrokine-alpha conjugate of the present invention is a molecule according to Formula IV, wherein the chelator molecule is bonded to LYS204. In another embodiment, the Neutrokine-alpha conjugate of the present invention is a molecule according to Formula IV, wherein the chelator molecule is bonded to LYS215. In another embodiment, the Neutrokine-alpha conjugate of the present invention is a molecule according to Formula IV, wherein the chelator molecule is bonded to LYS216. In another embodiment, the Neutrokine-alpha conjugate of the present invention is a molecule according to Formula IV, wherein the chelator molecule is bonded to LYS252. In another embodiment, the Neutrokine-alpha conjugate of the present invention is a molecule according to Formula IV, wherein the chelator molecule is bonded to LYS283. In another embodiment, the Neutrokine-alpha conjugate of the present invention is a molecule according to Formula IV, wherein the chelator molecule is bonded to ALA134.

In a further embodiment, the Neutrokine-alpha conjugate, existing as a trimer, has a differential conjugation pattern. That is, each monomer of the trimer has a chelator molecule attached to a different amino acid. By way of example, in one embodiment, a Neutrokine-alpha conjugate trimer has one monomer with a chelator attached to LYS252, has another monomer with a chelator attached to LYS 283, and a third monomer with a chelator attached to ALA134.

As described earlier, Neutrokine-alpha can exist as a monomer protein or as multiple subunits associated together. In one embodiment, the Neutrokine-alpha conjugate is any of the specific embodiments described above wherein the conjugate is in the monomer form. In another embodiment, the Neutrokine-alpha conjugate is any of the specific embodiments described above wherein the conjugate is in the trimer form.

Neutrokine-alpha Complex

An additional embodiment of the present invention is directed to a Neutrokine-alpha complex comprising a Neutrokine-alpha conjugate and a metal ion, wherein said metal ion is associated with the chelator moiety of said Neutrokine-alpha conjugate. Specific embodiments of a Neutrokine-alpha complex of the invention include a complex comprising a Neutrokine-alpha conjugate, as set forth herein, and a metal ion. Herein “metal ion” refers to any ion that can associate with the chelator moiety of the invention as described herein.

Metal Ion

Any metal ion that associates with the chelating moiety of said Neutrokine-alpha conjugate may be used. Such a metal ion includes a metal ion selected from the group consisting of Ac, Ag, At, Au, Bi, Ce, Co, Cr, Cu, Dy, Er, Eu, Fe, Ga, Gd, Hg, Ho, In, La, Lu, Mn, Mo, Nd, Ni, Os, Pb, Pd, Pm, Pr, Pt, Rb, Re, Rh, Ru, Sb, Sc, Si, Sm, Sn, Sr, Th, Tc, Ti, Tm, V, W, Y, and Yb.

In one embodiment of the present invention, the metal ion of the complex is a radionuclide. Radionuclides useful in the Neutrokine-alpha complex are known in the art. The radionuclides useful in the present invention include, but are not limited to, gamma-emitters, positron-emitters, x- ray emitters, fluorescence-emitters, beta-emitters, alpha-emitters, auguer electron emitters, and electron and neutron-capturing agents. In one embodiment, a gamma-emitter, positron-emitter, x-ray emitter, and/or fluorescence-emitter is used for localization and/or therapy. In another embodiment, a beta-emitter, alpha-emitter, or an electron-capturing or neutron-capturing agent, such as boron or uranium, is used for therapy.

The radionuclide used in the complex of the present invention may be suitable for therapeutic, diagnostic, or both therapeutic and diagnostic purposes. Examples of appropriate metals include Ag, At, Au, Bi, Cu, Ga, Ho, In, Lu, Pb, Pd, Pm, Pr, Rb, Re, Rh, Sc, Sr, Tc, Tl, Y, and Yb. Examples of the radionuclide used for diagnostic purposes include Fe, Gd, ¹¹¹In, ⁶⁷Ga, or ⁶⁸Ga. In another embodiment, the radionuclide used for diagnostic purposes is ¹¹¹In, or ⁶⁷Ga. Examples of the radionuclide used for therapeutic purposes include ¹⁶⁶Ho, ¹⁶⁵Dy, ⁹⁰Y ^{115m}In, ⁵²Fe, or ⁷²Ga. In one embodiment, the radionuclide used for diagnostic purposes is ¹⁶⁶Ho or ⁹⁰Y. Examples of the radionuclides used for both therapeutic and diagnostic purposes include ¹⁵³Sm, ¹⁷⁷Lu, ¹⁵⁹Gd, ¹⁷⁵Yb, or ⁴⁷Sc. In one embodiment, the radionuclide is ¹⁵³Sm, ¹⁷⁷Lu, ¹⁷⁵Yb, or ¹⁵⁹Gd.

Preferred metal radionuclides include, but are not limited to, ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ⁴⁷Sc, ⁶⁷Ga, ⁵¹Cr, ^{177m}Sn, ⁶⁷Cu, ¹⁶⁷Tm, ⁹⁵Ru, ¹⁸⁸Re, ¹⁷⁷Lu, ¹⁹⁹Au, ²⁰³Pb and ¹⁴¹Ce.

In a particular embodiment, the metal ion of the Neutrokine-alpha complex is selected from the group consisting of ⁹⁰Y, ¹¹¹In, ¹⁷⁷Lu, ¹⁶⁶Ho, ²¹⁵Bi, and ²²⁵Ac.

Moreover, according to the present invention, γ-emitting radionuclides, such as ^{99m}Tc, ¹¹¹In, ⁶⁷Ga, and ¹⁶⁹Yb may be used for diagnostic imaging, while complexes of β-emitters, such as ⁶⁷Cu, ¹¹¹Ag, ¹⁸⁶Re, and ⁹⁰Y are useful for the applications in tumor therapy. Also other useful radionuclides include γ-emitters, such as ^{99m}Tc, ¹¹¹In, ⁶⁷Ga, and ¹⁶⁹Yb, and β-emitters, such as ⁶⁷Cu, ¹¹¹Ag, ¹⁸⁶Re, ¹⁸⁸Re and ⁹⁰Y, as well as other radionuclides of interest such as ²¹¹At, ²¹²Bi, ¹⁷⁷Lu, ⁸⁶Rb, ¹⁰⁵Rh, ¹⁵³Sm, ¹⁹⁸Au, ¹⁴⁹Pm, ⁸⁵Sr, ¹⁴²Pr, ²¹⁴Pb, ¹⁰⁹Pd, ¹⁶⁶Ho, ²⁰⁸Tl, and ⁴⁴Sc.

In another embodiment, paramagnetic metal ions that may be used according to the present invention include ions of transition and lanthanide metal, such as metals having atomic numbers of 21-29, 42, 43, 44, or 57-71, in particular ions of Cr, V, Mn, Fe, Co, Ni, Cu, La, Ce, Pr, Nd, Pm, Sm, Eu, Gd, Tb, Dy, Ho, Er, Tm, Yb, and Lu. The paramagnetic metals used in the composition for magnetic resonance imaging include the elements having atomic numbers of 22 to 29, 42, 44 and 58-70.

In another embodiment, fluorescent metal ions that may be used according to the present invention include lanthanides, in particular La, Ce, Pr, Nd, Pm, Sm, Eu, Gd, Th, Dy, Ho, Er, Tm, Yb, and Lu.

In another embodiment, heavy metal-containing reporters that may be used according to the present invention may include atoms of Mo, Bi, Si, and W.

In an additional embodiment, the Neutrokine-alpha complex of the invention comprises a metal ion selected from the group consisting of ⁹⁰Y, ¹¹¹In, ¹⁷⁷Lu, ¹⁶⁶Ho, ²¹⁵Bi, and ²²⁵Ac, and a conjugate according to Formula IV. In another embodiment, the Neutrokine-alpha complex of the invention comprises a metal ion selected from the group consisting of ⁹⁰Y, ¹¹¹In, ¹⁷⁷Lu, ¹⁶⁶Ho, ²¹⁵Bi, and ²²⁵Ac, and a conjugate according to Formula IV, wherein said Neutrokine-alpha protein comprises, or alternatively consists of, amino acids 134-285 of the sequence shown in Table 1 or a fragment or variant thereof.

In an additional embodiment, the Neutrokine-alpha complex of the invention comprises ⁹⁰Y and a conjugate according to Formula IV, wherein said Neutrokine-alpha protein comprises, or alternatively consists of, amino acids 134-285 of the sequence shown in Table 1 (SEQ ID NO:2).

In an additional embodiment, the Neutrokine-alpha complex of the invention comprises ¹¹¹In and a conjugate according to Formula IV, wherein said Neutrokine-alpha protein comprises, or alternatively consists of, amino acids 134-285 of the sequence shown in Table 1 (SEQ ID NO:2).

In an additional embodiment, the Neutrokine-alpha complex of the invention comprises ¹⁷⁷Lu and a conjugate according to Formula IV, wherein said Neutrokine-alpha protein comprises, or alternatively consists of, amino acids 134-285 of the sequence shown in Table 1 (SEQ ID NO:2).

In an additional embodiment, the Neutrokine-alpha complex of the invention comprises ¹⁶⁶Ho and a conjugate according to Formula IV, wherein said Neutrokine-alpha protein comprises, or alternatively consists of, amino acids 134-285 of the sequence shown in Table 1 (SEQ ID NO:2).

In an additional embodiment, the Neutrokine-alpha complex of the invention comprises ²¹⁵Bi and a conjugate according to Formula IV, wherein said Neutrokine-alpha protein comprises, or alternatively consists of, amino acids 134-285 of the sequence shown in Table 1 (SEQ ID NO:2).

In an additional embodiment, the Neutrokine-alpha complex of the invention comprises ¹⁶⁶Ho and a conjugate according to Formula IV, wherein said Neutrokine-alpha protein comprises, or alternatively consists of, amino acids 134-285 of the sequence shown in Table 1 (SEQ ID NO:2).

Method of Preparing Conjugate

An additional embodiment of the present invention is directed to a method of preparing a Neutrokine-alpha conjugate. The general procedure for preparing a Neutrokine-alpha conjugate comprises reacting a Neutrokine-alpha protein with a chelator. A suitable chelator will be able to react with the Neutrokine-alpha protein to form a covalent bond between the chelator and the Neutrokine-alpha protein.

Any chelator which is able to form a covalent bond with the Neutrokine-alpha protein may be used to form the Neutrokine-alpha conjugate. Such chelators are described herein or are otherwise known in the art, as are methods for preparing and/or attaching such chelators.

In one embodiment, a chelator to be used in the method according to the present invention is an activated chelator. The activated chelator contains a functional group that will readily react with a functional group on the protein, thereby forming a covalent bond between the protein and the chelator. Such functional groups are known in the art.

A suitable reactive functional group is a group that will react directly with carboxy, aldehyde, amine, alcohol, or sulfhydryl group on the Neutrokine-alpha protein. Such groups include, for example, active halogen containing groups including, for example, chloromethylphenyl groups and chloroacetyl (ClCH₂C(═O)—) groups; activated 2-(leaving group substituted)-ethylsulfonyl and ethylcarbonyl groups such as 2-chloroethylsulfonyl and 2-chloroethylcarbonyl; vinylsulfonyl; vinylcarbonyl; epoxy; isocyanato; isothiocyanato; aldehyde; aziridine; succinimidoxycarbonyl; activated acyl groups such as carboxylic acid halides; and mixed anhydrides.

A chelator which can be used in the present method includes, for example, any specific chelator as described above for the Neutrokine-alpha conjugate. Moreover, in another embodiment, the chelator used in the method of the invention is a chelator which can be used to prepare any specific Neutrokine-alpha conjugate as described above. In one embodiment, the chelator is an activated chelator selected from the group consisting of DOTA, analogues of DOTA, and derivatives of DOTA, wherein each of said DOTA, analogues of DOTA, and derivatives of DOTA contains a suitable activating group which enables the chelator molecule to be covalently bonded to the Neutrokine-alpha protein.

In one embodiment, the activated chelator for use in preparing a conjugate according to the present invention is an activated chelator having the Formula:

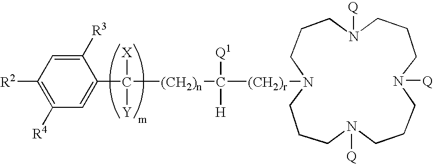

wherein

each Q is independently hydrogen or (CHR⁵)ₚCO₂R, preferably —CH₂—CO₂R, more preferably —CH₂CO₂H;

Q¹ is hydrogen or (CHR⁵)_{w}CO₂R, preferably CO₂R, more preferably COOH;

each R independently is hydrogen, benzyl or C₁-C₄ alkyl; preferably hydrogen or C₁-C₄ alkyl, more preferably hydrogen;

with the proviso that at least two of the sum of Q and Q¹ must be other than hydrogen;

each R⁵ independently is hydrogen, C₁-C₄ alkyl or —(C₁-C₂alkyl)phenyl;

X and Y are each independently hydrogen or may be taken with an adjacent X and Y to form an additional carbon-carbon bond;

n is 0 or 1; preferably 0

m is an integer from 0 to 10 inclusive, preferably 0 to 1, more preferably 0;

p is 1 or 2, preferably 1;

r is 0 or 1, preferably 0;

w is 0 or 1, preferably 0;

with the proviso that n is only 1 when X and/or Y form an additional carbon-carbon bond, and the sum of r and w is 0 or 1;

R² is selected from the group consisting of hydrogen, nitro, amino, isothiocyanato, semicarbazido, thiosemicarbazido, maleimido, bromoacetamido and carboxyl, preferably hydrogen or isothiocyanato;

R³ is selected from the group consisting of C₁-C₄ alkoxy, —OCH₂CO₂H, hydroxy and hydrogen, preferably C₁-C₄ alkoxy, more preferably methoxy;

R⁴ is selected from the group consisting of hydrogen, nitro, amino, isothiocyanato, semicarbazido, thiosemicarbazido, maleimido, bromoacetamido and carboxyl, preferably hydrogen or isothiocyanato;

with the proviso that R² and R⁴ cannot both be hydrogen but one of R² and R⁴ must be hydrogen; or a pharmaceutically acceptable salt thereof. Such a compound is described in U.S. Pat. No. 5,652,361.

In another embodiment, the activated chelator for use in preparing a conjugate according to the present invention is α-(5-isothiocyanato-2-methoxyphenyl)-1,4,7,10-tetraazacyclododecane-1,4,7,10-tetraacetic acid, which has the following structure:

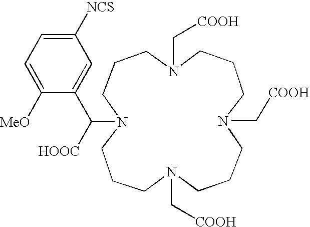

wherein “NCS” denotes an isothiocyanato group, and which is also known as MeO-DOTA-NCS. A salt or ester of α-(5-isothiocyanato-2-methoxyphenyl)-1,4,7,10-tetraazacyclododecane-1,4,7,10-tetraacetic acid may also be used.

In a specific embodiment of the invention, the Neutrokine-alpha protein is trimer of Neutrokine-alpha monomeric subunits wherein each Neutrokine-alpha subunit consists of amino acids 134-285 of human Neutrokine-alpha and having the sequence listed in Table 1 (SEQ ID NO:2). In additional specific embodiments, the Neutrokine-alpha protein used in the method of the invention is any one of the specific Neutrokine-alpha proteins described above. In additional specific embodiments, the Neutrokine-alpha conjugate prepared according to the present method is any one of the specific Neutrokine-alpha conjugates described above. In particular, the Neutrokine-alpha protein used in the present method is in the form of a trimer, as described above.

In one embodiment, the method of preparing a Neutrokine-alpha conjugate comprises mixing, agitating, or preparing a solution, said solution comprising a Neutrokine-alpha protein and a chelator. In an additional embodiment, the method of preparing a Neutrokine-alpha conjugate comprises mixing, agitating, or preparing a solution comprising a Neutrokine-alpha protein, a chelator, citrate buffer, HEPES buffer, and sterile water. In an additional embodiment, the solution further comprises NaOH. In an additional embodiment, the solution has a pH of about 8.5.

In an additional embodiment, the method of preparing the conjugate comprises a) mixing, agitating, or preparing a solution comprising a Neutrokine-alpha protein and a chelator, at a temperature of about 0° C. to about 50° C. for about 0.5 hours to about 24 hours, wherein said solution has a pH of about 8.0 to about 9.0; and b) optionally adding a quenching agent.

In one embodiment of the above process, the solution is mixed, agitated or prepared at a temperature of about 0° C. to about 50° C. In another embodiment of the above process, the solution is mixed, agitated or prepared at a temperature of about 20° C. to about 30° C. The temperature at which the solution is mixed, agitated, or prepared can be about 0, 5, 10, 15, 20, 25, 20, 35, 40, 45, or 50° C.

Any suitable chelator as described above may be used in an embodiment of the present process. Chelators as described herein may be purchased for example, from Dow Chemical Company, (Midland, Mich.).

When making a Neutrokine-alpha conjugate it is important to consider the ratio of the number of moles of chelator molecules in the reaction compared to the number of moles of sites to which the chelator molecule may attach in the Neutrokine-alpha protein (herein referred to a the molar ratio of chelator to chelator bonding sites). The molar ratio of chelator to chelator bonding sites used in the preparation of the conjugate can vary depending on the number of chelator bonding sites in the Neutrokine-alpha protein component of the Neutrokine-alpha conjugate. For example, if the Neutrokine-alpha protein component is a trimer of Neutrokine alpha monomers, wherein each monomer consists of the Neutrokine-alpha protein of SEQ ID NO:3, and the chelator is bonding to either the N terminus or to Lysine residues in the Neutrokine-alpha trimer, and it is desired that the Neutrokine-alpha conjugate product comprises, on average, 3 chelator molecules on the trimeric Neutrokine-alpha protein, it would be desirable to use a molar ratio of chelator to chelator binding sites in Neutrokine-alpha protein between 12:1 and 10:1 in the reaction (see, e.g., Example 5).

In specific embodiments, molar ratio of chelator to chelator binding sites in Neutrokine-alpha protein is less than or equal to 1000:1. In other specific embodiments, molar ratio of chelator to chelator binding sites in Neutrokine-alpha protein is less than or equal to 100:1. In preferred embodiments, molar ratio of chelator to chelator binding sites in Neutrokine-alphaprotein is 20:1, 15:1, 12:1, 11:1, 10:1 or 5:1.

In the final Neutrokine-alpha conjugate product, the molar ratio of chelator to Neutrokine-alpha protein monomer can be from about 10:1 to about 1:10. In one embodiment, the molar ratio of chelator to Neutrokine-alpha protein monomer may be 5:1 or about 1:5; In a preferred embodiment, from about 1:3 to about 3:1. In another preferred embodiment, the molar ratio of chelator to Neutrokine-alpha protein monomer in the final Neutrokine-alpha conjugate product, is about 1:1. In another embodiment, the molar ration of chelator to Neutrokine-alpha protein monomer in the final conjugate product is about 1.1:1. The molar ratio of chelator to Neutrokine-alpha protein monomers refers to the ratio of the number of chelator molecules to the number of Neutrokine-alpha protein molecules (monomers) in the product. As is known in the art, Neutrokine-alpha proteins can form bound oligomers, for example trimers. Thus, according to the present invention, each Neutrokine-alpha trimer is equal to three Neutrokine-alpha protein monomers when calculating the ratio of chelator to Neutrokine-alpha protein monomers.

When preparing a Neutrokine-alpha conjugate, the solution can be mixed, agitated, or allowed to stand for a variable number of hours, depending upon a number of variables, such factors including the identity of the chelator, the identity of the Neutrokine-alpha protein, the temperature of the reaction, the pH, the identity of the buffer, the molar ratio of the reactants, the purity of the available reagents, the presence of a catalyst or activator, and other factors which would be evident to, and routinely manipulated by, one of skill in the art to achieve a desired result. In one embodiment of the above process, the solution comprising a Neutrokine-alpha protein and a chelator is mixed, agitated, or allowed to stand for about 0.5 hours to about 24 hours. In another embodiment, the solution is mixed, agitated, or allowed to stand for about 1 hour to about 20 hours. In another embodiment, the solution is mixed, agitated, or allowed to stand for about 2 hour to about 10 hours. In another embodiment, the solution is mixed, agitated, or allowed to stand for about 3 hour to about 5 hours. In another embodiment, the solution is mixed, agitated, or allowed to stand for about 4 hours. In other embodiments, the solution is mixed, agitated, or allowed to stand for about 1, 2, 3, 4, 5, 6, 7, 8, 9, 10, 11, 12, 13, 14, 15, 16, 17, 18, 19, 20, 21, 22, 23, or 24 hours.

The pH of the solution used to prepare the Neutrokine-alpha conjugate can vary. In one embodiment, the pH is about 6, 7, 8, 9, or 10. In another embodiment, said solution has a pH of about 7 to about 10. In another embodiment, said solution has a pH of about 7.5 to about 9.5. In another embodiment, said solution has a pH of about 8 to about 9. In another embodiment, said solution has a pH of about 8.5.

The solution used to prepare the Neutrokine-alpha conjugate may further comprise a buffer. Buffers are well-known in the art and may be routinely applied to maintain the desired pH of the solutions used in making and/or using the Neutrokine-alpha conjugates and/or Neutrokine-alpha complexes of the invention. Suitable buffers for use in the preparation of a Neutrokine-alpha include, for example, those described below for the method of preparing a Neutrokine-alpha conjugate. In one embodiment, the buffer is a citrate buffer or an acetate buffer. In another embodiment, the buffer includes an acetate buffer having a concentration of about 1 to about 50 mM and having a NaCl concentration of about 1 to about 500 mM. In another embodiment, the buffer includes an acetate buffer having a concentration of about 10 mM and having a NaCl concentration of about 140 mM. Suitable acetate buffers include acetate buffers having a concentration of about 1, 20, 25, 50, 75, 100, 200, 250, 300, 400, or 500 mM. Suitable buffers and solutions include those having a NaCl concentration of about 1, 50, 75, 100, 125, 140, 150, 175, 200, 225, 250, 275, 300, 350, 400, 450, or 500 mM. An additional suitable buffer is a HEPES buffer, in particular a HEPES buffer having a concentration of about 10, 20, 30, 40, 50, 60, 70, 800, 90, 100, 200, 300, 400, or 500 mM. In an additional embodiment, the solution comprises a HEPES buffer having a concentration of about 50 mM. In another embodiment, a citrate buffer is used, having a concentration of about 1 to about 100 mM, or about 1, 2, 5, 7, 10 15, or 20 mM.

The concentration of the Neutrokine-alpha protein used in the method of the invention may vary. In one embodiment, the concentration of Neutrokine-alpha protein in the solution is from about 0.1 mg/mL to about 10 mg/mL. In another embodiment, the Neutrokine-alpha concentration is about 0.5 mg/mL to about 5 mg/mL. In other specific embodiments, the Neutrokine-alpha concentration is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mg/mL. In another specific embodiment, the concentration of Neutrokine-alpha protein in the solution is about 1 mg/mL to about 2 mg/mL.

A reagent which facilitates the formation of the conjugate can also be used according to the methods of the invention. Such reagents typically activate a chelator so that it reacts more readily with a protein. Alternatively, the reagent may activate the protein to react more readily with the chelator. Examples of such reagents are known in the art and include dicyclohexylcarbodiimide (DCC), diethylazodicarboxylate (DEAD), and diisopropylazodicarboxylate (DIAD).

According to the method of the present invention, a quenching agent may optionally be added to the reaction solution after the Neutrokine-alpha conjugate is prepared to a sufficient degree. Suitable quenching agents are known in the art. In one embodiment, the quenching agent is glycine. In a specific embodiment, a solution comprising a glycine buffer, e.g., glycine HCl buffer, is used as a quenching agent. By way of example, after the reaction solution comprising the Neutrokine-alpha protein and the chelator has been mixed, agitated, or allowed to stand for a sufficient amount of time, e.g., 1-10 hours, 3-5 hours, or 1-3 hours, a solution comprising a glycine buffer is added to the reaction solution to stop the reaction. The reaction solution is then allowed to stand for an additional amount of time, e.g., about 0.25, 0.5, 0.75, 1, or 1.5 hours.

In a specific embodiment, the method of preparing a Neutrokine-alpha conjugate comprises mixing, agitating, or preparing a solution comprising:

a Neutrokine-alpha protein, wherein said protein comprises, or alternatively consists of a trimer of Neutrokine-alpha proteins, each consisting of amino acids 134-285 of SEQ ID NO: 2 and has a concentration of about 1 to about 2 mg/mL;

a MeO-DOTA-NCS in an amount such that the ratio MeO-DOTA-NCS molecules to lysine residues in a Neutrokine-alpha protein is about 12 to 1;

a citrate buffer having a concentration of about 0.5 to about 20 mM;

NaCl having a concentration of about 1 to about 200 mM;

HEPES buffer having a concentration of about 10 to about 500 mM; and

sterile water.

In an additional embodiment, the method of the present invention comprises:

preparing a first solution comprising a Neutrokine-alpha protein and a citrate buffer;

adding a second solution comprising a HEPES buffer to said first solution;

adding a third solution comprising a chelator, for example MeO-DOTA-NCS, and NaOH to said first solution;

optionally adjusting the pH of said first solution to about 8.5;

mixing, agitating, or allowing to stand said first solution at about 25° C. for about 3 to about 5 hours; and

optionally, adding quenching agent, e.g., a glycine buffer, to said first solution.

In a further embodiment, the mixing, agitating, or allowing to stand step is performed for about 4.5 to about 5.5 hours. In a further embodiment, the reaction solution can be incubated for an additional period of time after addition of the quenching agent.

In a further embodiment, the process of the invention is used to prepare a conjugate according to Formula IV.

The method of preparing a Neutrokine-alpha conjugate according to the present invention further optionally comprises a process of purifying said conjugate. A number of known methods may be used to purifying said protein conjugate, including but not limited to chromatography.

In one embodiment, the purifying step uses normal flow filtration or tangential flow filtration. In another embodiment, the process for purifying the conjugate according to the present invention is a diafiltration method (see, for example, Example 5).

In one embodiment, the Neutrokine-alpha conjugate is purified by HPLC, e.g., reverse phase HPLC.

Method of Preparing Complex

An additional embodiment of the present invention is directed to a method of preparing a Neutrokine-alpha complex. In one embodiment, a Neutrokine-alpha complex may be prepared by reacting a Neutrokine-alpha conjugate, as described herein, with a metal ion, such as a radionuclide, which is able to associate noncovalently with the conjugate. In another embodiment, the metal ion associates noncovalently with the chelator moiety of said Neutrokine-alpha conjugate. The reaction between the Neutrokine-alpha complex and the metal ion may occur in solution, such as in a suitable buffered solution.

In one embodiment, a method for preparing a Neutrokine-alpha complex comprises reacting a Neutrokine-alpha conjugate with a radionuclide. Such a method comprises mixing, agitating, or preparing a solution comprising a Neutrokine-alpha conjugate and a radionuclide. In one embodiment, said solution further comprises a buffer.

In another embodiment, the Neutrokine-alpha conjugate used in the present method is any specific Neutrokine-alpha conjugate as described above. In a particular embodiment, the Neutrokine-alpha conjugate comprises a Neutrokine-alpha protein comprising, or alternatively consisting of, amino acids 134-285 of SEQ ID NO:2 and chelator formed from MeO-DOTA-NCS.

In another embodiment, a method for preparing a Neutrokine-alpha complex comprises reacting a Neutrokine-alpha protein with a chelator complexed with a metal ion. In this embodiment, a chelator is first complexed with a metal ion according to known procedures in the art. For example, see U.S. Pat. No. 5,654,361. The chelator can be an activated chelator as described above. After the chelator is complexed with the metal ion and thereby forming a chelator-metal ion complex, the Neutrokine-alpha protein is reacted with the chelator-metal ion complex, using a procedure as described above for preparing a Neutrokine-alpha conjugate or using another procedure known in the art. According to the method, the solution comprising the Neutrokine-alpha protein and the chelator-metal ion complex is mixed, agitated, or prepared, thereby forming said Neutrokine-alpha complex.

When preparing a Neutrokine-alpha complex, the solution can be mixed, agitated, or allowed to stand for a variable number of hours, depending upon a number of variables, such factors including the identity of the chelator, the identity of the Neutrokine-alpha protein, the identity of the metal ion, the temperature of the reaction, the pH, the identity of the buffer, the molar ratio of the reactants, the purity of the available reagents, the presence of a catalyst, or activator, and other factors which would be evident to one of skill in the art. The solution comprising a Neutrokine-alpha conjugate and a metal ion can be mixed, agitated, or allowed to stand for any amount of time that permits sufficient formation of the Neutrokine-alpha complex. In one embodiment of the above process, the solution is mixed, agitated, or allowed to stand for about 0.5 minutes to about 24 hours. In another embodiment, the solution is mixed, agitated, or allowed to stand for about 1 hour to about 20 hours. In another embodiment, the solution is mixed, agitated, or allowed to stand for about 2 hours to about 10 hours. In another embodiment, the solution is mixed, agitated, or allowed to stand for about 1 minute to about 1 hour. In another embodiment, the solution is mixed, agitated, or allowed to stand for about 1 minute to about 30 minutes. In another embodiment, the solution is mixed, agitated, or allowed to stand for about 1, 2, 3, 4, 5, 6, 7, 8, 9, 10, 15, 20, 25, 30, 35, 40, 45, 50, or 55 minutes.

The pH of the solution used to prepare the Neutrokine-alpha complex of the invention can vary. In one embodiment, the pH is about 1, 2, 3, 4, 5, 6, 7, 8, 9, or 10. In another embodiment, said solution has a pH of about 4 to about 8. In another embodiment, said solution has a pH of about 5 to about 7. In another embodiment, said solution has a pH of about 5.5 to about 7. In another embodiment, said solution has a pH of about 6.5.

The solution used to prepare the Neutrokine-alpha complex may further comprise a buffer. Buffers that may be used according to the present invention are well-known in the art. Suitable buffers for use in the preparation of a Neutrokine-alpha complex include, but are not limited to, citrate, acetate, phosphate, carbonate, diphosphate, glycyl-glycine-piperazine-2HCl—NaOH; MES-NaOH—NaCl; TRIS-malic acid-NaOH; MES-NaOH; ACES-NaOH—NaCl; BES—NaOH—NaCl; MOPS—NaOH—NaCl; TES—NaOH—NaCl; MOPS—KOH; HEPES-NaOH—NaCl; TRIS—HCl; HEPPSO—NaOH; TAPS—NaOH—NaCl; HEPPS (EPPS)-NaOH; citric acid-disodiumhydrogenphosphate; boric acid-citric acid-potassium dihydrogen phosphate-Diethyl-barbituric acid-NaOH; citric acid-sodium citrate; sodium acetate-acetic acid; potassium hydrogenphthalate-NaOH; cacodylic acid sodium salt-HCl; potassium dihydrogen phosphate-disodium hydrogenphosphate; potassium dihydrogen-phosphate-NaOH; sodium dihydrogen phosphate-disodium hydrogen phosphate; imidazole-HCl; sodium tetraborate-boric acid; 2-amino-2-methyl-1,3-propanediol-HCl; diethanolamine-HCl; potassium chloride-boric acid-NaOH; boric acid-NaOH-KCl; glycine-NaOH; sodium bicarbonate, and sodium carbonate-sodium hydrogen carbonate.

In specific embodiments, the solution used to prepare the Neutrokine-alpha complex may comprise sodium acetate and sodium bicarbonate. In specific embodiments, the solution used to prepare the Neutrokine-alpha complex may comprise sodium acetate in a concentration range of about 50 mM to about 300 mM, preferably 200-250 mM, and sodium bicarbonate in a concentration range of about 50 mM to about 300 mM, preferably 50-100 mM. In specific embodiments, the solution used to prepare the Neutrokine-alpha complex may comprise about 220 mM sodium acetate and about 75 mM sodium bicarbonate. In specific embodiments, the solution used to prepare the Neutrokine-alpha complex may comprise 275 mM sodium acetate and 377 mM sodium bicarbonate in a ratio of about 1 to 10 to about 10 to 1. In specific embodiments, the solution used to prepare the Neutrokine-alpha complex may further comprise 275 mM sodium acetate and 377 mM sodium bicarbonate in a ratio of about 4 to 1.

The present invention encompasses varying the molar ratio of metal ions to chelator moieties on the Neutrokine-alpha conjugate in the preparation of a Neutrokine-alpha complex wherein a Neutrokine-alpha conjugate is reacted with a metal ion. In one embodiment, the ratio of metal ions to chelator moieties in Neutrokine-alpha conjugate is less than or equal to 1:100. In another embodiment, the ratio of metal ions to chelator moieties in Neutrokine-alpha conjugate is less than or equal to 1:80. In another embodiment, the ratio of metal ions to chelator moieties in Neutrokine-alpha conjugate is less than or equal to 1:50. In a preferred embodiment, the ratio of metal ions to chelator moieties in Neutrokine-alpha conjugate is exactly or approximately 1:30, 1:25, 1:20. 1:15: 1:10, 1:5 or 1:1. In a specific preferred embodiment, the ratio of metal ions to chelator moieties in Neutrokine-alpha conjugate is exactly or approximately 1:20.

In certain embodiments, preparation of a Neutrokine-alpha complex wherein a Neutrokine-alpha protein is reacted with a chelator-metal ion complex, the molar ratio of chelator-metal ion complex to chelator binding sites in Neutrokine-alpha protein is less than or equal to 1000:1. In other specific embodiments, molar ratio of chelator-metal ion complex to chelator binding sites in Neutrokine-alpha protein is less than or equal to 100:1. In preferred embodiments, molar ratio of chelator-metal ion complex to chelator binding sites in Neutrokine-alpha protein is 20:1, 15:1, 12:1, 11:1, 10:1 or 5:1.

Radionuclides which can be used in the present invention are known in the art, many of which are described and listed above and are available commercially. Additionally, several known methods can be used to prepare the radionuclides for use in the present invention. In one embodiment, the metal ion is in the form of a salt, for example a chloride salt. Such salts are known in the art. In another embodiment, the metal ion salt is yttrium chloride or yttrium acetate.

When preparing the complex of the present invention according to one embodiment of the invention, other metal ions which could compete for complexation with the chelator are not present in significant amounts in the solution. For example, when preparing a Neutrokine-alpha complex comprising ⁹⁰Y, Fe³⁺ is not present in the solution in a significant amount or concentration. As used with reference to the one or more competing metal ions in the present process, the phrase “significant amount or concentration” refers to an amount or concentration which significantly interferes, retards, delays, inhibits, or prevents preparation of the Neutrokine-alpha complex.

The method of preparing a Neutrokine-alpha complex may further comprise a step of removing excess metal ion from the reaction solution and/or the Neutrokine-alpha complex. In one embodiment, such a step comprises adding a secondary chelating agent which is able to complex with excess metal ion in the solution. Such a secondary chelating agent may include one or more known chelating agents, for example, DTPA, EDTA, and MeO-DOTA-glycine. In one embodiment, MeO-DOTA-glycine is used as a secondary chelating agent. In another embodiment, DTPA is used as a secondary chelating agent.

The method of preparing a Neutrokine-alpha complex may further comprise a step of removing excess metal ion from the reaction solution comprising the chelator-metal ion complex. In one embodiment, after the chelator-metal ion complex is formed and is in solution, a secondary chelating agent which is able to complex with excess metal ion is added. The solution is then eluted through a DEAE-cellulose anion exchange resin (Sigma Chemical Co., St. Louis, Mo.), which has been converted to acetate form to purify the neutral species chelator-metal ion complex from the charged species, i.e., secondary chelator-metal ion complex. The purified chelator-metal ion complex is then used to prepare the Neutrokine-alpha complex as described herein.

The secondary chelating agent can be added to the reaction mixture as a solid or as a solution. In one embodiment, the secondary chelating agent is added in a buffered solution. In another embodiment of the present invention, a buffer comprising an acetate buffer having a concentration of about 1 to about 50 mM, preferably about 10 mM, having a NaCl concentration of about 1 to about 500 mM, preferably about 140 mM, having a HSA concentration of about 1% to about 20%, preferably about 7% to about 8%, more preferably about 7.5%, having a pH of about 3-8, preferably about 6, and having a DTPA concentration of about 0.01 mM to about 100 mM, preferably about 1 mM, is added to the reaction solution after the Neutrokine-alpha complex is formed to a satisfactory level of completion.

In one embodiment, a solution having a NaCl concentration of about 1 to about 500 mM, preferably about 140 mM, having a pH of about 3-8, preferably about 6, and having a DTPA concentration of about 0.01 mM to about 100 mM, preferably about 2 mM, and comprising from about 0% to 20% sodium ascorbate, preferably 7-10% sodium ascorbate, is added to the reaction solution after the Neutrokine-alpha complex is formed to a satisfactory level of completion.

In one embodiment, the secondary chelating agent is added about 5 minutes after the formation of the Neutrokine-alpha complex. In other embodiments, the secondary chelating agent is added about 1, 2, 3, 4, 5, 6, 7, 8, 9, 10, 15, 20, 25, 30, 35, 40, 45, 50, 55, or 60 minutes after formation of the Neutrokine-alpha complex. In other embodiments, the secondary chelating agent is added at least 1, 2, 3, 4, 5, 6, 7, 8, 9, 10, 15, 20, 25, 30, 35, 40, 45, 50, 55, or 60 minutes after formation of the Neutrokine-alpha complex.

In another embodiment, the step of removing excess metal ion from the reaction solution and/or the Neutrokine-alpha complex comprises subjecting the reaction solution and/or the Neutrokine-alpha complex to centrifugation. Subjecting the reaction solution and/or the Neutrokine-alpha complex to centrifugation can remove non-chelated metal ions.

In another embodiment, the step of removing excess metal ion from the reaction solution and/or the Neutrokine-alpha complex comprises washing the excess metal ion from the reaction solution and/or the Neutrokine-alpha complex with a buffered solution. The washing may be repeated one or more times, as is necessary. In one embodiment, the washing is repeated two or three times. In one embodiment, the washing is repeated at least two or three times.

In one embodiment of the present method, two or more methods of removing excess metal ion are used in combination to remove excess metal ion from the step of removing excess metal ion from the reaction solution and/or the Neutrokine-alpha complex.

In another embodiment of the present method, for radiopharmaceutical and radiotherapy applications, the Neutrokine-alpha complex is prepared from a metal in an oxidation state different from that of the desired complex. In this case, either a reducing agent or an oxidizing agent, depending on the oxidation state of the metal used and the oxidation state of the desired final product, is added to the reaction mixture to bring the metal to the desired oxidation state. The oxidant or reductant can be used to form an intermediate complex in the desired oxidation state but with labile ligands. These labile ligands can then be displaced by the desired chelating moiety of the present invention. In another embodiment, the labile ligands are added to the reaction mixture along with the reductant or oxidant and the desired ligand to achieve the change to the desired oxidation state and chelation to the desired metal in a single step.

In a specific embodiment of the present invention, the method of preparing a Neutrokine-alpha complex comprises mixing, agitating, or preparing a solution comprising a Neutrokine-alpha conjugate according to Formula IV and metal ion selected from the group consisting of ⁹⁰Y, ¹¹¹In, ¹⁷⁷Lu, ¹⁶⁶Ho, ²¹⁵Bi, and ²²⁵Ac, wherein said Neutrokine-alpha protein comprises, or alternatively consists of, amino acids 134-285 of the sequence shown in Table 1 (SEQ ID NO:2).

In an additional embodiment, the method of preparing a Neutrokine-alpha complex comprises mixing, agitating, or preparing a solution comprising ¹¹¹In and a conjugate according to Formula IV, wherein said Neutrokine-alpha protein comprises, or alternatively consists of, amino acids 134-285 of the sequence shown in Table 1 (SEQ ID NO:2).

In an additional embodiment, the method of preparing a Neutrokine-alpha complex comprises mixing, agitating, or preparing a solution comprising ¹⁷⁷Lu and a conjugate according to Formula IV, wherein said Neutrokine-alpha protein comprises, or alternatively consists of, amino acids 134-285 of the sequence shown in Table 1 (SEQ ID NO:2).

In an additional embodiment, the method of preparing a Neutrokine-alpha complex comprises mixing, agitating, or preparing a solution comprising ⁹⁰Y and a conjugate according to Formula IV, wherein said Neutrokine-alpha protein comprises, or alternatively consists of, amino acids 134-285 of the sequence shown in Table 1 (SEQ ID NO:2).

In an additional embodiment, the method of preparing a Neutrokine-alpha complex comprises mixing, agitating, or preparing a solution comprising ²¹⁵Bi and a conjugate according to Formula IV, wherein said Neutrokine-alpha protein comprises, or alternatively consists of, amino acids 134-285 of the sequence shown in Table 1 (SEQ ID NO:2).

In an additional embodiment, the method of preparing a Neutrokine-alpha complex comprises mixing, agitating, or preparing a solution comprising ¹⁶⁶Ho and a conjugate according to Formula IV, wherein said Neutrokine-alpha protein comprises, or alternatively consists of, amino acids 134-285 of the sequence shown in Table 1 (SEQ ID NO:2).

In a further embodiment of the present invention, the method of preparing a Neutrokine-alpha complex comprises:

preparing a first solution comprising a Neutrokine-alpha conjugate;

adding to said first solution a second solution comprising a metal ion capable of complexing with said Neutrokine-alpha conjugate;

mixing, agitating, or allowing to stand said first solution; and

optionally, adding a secondary chelating agent, such as DTPA, to complex with uncomplexed metal ion.

In an additional embodiment, the method of preparing a Neutrokine-alpha complex comprises:

combining a first solution, second solution, and a third solution, wherein

said first solution comprises a Neutrokine-alpha conjugate,

said second solution comprises an acetate buffer, and

said third solution comprises ⁹⁰YCl₃;

mixing, agitating, or allowing to stand the combined solutions; and

adding to the combined solution a fourth solution, said fourth solution comprising MeO-DOTA-NCS, human serum albumin (HSA), acetate buffer, and NaCl.

Compositions

A further embodiment of the present invention is a composition comprising the conjugate or complex as described above. In one embodiment, a composition according to the present invention comprises a Neutrokine-alpha conjugate and an acceptable carrier, preferably a pharmaceutically acceptable carrier. Suitable acceptable carriers include any liquid or solvent in which the conjugate or complex can be dissolved or suspended. Suitable pharmaceutically acceptable carriers are well-known in the art. A composition comprising a Neutrokine-alpha conjugate may further comprise a metal ion, preferably a radionuclide.

In an additional embodiment, a composition according to the present invention comprises a Neutrokine-alpha complex and an acceptable carrier, preferably a pharmaceutically acceptable carrier. Suitable acceptable carriers include any liquid or solvent in which the conjugate or complex can be dissolved or suspended. Suitable pharmaceutically acceptable carriers are known in the art.

In an additional embodiment, a composition according to the present invention comprises a Neutrokine-alpha conjugate, a metal ion, and an acceptable carrier, preferably a pharmaceutically acceptable carrier. Suitable acceptable carriers include any liquid or solvent in which the conjugate or complex can be dissolved or suspended. Suitable pharmaceutically acceptable carriers are known in the art.

The concentration of the Neutrokine-alpha conjugate or Neutrokine-alpha complex in the composition of the present invention can vary. For example, the concentration of Neutrokine-alpha conjugate or complex can be from about 0.1 μg/mL to about 100 mg/mL, preferably from about 0.1 mg/mL to about 10 mg/mL, more preferably less than 4 mg/mL. In specific embodiments, the concentration of Neutrokine-alpha conjugate or Neutrokine-alpha complex in the composition is exactly or approximately 0.2 mg/mL. In specific embodiments, the concentration of Neutrokine-alpha conjugate or Neutrokine-alpha complex in the composition is exactly or approximately 2.0 mg/mL.

For compositions containing a radionuclide, either all or some chelator moieties may contain a metal ion. The amount of radionuclide incorporated into a Neutrokine-alpha complex or chelator-metal ion complex may be calculated as described in Example 6 and compared to expected values. For example, it may be desirable to design the Neutrokine-alpha conjugate of the invention such that it comprises an excess number of chelator moieties compared to the number of metal ions that are desired to be incorporated into the final Neutrokine-alpha complex. The advantage of this approach would be that the excess number of chelators would ensure that all metal ions are complexed into Neutrokine-alpha complexes thereby eliminating the need for steps to remove excess metal ions in the preparation of Neutrokine-alpha complexes. In a specific embodiment it is contemplated that approximately every chelator moiety in a solution of Neutrokine-alpha chelator becomes complexed with a metal ion. In another specific embodiment, it is contemplated that approximately 1 out of every 3 chelator moieties in a solution of Neutrokine-alpha chelator becomes complexed with a metal ion. In another specific embodiment, it is contemplated that approximately 1 out of every 5 chelator moieties in a solution of Neutrokine-alpha chelator becomes complexed with a metal ion. In another specific embodiment, it is contemplated that approximately 1 out of every 10 chelator moieties in a solution of Neutrokine-alpha chelator becomes complexed with a metal ion. In a specific and preferred embodiment, it is contemplated that approximately 1 out of every 20 chelator moieties in a solution of Neutrokine-alpha chelator becomes complexed with a metal ion. In a specific embodiment, it is contemplated that approximately 1 out of every 50 chelator moieties in a solution of Neutrokine-alpha chelator becomes complexed with a metal ion. In a specific embodiment, it is contemplated that approximately 1 out of every 100 chelator moieties in a solution of Neutrokine-alpha chelator becomes complexed with a metal ion.

In another specific embodiment, it is contemplated that at least 1 out of every 3 chelator moieties in a solution of Neutrokine-alpha chelator becomes complexed with a metal ion. In another specific embodiment, it is contemplated that at least 1 out of every 5 chelator moieties in a solution of Neutrokine-alpha chelator becomes complexed with a metal ion. In another specific embodiment, it is contemplated that at least 1 out of every 10 chelator moieties in a solution of Neutrokine-alpha chelator becomes complexed with a metal ion. In a specific and preferred embodiment, it is contemplated that at least 1 out of every 20 chelator moieties in a solution of Neutrokine-alpha chelator becomes complexed with a metal ion. In a specific embodiment, it is contemplated that at least 1 out of every 50 chelator moieties in a solution of Neutrokine-alpha chelator becomes complexed with a metal ion. In a specific embodiment, it is contemplated that at least 1 out of every 100 chelator moieties in a solution of Neutrokine-alpha chelator becomes complexed with a metal ion.

The conjugates of this invention, and in some instances the complexes of this invention, may be employed as a formulation. The formulation comprises a Neutrokine-alpha conjugate or Neutrokine-alpha complex and a physiologically acceptable carrier, excipient, or vehicle therefore. Thus, the formulation may consist of a physiologically acceptable carrier with a Neutrokine-alpha complex (Neutrokine-alpha protein+chelator moiety(ies)+metal ion), Neutrokine-alpha conjugate (Neutrokine-alpha protein+chelator moiety(ies)) or Neutrokine-alpha protein. The methods for preparing such formulations are well known. The formulation may be in the form of a suspension, injectable solution, or other suitable formulation. Physiologically acceptable suspending media, with or without adjuvants, may be used.

A composition described herein optionally further comprises one or more known stabilizers. The use of a stabilizer is particularly useful in compositions comprising a radionuclide. As is known in the art, one concern when administering any radiopharmaceutical is the potential for radiolytic degradation of the organic molecule present in the formulation, which may alter the biodistribution of the radioisotope or result in toxic by-products. Neither of these events is desirable. When high amounts of radioactivity are needed, there is the increased potential for radiation damage to the organic molecule. This degradation is more likely to occur when therapeutic radionuclides are used which are designed to deliver high radiation doses.

Examples of stabilizers that are suitable for use in the present compositions and methods include free radical inhibitors, such as benzyl chloride and ascorbic acid. See, e.g., H. Ikebuchi et al., Radioisotopes 26:451-457 (1977); B. J. Floor et al., J. Pharm. Sci. 74:197-200 (1985); A. Rego, et al., J. Pharm. Sci. 71:1219-23 (1982); and U.S. Pat. Nos. 5,843,396 and 5,384,113.

An additional embodiment of the present invention is a pharmaceutical composition comprising a Neutrokine-alpha conjugate and one or more pharmaceutically acceptable carriers. A preferred composition of the present invention is a pharmaceutical composition comprising a Neutrokine-alpha conjugate selected from a preferred group of Neutrokine-alpha conjugates as defined above, and one or more pharmaceutically acceptable excipients. A pharmaceutical composition that comprises Neutrokine-alpha conjugate may be formulated, as is well known in the art.

An additional embodiment of the present invention is a pharmaceutical composition comprising a Neutrokine-alpha complex and one or more pharmaceutically acceptable carriers. A preferred composition of the present invention is a pharmaceutical composition comprising a Neutrokine-alpha complex selected from a preferred group of Neutrokine-alpha complexes as defined above, and one or more pharmaceutically acceptable excipients. A pharmaceutical composition that comprises Neutrokine-alpha complex may be formulated, as is well known in the art.

In specific embodiments, a Neutrokine-alpha conjugate or Neutrokine-alpha complex is formulated in a solution comprising 10 mM sodium acetate and 140 mM sodium chloride, pH 6.0. In other specific embodiments, a Neutrokine-alpha conjugate or Neutrokine-alpha complex is formulated in a solution comprising 10 mM sodium acetate and 140 mM sodium chloride, 3-5% ascorbate, pH 6.0. Optionally, the formulation may also comprise 1 mM of the chelator diethylene triamine-N,N,N′,N″,N″-pentaacetic acid (DTPA).

The pharmaceutical composition of the invention can be administered to any animal that can experience the beneficial effects of the Neutrokine-alpha conjugate or the Neutrokine alpha complex of the invention. Foremost among such animals are humans, although the invention is not intended to be so limited.

The pharmaceutical composition of the present invention can be administered by any means that achieves its intended purpose. For example, administration can be by subcutaneous, intravenous, intramuscular, intraperitoneal, buccal, or ocular routes, rectally, parenterally, intrasystemically, intravaginally, topically (as by powders, ointments, drops or transdermal patch), or as an oral or nasal spray. The dosage administered will be dependent upon the age, health, and weight of the recipient, kind of concurrent treatment, if any, frequency of treatment, and the nature of the effect desired.

In addition to the Neutrokine-alpha conjugate or Neutrokine-alpha complex, the new pharmaceutical preparation can contain one or more suitable pharmaceutically acceptable carriers comprising excipients and auxiliaries that facilitate processing of the active compounds into preparations that can be used pharmaceutically.

The pharmaceutical preparation of the present invention is manufactured in a manner that is, itself, known, for example, by means of conventional mixing, granulating, dragee-making, dissolving, or lyophilizing processes. Thus, a pharmaceutical preparation for oral use can be obtained by combining the Neutrokine-alpha complex or the Neutrokine-alpha conjugate with one or more solid excipients, optionally grinding the resulting mixture and processing the mixture of granules, after adding suitable auxiliaries, if desired or necessary, to obtain tablets or dragee cores.

Pharmaceutical excipients are well known in the art. See, e.g., Remington's Pharmaceutical Sciences, Mack Publishing Co., Easton, Pa., USA. Suitable excipients include fillers such as saccharides, for example, lactose or sucrose, mannitol or sorbitol; cellulose preparations, and calcium phosphates, for example, tricalcium phosphate or calcium hydrogen phosphate; as well as binders, such as, starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer, such as, glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids, such as, fatty oils or liquid paraffin. In addition, stabilizers may be added.

A Neutrokine-alpha conjugate, Neutrokine-alpha complex, or Neutrokine-alpha composition according to the present invention may also be administered parenterally as an injectable dosage form in a physiologically acceptable diluent such as sterile liquids or mixtures thereof, including water, saline, aqueous dextrose and other pharmaceutically acceptable sugar solutions, alcohols such as ethanol, isopropanol, or hexadecyl alcohol, glycols such as propylene glycol and polyethylene glycol, glycerol ketals such as 2,2-dimethyl-1,3-dioxolane-4-methanol, ethers such as poly(ethyleneglycol)400, a pharmaceutically acceptable oil, fatty acid, fatty acid ester or glyceride, or an acetylated fatty acid glyceride with or without the addition of a pharmaceutically acceptable surfactant, such as a soap or detergent, suspending agent such as pectin, carbomers, methylcellulose, hydroxypropylmethylcellulose, or carboxymethylcellulose, an emulsifying agent or pharmaceutical adjuvants. In all cases, the form must be sterile and must be fluid to the extent that easy syringability exists. It must be stable under the conditions of manufacture and storage. It is also preferable that the composition is preserved against the contaminating action of micro-organisms such as bacteria and fungi.

Additional, suitable formulations for parenteral administration include aqueous solutions of the active compounds in water-soluble form, for example, water-soluble salts, alkaline solutions, and cyclodextrin inclusion complexes. Especially preferred alkaline salts are ammonium salts prepared, for example, with Tris, choline hydroxide, Bis-Tris propane, N-methylglucamine, or arginine.

In an additional embodiment, the composition is formulated in accordance with routine procedures as a pharmaceutical composition adapted for intravenous administration to human beings. Typically, compositions for intravenous administration are solutions in sterile, isotonic, aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutically acceptable oils which are useful in the formulation herein include, for example, those of petroleum, animal, vegetable, or synthetic origin, including peanut oil, soybean oil, sesame oil, cottonseed oil, olive oil, sunflower oil, petrolatum, and mineral oil. Fatty acids which may be used include, for example, oleic acid, stearic acid, and isostearic acid, while the fatty acid esters useful herein include ethyl oleate and isopropyl myristate. Suitable soaps include, for example, fatty acid alkali metal, ammonium, and triethanolamine salts. Acceptable detergents include cationic detergents and anionic detergents. Suitable cationic detergents include, for example, dimethyl dialkyl ammonium halides, alkyl pyridinium halides, and alkylamine acetates. Suitable anionic detergents include, for example, alkyl, aryl, and olefin sulfonates, alkyl, olefin, ether and monoglyceride sulfates, and sulfosuccinates. Useful non-ionic detergents include fatty amine oxides, fatty acid alkanolamides, and polyoxyethylenepolypropylene copolymers. Amphoteric detergents include alkyl-beta-aminopropionates and 2-alkylimidazoline quaternary salts, and mixtures thereof.

The parenteral compositions of this invention preferably will contain from about 0.1 mg/mL to about 10 mg/mL of the Neutrokine-alpha conjugate or Neutrokine-alpha complex as described herein in solution. In specific embodiments, the concentration of Neutrokine-alpha conjugate or Neutrokine-alpha complex in the parenteral composition is approximately 0.2 mg/mL. In specific embodiments, the concentration of Neutrokine-alpha conjugate or Neutrokine-alpha complex in the parenteral composition is approximately 2.0 mg/mL. The parenteral formulations in the form of sterile injectable solutions or suspensions will also preferably contain from about 0.05% to about 5% suspending agent in an isotonic medium. Buffers and preservatives may be added. A suitable surfactant may also be added. These surfactants may include polyethylene sorbitan fatty acid esters, such as sorbitan monooleate, and the high molecular weight adducts of ethylene oxide with a hydrophobic base, formed by the condensation of propylene oxide with propylene glycol.

In addition, suspensions of the active compounds as appropriate oily injection suspensions can be administered. Suitable lipophilic solvents or vehicles include, for example, fatty oils, for example, sesame oil, or synthetic fatty acid esters, for example, ethyl oleate or triglycerides or polyethylene glycol-400. Aqueous injection suspensions can contain substances that increase the viscosity of the suspension, for example, sodium carboxymethyl cellulose, sorbitol, and/or dextran. Optionally, the suspension may also contain stabilizers.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In nonpressurized powder compositions, the active ingredients in finely divided form may be used in admixture with a larger-sized pharmaceutically acceptable inert carrier comprising particles having a size, for example, of up to 100 micrometers in diameter. Suitable inert carriers include sugars such as lactose. Desirably, at least 95% by weight of the particles of the active ingredient have an effective particle size in the range of 0.01 to 10 micrometers.

Alternatively, the composition may be pressurized and contain a compressed gas, such as nitrogen or a liquefied gas propellant. The liquefied propellant medium and indeed the total composition are preferably such that the active ingredients do not dissolve therein to any substantial extent. The pressurized composition may also contain a surface-active agent. The surface-active agent may be a liquid or solid non-ionic surface-active agent or may be a solid anionic surface-active agent. It is preferred to use the solid anionic surface-active agent in the form of a sodium salt.

A further form of topical administration is to the eye. The compounds and compositions of the present invention are delivered in a pharmaceutically acceptable ophthalmic vehicle, such that the compounds are maintained in contact with the ocular surface for a sufficient time period to allow the compounds to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material.

Compositions for rectal or vaginal administration are preferably suppositories which can be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the drugs.

Another embodiment of the present invention is a composition comprising a Neutrokine-alpha conjugate or a Neutrokine-alpha complex and a diagnostically suitable carrier.

One or more lyophilization aids may be used in the preparation a Neutrokine-alpha conjugate, a Neutrokine-alpha complex, or compositions thereof. Such lyophilization aids include but are not limited to mannitol, lactose, sorbitol, dextran, Ficoll, and polyvinylpyrrolidine(PVP).

One or more stabilization aids may be used in the preparation a Neutrokine-alpha conjugate, a Neutrokine-alpha complex, or compositions thereof. Such stabilization aids include but are not limited to ascorbic acid, cysteine, monothioglycerol, sodium bisulfite, sodium metabisulfite, gentisic acid, and inositol.

One or more solubilization aids may be used in the preparation a Neutrokine-alpha conjugate, a Neutrokine-alpha complex, or compositions thereof. Such solubilization aids include but are not limited to ethanol, glycerin, polyethylene glycol, propylene glycol, polysorbates and lecithin.

One or more bacteriostats may be used in the preparation a composition comprising a Neutrokine-alpha conjugate or a Neutrokine-alpha complex. Such bacteriostats include but are not limited to benzyl alcohol, benzalkonium chloride, chlorbutanol, and methyl, propyl or butyl paraben.

In specific embodiment, the composition of the present invention is formulated using the BEMA™ BioErodible Mucoadhesive System, MCA™ MucoCutaneous Absorption System, SMP™ Solvent MicroParticle System, or BCP™ BioCompatible Polymer System of Atrix Laboratories, Inc. (Fort Collins, Colo.).

Sustained-release compositions also include liposomally entrapped compositions of the invention (see generally, Langer, Science 249:1527-1533 (1990); Treat et al., in “Liposomes in the Therapy of Infectious Disease and Cancer,” Lopez-Berestein and Fidler (eds.), Liss, New York, pp. 317-327 and 353-365 (1989)). Liposomes comprising a Neutrokine-alpha complex or conjugate may be prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

In another embodiment, a sustained release composition of the invention includes crystal formulations known in the art.

In yet an additional embodiment, a composition of the invention is delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

The composition comprising the Neutrokine-alpha conjugate or Neutrokine-alpha complex to be used for therapeutic administration must be sterile. Sterility is readily accomplished by filtration through sterile filtration membranes (e.g., 0.2 micron membranes). A therapeutic Neutrokine-alpha conjugate or Neutrokine-alpha complex compositions generally is placed into a container having a sterile access port, for example, an intravenous solution bag or vial having a stopper pierceable by a hypodermic injection needle.

Pharmaceutical compositions containing Neutrokine-alpha conjugate or Neutrokine-alpha complex of the invention may be administered orally, rectally, parenterally, subcutaneously, intracistemally, intravaginally, intraperitoneally, topically (as by powders, ointments, drops or transdermal patch), bucally, or as an oral or nasal spray (e.g., via inhalation of a vapor or powder). In one embodiment, “pharmaceutically acceptable carrier” means a non-toxic solid, semisolid or liquid filler, diluent, encapsulating material or formulation auxiliary of any type. In a specific embodiment, “pharmaceutically acceptable” means approved by a regulatory agency of the federal or a state government or listed in the U.S. Pharmacopeia or other generally recognized pharmacopeia for use in animals, and more particularly humans. Nonlimiting examples of suitable pharmaceutical carriers according to this embodiment are provided in “Remington's Pharmaceutical Sciences” by E. W. Martin, and include sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The term “parenteral” as used herein refers to modes of administration which include intravenous, intramuscular, intraperitoneal, intrasternal, subcutaneous and intraarticular injection and infusion.

In one embodiment, a composition comprising a Neutrokine-alpha conjugate or a Neutrokine-alpha complex is administered subcutaneously.

In another embodiment, a composition comprising a Neutrokine-alpha conjugate or a Neutrokine-alpha complex is administered intravenously.

A composition comprising a Neutrokine-alpha conjugate or a Neutrokine-alpha complex is also suitably administered by sustained-release systems. Suitable examples of sustained-release compositions include suitable polymeric materials (such as, for example, semi-permeable polymer matrices in the form of shaped articles, e.g., films, or mirocapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly-(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., id.) or poly-D-(−)-3-hydroxybutyric acid (EP 133,988).

In another embodiment, a composition comprising a Neutrokine-alpha conjugate or a Neutrokine-alpha complex is formulated in a biodegradable, polymeric drug delivery system, for example as described in U.S. Pat. Nos. 4,938,763; 5,278,201; 5,278,202; 5,324,519; 5,340,849; and 5,487,897 and in International Publication Numbers WO001/35929, WO00/24374, and WO00/06117 which are hereby incorporated by reference in their entirety. In specific preferred embodiments, the composition comprising a Neutrokine-alpha conjugate or Neutrokine-alpha complexes formulated using the ATRIGEL® Biodegradable System of Atrix Laboratories, Inc. (Fort Collins, Colo.). In other specific embodiments, a composition comprising a Neutrokine-alpha conjugate or a Neutrokine-alpha complex is formulated using the ProLease® sustained release system available from Alkermes, Inc. (Cambridge, Mass.).

Examples of biodegradable polymers which can be used in the formulation of a composition comprising a Neutrokine-alpha conjugate or a Neutrokine-alpha complex, include but are not limited to, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), polyvinylpyrrolidone, polyethylene glycol, polyhydroxycellulose, chitin, chitosan, and copolymers, terpolymers, or combinations or mixtures of the above materials. The preferred polymers are those that have a lower degree of crystallization and are more hydrophobic. These polymers and copolymers are more soluble in the biocompatible solvents than the highly crystalline polymers such as polyglycolide and chitin which also have a high degree of hydrogen-bonding. Preferred materials with the desired solubility parameters are the polylactides, polycaprolactones, and copolymers of these with glycolide in which there are more amorphous regions to enhance solubility. In specific preferred embodiments, the biodegradable polymers which can be used in the formulation of a composition comprising a Neutrokine-alpha conjugate or a Neutrokine-alpha complex are poly(lactide-co-glycolides). Polymer properties such as molecular weight, hydrophobicity, and lactide/glycolide ratio may be modified to obtain the desired Neutrokine-alpha conjugate or Neutrokine-alpha complex release profile (See, e.g., Ravivarapu et al., Journal of Pharmaceutical Sciences 89:732-741 (2000), which is hereby incorporated by reference in its entirety).

It is also preferred that the solvent for the biodegradable polymer be non-toxic, water miscible, and otherwise biocompatible. Examples of such solvents include, but are not limited to, N-methyl-2-pyrrolidone, 2-pyrrolidone, C₂ to C₆ alkanols, C₁ to C₁₅ alcohols, diols, triols, and tetraols such as ethanol, glycerine propylene glycol, butanol; C₃ to C₁₅ alkyl ketones such as acetone, diethyl ketone and methyl ethyl ketone; C₃ to C₁₅ esters such as methyl acetate, ethyl acetate, ethyl lactate; alkyl ketones such as methyl ethyl ketone, C₁ to C₁₅ amides such as dimethylformamide, dimethylacetamide and caprolactam; C₃ to C₂₀ ethers such as tetrahydrofuran, or solketal; tweens, triacetin, propylene carbonate, decylmethylsulfoxide, dimethyl sulfoxide, oleic acid, 1-dodecylazacycloheptan-2-one. Other preferred solvents are benzyl alchohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of the solvating ability and their compatibility.

Additionally, formulations comprising a Neutrokine-alpha conjugate or a Neutrokine-alpha complex and a biodegradable polymer may also include release-rate modification agents and/or pore-forming agents. Examples of release-rate modification agents include, but are not limited to, fatty acids, triglycerides, other like hydrophobic compounds, organic solvents, plasticizing compounds and hydrophilic compounds. Suitable release rate modification agents include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di-(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; sterols, such as cholesterol; alcohols, such as C6-C12 alkanols, 2-ethoxyethanol, and the like. The release rate modification agent may be used singly or in combination with other such agents. Suitable combinations of release rate modification agents include, but are not limited to, glycerin/propylene glycol, sorbitol/glycerine, ethylene oxide/propylene oxide, butylene glycol/adipic acid, and the like. Preferred release rate modification agents include, but are not limited to, dimethyl citrate, triethyl citrate, ethyl heptanoate, glycerin, and hexanediol. Suitable pore-forming agents that may be used in the polymer composition include, but are not limited to, sugars such as sucrose and dextrose, salts such as sodium chloride and sodium carbonate, polymers such as hydroxylpropylcellulose, carboxymethylcellulose, polyethylene glycol, and polyvinylpyrrolidone. Solid crystals that will provide a defined pore size, such as salt or sugar, are preferred.

For parenteral administration, in one embodiment, a Neutrokine-alpha conjugate or a Neutrokine-alpha complex is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting a Neutrokine-alpha conjugate or a Neutrokine-alpha complex uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, sucrose, or dextrins; chelating agents such as EDTA; sugar alcohols such as mannitol or sorbitol; counterions such as sodium; preservatives, such as cresol, phenol, chlorobutanol, benzyl alcohol and parabens, and/or nonionic surfactants such as polysorbates, poloxamers, or PEG.

In a specific embodiment, a composition of the invention comprises, about between 0.1 mg/mL and 20 mg/mL of a Neutrokine-alpha conjugate or a Neutrokine-alpha complex wherein the Neutrokine-alpha protein of said conjugate or complex comprises amino acid residues 134-285 of SEQ ID NO:2, 10.0 mM sodium citrate, 140.0 mM sodium chloride, 8.7 mM HEPES, 4% (w/v) sodium ascorbate, and 3.3% (w/v) Gentran-40. In a specific embodiment, a composition of the invention comprises, between 1 mg/mL and 10 mg/mL of a Neutrokine-alpha conjugate or a Neutrokine-alpha complex wherein the Neutrokine-alpha protein of said conjugate or complex comprises amino acid residues 134-285 of SEQ ID NO:2, 10.0 mM sodium citrate, 140.0 mM sodium chloride, 8.7 mM HEPES, 4% (w/v) sodium ascorbate, and 3.3% (w/v) Gentran-40. In a specific embodiment, a composition of the invention comprises, between 2 mg/mL and 8 mg/mL of a Neutrokine-alpha conjugate or a Neutrokine-alpha complex wherein the Neutrokine-alpha protein of said conjugate or complex comprises amino acid residues 134-285 of SEQ ID NO:2, 10.0 mM sodium citrate, 140.0 mM sodium chloride, 8.7 mM HEPES, 4% (w/v) sodium ascorbate, and 3.3% (w/v) Gentran-40. In a specific embodiment, a composition of the invention comprises, between 3 mg/mL and 6 mg/mL of a Neutrokine-alpha conjugate or a Neutrokine-alpha complex wherein the Neutrokine-alpha protein of said conjugate or complex comprises amino acid residues 134-285 of SEQ ID NO:2, 10.0 mM sodium citrate, 140.0 mM sodium chloride, 8.7 mM HEPES, 4% (w/v) sodium ascorbate, and 3.3% (w/v) Gentran-40. The above described compositions may be used as pharmaceutical compositions.

A composition of the invention may be administered alone or in combination with other therapeutic agents, including but not limited to, chemotherapeutic agents, antibiotics, antivirals, steroidal and non-steroidal anti-inflammatories, conventional immunotherapeutic agents, immunosuppresants, and cytokines. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the compositions of the invention are administered in combination with anti-TNF-alpha antibodies such as infliximab (also known as Remicade™, Centocor, Inc.).

In one embodiment, the compositions of the invention are administered in combination with ENBREL™ (Etanercept).

In one embodiment, the compositions of the invention are administered in combination with anti-CD20 antibodies such as Ibritumomab Tiuxetan (Zevalin™) or rituximab (Rituxan™).

In one embodiment, the compositions of the invention are administered in combination with anti-TRAIL-R1 and/or TRAIL-R2 antibodies, for example those described in WO02/97033 and WO02/79377, which are hereby incorporated by reference in their entireties.

In one embodiment, a composition of the invention is administered in combination with one or more other members of the TNF family. TNF, TNF-related, or TNF-like molecules that may be administered with the compositions of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), AIM-II (International Publication No. WO 97/34911), APRIL (J. Exp. Med. 188(6):1185-1190), endokine-alpha (International Publication No. WO 98/07880), TR6 (International Publication No. WO 98/30694), OPG, and Neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (International Publication No. WO 98/06842), and TR12.

In another embodiment, a composition of the invention is administered in combination with CD40 ligand (CD40L), a soluble form of CD40L (e.g., AVREND™), biologically active fragments, variants, or derivatives of CD40L, anti-CD40L antibodies (e.g., agonistic or antagonistic antibodies), and/or anti-CD40 antibodies (e.g., agonistic or antagonistic antibodies).

Demonstration of Therapeutic or Prophylactic Activity

The Neutrokine-alpha conjugate, Neutrokine-alpha complex, and compositions thereof of the invention are preferably tested in vitro, and then in vivo, for the desired therapeutic or prophylactic activity, prior to use in humans. For example, in vitro assays to demonstrate the therapeutic or prophylactic utility of a compound or pharmaceutical composition include the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated include in vitro cell culture assays in which a patient tissue sample is grown in culture and exposed to or otherwise administered a compound, and the effect of such compound upon the tissue sample is observed.

Furthermore, a number of cell lines can be used to test for the binding of a Neutrokine-alpha conjugate or Neutrokine-alpha complex of the invention. Suitable cell lines that may be used to test this binding include, for example: IM-9 (ATCC CCL-159); Reh (ATCC CRL-8286); ARH-77 (ATCC CRL-1621); Raji (ATCC CCL-86); Namalwa (CRL-1432); RPMI 8226 (ATCC CCL-155). Additionally, one could use other cell lines (e.g., CHO, NSO) transfected with expression constructs for receptors for Neutrokine-alpha. Suitable expression constructs include expression constructs which encode receptors for Neutrokine-alpha, e.g., BAFF-R (SEQ ID NO:5), TACI (SEQ ID NO:7), and/or BCMA (SEQ ID NO:9). A Neutrokine-alpha conjugate or Neutrokine-alpha complex of the invention will bind to a receptor capable of binding a Neutrokine-alpha protein. Cells exposed to Neutrokine-alpha complexes of the invention may further be assessed for viability.

Diagnostic Uses of Neutrokine-Alpha Conjugates and Neutrokine-Alpha Complexes

Neutrokine-alpha receptors are expressed primarily on B cells (see beginning of Therapeutic Uses of Neutrokine-Alpha Conjugates and Neutrokine-Alpha Complexes Section below). Herein, Neutrokine-alpha receptors refer not only to Neutrokine-alpha receptors such as BAFF-R (SEQ ID NO:5), TACI (SEQ ID NO:7), and/or BCMA (SEQ ID NO:9, but also to allelic variants, conserved variants, synthetic fragments and/or biologically processed fragments of Neutrokine-alpha receptors that are capable of binding Neutrokine-alpha.

Accordingly, in one embodiment, a Neutrokine-alpha conjugate or Neutrokine-alpha complex of the invention is used to quantitate or qualitate concentrations of B lineage cells expressing Neutrokine-alpha receptor on their cell surfaces (e.g., normal B cells as well as B cell related leukemias or lymphomas).

In one embodiment, Neutrokine-alpha conjugates and/or Neutrokine-alpha complexes of the invention may be used in diagnostic applications comprising detecting Neutrokine-alpha receptor expression (BAFF-R (SEQ ID NO:5), TACI (SEQ ID NO:7), and/or BCMA (SEQ ID NO:9)). Neutrokine-alpha conjugates and/or Neutrokine-alpha complexes of the invention may also be used to determine the structure and/or temporal, tissue, cellular, or subcellular location of Neutrokine-alpha receptors and/or to determine the activity of signalling pathways associated with Neutrokine-alpha. These diagnostic assays may be performed in vitro, such as, for example, on blood samples or biopsy tissue, or in vivo, using techniques described herein or otherwise known in the art.

In specific embodiments, Neutrokine-alpha conjugates and/or Neutrokine-alpha complexes of the invention may be used to diagnose cancers, particularly lymphocytic cancers. For example, Neutrokine-alpha conjugates and/or Neutrokine-alpha complexes of the invention may be used to detect cancer cells that express Neutrokine-alpha receptors. Diagnosis of cancer may be made on the basis of increased or decreased expression of Neutrokine-alpha receptors on cancer cells compared to non-cancerous Neutrokine-alpha receptor expressing cells (normal B cells). Alternatively, diagnosis of cancer may be made on the basis of combined expression of Neutrokine-alpha receptor expression, at normal or aberrant levels, in combination with expression of other markers that are diagnostic of cancer.

As a non-limiting example, Neutrokine-alpha conjugates and/or Neutrokine-alpha complexes of the invention, may be used to determine the presence or absence of Neutrokine-alpha receptors on cells from a patient that is a candidate for treatment with the Neutrokine-alpha conjugates and/or Neutrokine-alpha complexes of the invention. In specific embodiments, the patients are cancer patients, particularly cancer patients with lymphocytic cancers, such as multiple myeloma and Non-Hodgkin's lymphoma. Expression of Neutrokine-alpha receptors on cancer cells from such cancer patients would indicate that therapy with Neutrokine-alpha conjugates and/or Neutrokine-alpha complexes of the invention is likely to be effective, whereas the absence of detectable expression would suggest the patient probably would not benefit from therapy with Neutrokine-alpha conjugates and/or Neutrokine-alpha complexes of the invention.

As another non-limiting example, Neutrokine-alpha conjugates and/or Neutrokine-alpha complexes of the invention may be used to localize and/or quantitate Neutrokine-alpha receptor expressing cells, e.g., multiple myeloma cells, non-Hodgkin's lymphoma, or chronic lymphocytic leukemia cells.

In one embodiment, a Neutrokine-alpha conjugate or Neutrokine-alpha complex of the invention is used to diagnose or monitor an individual having an immunodeficiency. Examples of immunodeficiencies that may be diagnosed or monitored with a Neutrokine-alpha conjugate or Neutrokine-alpha complex of the invention, include, but are not limited to, common variable immunodeficiency (CVID) and diseases characterized by deficiencies in one or more classes or subclasses of immunoglobulin (e.g., selective IgA deficiency, ataxia telangiectsia, X-linked agammaglobulinemia, severe combined immunodeficiency (SCID), Wiskott-Aldrich syndrome, and hyper IgM syndrome)

Diagnosis of an immunodeficiency may be made on the basis of increased or decreased expression of Neutrokine-alpha receptors on known Neutrokine-alpha receptor expressing cells (e.g., lymphocytes, particularly B cells) compared to Neutrokine-alpha receptor expressing cells from patients without an immunodeficiency. Immunodeficiencies may be characterized by decreased levels of Neutrokine-alpha receptor expression, suggesting the immunodeficiency may be the result of decreased or absent Neutrokine-alpha receptor activity. Alternatively, increased levels of Neutrokine-alpha receptor expression may also be indicative of an immunodeficiency if, for example, Neutrokine-alpha receptors are only partially functional and therefore, more receptors are necessary to deliver Neutrokine-alpha receptor-mediated signaling. Thus, diagnosis of an immunodeficiency may be made on the basis of increased or decreased expression of Neutrokine-alpha receptors compared to Neutrokine-alpha receptor expression in a patient without the immunodeficiency. Alternatively, diagnosis of an immunodeficiency may be made on the basis of expression of Neutrokine-alpha receptor expression, at normal or aberrant levels, in combination with expression of other markers that are diagnostic of an immunodeficiency (e.g. cell surface or serum immunoglobulin expression).

In another embodiment, a Neutrokine-alpha conjugate or Neutrokine-alpha complex of the invention is used to diagnose or monitor an individual having an autoimmune disease or disorder, particularly an autoimmune disease associated with the production of autoantibodies. Examples of autoimmune diseases that may be diagnosed or monitored with a Neutrokine-alpha conjugate or Neutrokine-alpha complex of the invention, include, but are not limited to, rheumatoid arthritis, systemic lupus erythematosus (SLE), and Sjögren's syndrome.

Diagnosis of an autoimmune disease may be made on the basis of increased or decreased expression of Neutrokine-alpha receptors on known Neutrokine-alpha receptor expressing cells (e.g., lymphocytes, particularly B cells) compared to Neutrokine-alpha receptor expressing cells from patients without an autoimmune disease. Autoimmune diseases may be characterized by increased levels of Neutrokine-alpha receptor expression, suggesting the autoimmune disease may be the result of excess Neutrokine-alpha receptor activity. Alternatively, decreased levels of Neutrokine-alpha receptor expression may also be indicative of an autoimmune disease, if for example, Neutrokine-alpha receptors are more easily activated to signal or even are constitutively active in the absence of ligand, and therefore, fewer receptors are necessary to deliver Neutrokine-alpha receptor-mediated signaling. Thus, diagnosis of an autoimmune disease may be made on the basis of increased or decreased expression of Neutrokine-alpha receptors compared to Neutrokine-alpha receptor expression in a patient without the autoimmune disease. Alternatively, diagnosis of an autoimmune disease may be made on the basis of expression of Neutrokine-alpha receptor expression, at normal or aberrant levels, in combination with expression of other markers that are diagnostic of an autoimmune disease (e.g., cell surface or serum immunoglobulin expression).

Any means described herein or otherwise known in the art may be applied to detect Neutrokine-alpha receptors (e.g., FACS analysis or ELISA detection of Neutrokine-alpha proteins of the invention. Additionally, hybridization or PCR detection of Neutrokine-alpha receptor polynucleotides of the invention (BAFF-R (SEQ ID NO:5), TACI (SEQ ID NO:7), and/or BCMA (SEQ ID NO:9) and to determine the expression profile of Neutrokine-alpha polynucleotides and/or polypeptides of the invention in a biological sample.

By analyzing or determining the expression level of the gene encoding the Neutrokine-alpha receptor is intended qualitatively or quantitatively measuring or estimating the level of the Neutrokine-alpha receptor polypeptide in a first biological sample either directly (e.g., by determining or estimating absolute protein level) or relatively (e.g., by comparing to the Neutrokine-alpha receptor present in a second biological sample). In one embodiment, the Neutrokine-alpha receptor level in the first biological sample is measured or estimated and compared to a standard Neutrokine-alpha receptor level, the standard being taken from a second biological sample obtained from an individual not having the disorder or being determined by averaging levels from a population of individuals not having a disorder of the immune system. As will be appreciated in the art, once a standard Neutrokine-alpha receptor level is known, it can be used repeatedly as a standard for comparison.

By “biological sample” is intended any biological sample obtained from an individual, body fluid, cell line, tissue culture, or other source. As indicated, biological samples include body fluids (such as sera, plasma, urine, synovial fluid and spinal fluid), immune system tissue, and other tissue sources. Methods for obtaining tissue biopsies and body fluids from mammals are well known in the art.

The Neutrokine-alpha conjugate or complex of the present invention may, additionally, be employed histologically, as in immunofluorescence, immunoelectron microscopy, or non-immunological assays, for in situ detection of Neutrokine-alpha receptors. In situ detection may be accomplished by removing a histological specimen from a patient, and applying thereto a labeled Neutrokine-alpha conjugate or Neutrokine-alpha complex of the present invention. The Neutrokine-alpha conjugate or Neutrokine-alpha complex is preferably applied by overlaying the Neutrokine-alpha conjugate or Neutrokine-alpha complex onto a biological sample. Through the use of such a procedure, it is possible to determine not only the presence of Neutrokine-alpha receptors, but also their distribution in the examined tissue. Using the present invention, those of ordinary skill will readily perceive that any of a wide variety of histological methods (such as staining procedures) can be modified in order to achieve such in situ detection.

Immunoassays and non-immunoassays for Neutrokine-alpha receptor products or conserved variants or peptide fragments thereof will typically comprise incubating a sample, such as a biological fluid, a tissue extract, freshly harvested cells, or lysates of cells which have been incubated in cell culture, in the presence of Neutrokine-alpha conjugate or Neutrokine-alpha complex capable of identifying Neutrokine-alpha receptors, and detecting the bound Neutrokine-alpha conjugate or Neutrokine-alpha complex by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the Neutrokine-alpha conjugate or Neutrokine-alpha complex. The solid phase support may then be washed with the buffer a second time to remove unbound Neutrokine-alpha conjugate or Neutrokine-alpha complex. Optionally, the Neutrokine-alpha conjugate or Neutrokine-alpha complex is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot Neutrokine-alpha conjugate or Neutrokine-alpha complex may be determined according to well-known methods. Those skilled in the art will be able to determine operative and optimal assay conditions for each determination by employing routine experimentation. In particular, cell lines as described above may be used.

In addition to assaying Neutrokine-alpha receptor levels in a biological sample obtained from an individual, Neutrokine-alpha receptors can also be detected in vivo by imaging. For example, in one embodiment of the invention, a Neutrokine-alpha conjugate or Neutrokine-alpha complex is used to image B cell lymphomas. In another embodiment, Neutrokine-alpha conjugate or Neutrokine-alpha complex is used to image lymphomas (e.g., monocyte and B cell lymphomas).

Labels or markers for in vivo imaging of Neutrokine-alpha conjugate or Neutrokine-alpha complex include those detectable by X-radiography, NMR, MRI, CAT-scans or ESR. For X-radiography, suitable labels include radioisotopes such as barium or cesium, which emit detectable radiation but are not overtly harmful to the subject.

In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)).

Detection may also be accomplished using any of a variety of other immunoassays. For example, by radioactively labeling the Neutrokine-alpha conjugate or Neutrokine-alpha complex, it is possible to detect Neutrokine-alpha receptors through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by means including, but not limited to, a gamma counter, a scintillation counter, or autoradiography.

The Neutrokine-alpha conjugate or Neutrokine-alpha complex can also be detectably labeled using fluorescence emitting metals such as ¹⁵²Eu, or others of the lanthanide series.

Therapeutic Uses of Neutrokine-Alpha Conjugates and Neutrokine-Alpha Complexes

Neutrokine-alpha receptor expression, defined functionally by the binding of biotinylated Neutrokine-alpha to cells, is found predominantly on B cells (see, e.g., Moore et al., Science 285:260-263 (1999), which is hereby incorporated by reference in its entirety), although TACI has also been reported to be expressed on activated T cells (Wang et al., Nature Immunol. 2:577-8 (2001), which is hereby incorporated by reference in its entirety.) Furthermore, Neutrokine-alpha receptor expression is not observed in pre-B cells, rather Neutrokine-alpha receptor expression becomes observable at the same stage in B cell development when surface Ig expression becomes apparent (see, e.g., Hsu et al., J. Immunol. 168:5993-6 (2002), which is hereby incorporated by reference in its entirety).

Receptors which bind proteins comprising Neutrokine-alpha or fragments or variants thereof may also be expressed on non-hematopoietic cells. In specific embodiments, receptors which bind proteins comprising Neutrokine-alpha or fragments or variants thereof (e.g., Neutrokine-alpha heterotimers (described below) comprising one or two APRIL monomers) are expressed on cells or cell lines of fibroblastic or epithelial lineage or having fibroblastic or epithelial morphology (e.g., NIH-3T3 fibroblasts, A549 lung carcinoma cells or HT-29 colorectal adenocarcinoma cells).

Additionally, Neutrokine-alpha receptor expression, defined functionally by the binding of biotinylated Neutrokine-alpha to cells, has been observed on multiple myeloma, Non-Hodgkin's lymphoma, and chronic lymphocytic leukemia primary tumor explants (see, e.g., Briones et al., Experimental Hematology 30:135-141 (2002) and Novak et al., Blood 100:2973-2979 (2002), each of which is hereby incorporated by reference in its entirety) and on B lineage immortalized hematopoietic cell lines (e.g., IM-9 (ATCC CCL-159); Reh (ATCC CRL-8286); ARH-77 (ATCC CRL-1621); Raji (ATCC-CCL-86); Namalwa (CRL-1432); RPMI 8226 (ATCC CCL-155)).

The restricted expression profile of Neutrokine-alpha receptor expression described above makes Neutrokine-alpha an attractive vehicle for targeting therapies to lymphocytes, and to B lineage cells in particular.

Biodistribution studies of a Neutrokine-alpha complex injected into BALB/c mice illustrate that a Neutrokine-alpha conjugate or a Neutrokine-alpha complex has high in vivo targeting specificity for lymphoid tissues such as spleen and lymph nodes (See Example 1). Thus in a specific embodiment, the invention provides a method for the specific destruction or disablement of lymphoid tissue (e.g., lymph nodes and spleen) comprising administering a Neutrokine-alpha conjugate or a Neutrokine-alpha complex. In a preferred embodiment, the lymphoid tissue is not permanently destroyed but rather is temporarily disabled, (e.g., cells of hematopoietic lineage in lymphoid tissues are destroyed/killed while a Neutrokine-alpha conjugate or a Neutrokine-alpha complex is administered, but these populations recover once administration of the Neutrokine-alpha conjugate or the Neutrokine-alpha complex is stopped.)

The Neutrokine-alpha conjugate, Neutrokine-alpha complex, or composition of the present invention is useful as a therapeutic agent for the treatment of diseases and conditions associated with cells that express Neutrokine-alpha receptors. Because Neutrokine-alpha receptors are known to be expressed predominantly on B cells, it is a preferred embodiment of the present invention that Neutrokine-alpha conjugates or complexes are useful for treating diseases involving B cells or B cell activity.

TACI is also known to be expressed by T cells, specifically activated T cells. Therefore, in other embodiments, Neutrokine-alpha conjugates or complexes may also be useful for treating diseases involving T cells or T cell activity.

The treatment and/or prevention of diseases, disorders, or conditions associated with aberrant expression and/or activity of a Neutrokine-alpha protein and/or a receptor for the Neutrokine-alpha (e.g., BAFF-R, TACI, BCMA,) includes, but is not limited to, alleviating symptoms associated with those diseases, disorders or conditions. The conjugate or complex of the invention may also be used to target and kill cells which bind Neutrokine-alpha.

In particular, the Neutrokine-alpha conjugate or Neutrokine-alpha complex are administered as a form of radiotherapy. In one embodiment, the method of the present invention comprises administering to a subject a composition comprising a Neutrokine-alpha complex and a pharmaceutically acceptable carrier, for radiotherapeutic treatment of said subject wherein the Neutrokine-alpha complex is administered in an amount sufficient for the needed treatment. In another embodiment, the method further comprises monitoring said patient. In another embodiment, the subject is a mammal. In a further embodiment, the subject is a human.

In another embodiment, the method of the present invention comprises administering to a subject a composition comprising a Neutrokine-alpha conjugate and a pharmaceutically acceptable carrier, for therapeutic treatment of said subject wherein the Neutrokine-alpha conjugate is administered in an amount sufficient for the needed treatment. In another embodiment, the method further comprises monitoring said patient. In another embodiment, the subject is a mammal. In a further embodiment, the subject is a human.

In one embodiment, the invention provides a method of target compositions comprising a Neutrokine-alpha conjugate or a Neutrokine-alpha complex to Neutrokine-alpha receptor expressing cells, such as, for example, B cells or T cells. A Neutrokine-alpha conjugate or a Neutrokine-alpha complex of the invention may be associated with heterologous polypeptides, heterologous nucleic acids, toxins, or prodrugs via hydrophobic, hydrophilic, ionic and/or covalent interactions.

In one embodiment, the invention provides a method for the specific delivery of compositions of the invention to cells by administering a Neutrokine-alpha conjugate or a Neutrokine-alpha complex that is associated with heterologous polypeptides or nucleic acids. In one example, the invention provides a method for delivering a therapeutic protein or cytotoxin to or into the targeted cell.

In another embodiment, the invention provides for a method of killing lymphocytes, comprising, or alternatively consisting of, contacting a Neutrokine-alpha conjugate or a Neutrokine-alpha complex with lymphocytes. In a specific embodiment, the method of killing lymphocytes, comprises, or alternatively consists of, administering to an animal in which such killing is desired, a Neutrokine-alpha conjugate or a Neutrokine-alpha complex in an amount effective to kill lymphocytes. Lymphocytes include, but are not limited to, healthy and diseased cells as found present in an animal, preferably a mammal and most preferably a human, or as isolated from an animal, transformed cells, cell lines derived from the above listed cell types, and cell cultures derived from the above listed cell types. Lymphocytes may be found or isolated in, for example, different developmental stages or in resting, activated or anergic states. In preferred embodiments, the lymphocytes are B lineage cells.

In another embodiment, the invention provides a method for the specific destruction (i.e., killing) of cells (e.g., the destruction of cancer cells with lymphocyte phenotypes) by administering a Neutrokine-alpha conjugate or a Neutrokine-alpha complex in which such destruction of cells is desired. In one embodiment, the cells targeted for destruction express Neutrokine-alpha receptors on their surface. In another embodiment, the cells targeted for destruction are in proximity to cells that express Neutrokine-alpha receptors on their surface. In preferred embodiments the cells are B lineage tumors are B lineage cells.

In another embodiment, the invention provides a method for the specific destruction of lymphocytes (e.g., the destruction of tumor cells) by administering a Neutrokine-alpha conjugate or a Neutrokine-alpha complex in association with toxins or cytotoxic prodrugs.

In a specific embodiment, the invention provides a method for the specific destruction of cells of B cell lineage (e.g., B cell related leukemias or lymphomas) by administering a Neutrokine-alpha conjugate or a Neutrokine-alpha complex and a toxin or cytotoxic prodrug.

In one embodiment, the invention provides methods and compositions for inhibiting or reducing proliferation of lymphocytes, comprising, or alternatively consisting of, contacting an effective amount of a Neutrokine-alpha conjugate or a Neutrokine-alpha complex with lymphocytes, wherein the effective amount of a Neutrokine-alpha conjugate or a Neutrokine-alpha complex inhibits or reduces proliferation of lymphocytes. In another embodiment, the invention provides methods and compositions for inhibiting or reducing proliferation of lymphocytes comprising, or alternatively consisting of, administering to an animal in which such inhibition or reduction is desired, a Neutrokine-alpha conjugate or a Neutrokine-alpha complex in an amount effective to inhibit or reduce B cell proliferation. In preferred embodiments, the lymphocytes are B cells.

B cell proliferation is most commonly assayed in the art by measuring tritiated-thymidine incorporation. This and other assays are commonly known in the art and could be routinely adapted for the use of determining the effect of the Neutrokine-alpha conjugate or the Neutrokine-alpha complex on B cell proliferation.

In one embodiment, the invention provides methods and compositions for decreasing lifespan of lymphocytes, comprising, or alternatively consisting of, contacting an effective amount of a Neutrokine-alpha conjugate or a Neutrokine-alpha complex with lymphocytes, wherein the effective amount of the Neutrokine-alpha conjugate or the Neutrokine-alpha complex inhibits or reduces lifespan of lymphocytes. In another embodiments, the lymphocytes are B cells.

B cell life span may be measured using or routinely modify techniques known in the art. In one example, B cell lifespan is measured in vivo may be measured by 5-bromo-2′-deoxyuridine (BrdU) labeling experiments which are well known to one skilled in the art. BrdU is a thymidine analogue that gets incorporated into the DNA of dividing cells. Cells containing BrdU in their DNA can be detected using, for example fluorescently labeled anti-BrdU antibody and flow cytometry. Briefly, an animal is injected with BrdU in an amount sufficient to label developing B cells. Then, a sample of B cells is withdrawn from the animal, for example, from peripheral blood, and analyzed for the percentage of cells that contain BrdU. Such an analysis performed at several time points can be used to calculate the half life of B cells. Alternatively, B cell survival may be measured in vitro. For example B cells may be cultured under conditions where proliferation does not occur, (for example the media should contain no reagents that crosslink the immunoglobulin receptor, such as anti-IgM antibodies) for a period of time (usually 2-4 days). At the end of this time, the percent of surviving cells is determined, using for instance, the vital dye Trypan Blue, or by staining cells with propidium iodide or any other agent designed to specifically stain apoptotic cells and analyzing the percentage of cells stained using flow cytometry. One could perform this experiment under several conditions, such as B cells treated with Neutrokine-alpha conjugates or complexes and untreated B cells or B cells treated with an unlabelled form of Neutrokine-alpha protein (e.g. a Neutrokine-alpha trimer consisting of three subunits each comprsing amino acids 134-285 of SEQ ID NO:2) in order to determine the effects of Neutrokine-alpha proteins on B cell survival. These and other methods for determining B cell lifespan are commonly known in the art.

Treatment of Cancer

The present invention provides methods and compositions comprising a Neutrokine-alpha conjugate or a Neutrokine-alpha complex useful in the treatment of cancer, e.g. by destroying the cancerous cells and/or by inhibiting the growth, progression, and/or metastasis of cancer cells. In a preferred embodiment, the present invention provides methods and compositions comprising a Neutrokine-alpha conjugate or a Neutrokine-alpha complex useful in the treatment of lymphoma. Use of Neutrokine-alpha conjugates and Neutrokine-alpha complexes is superior to conventional radiotherapy because Neutrokine-alpha radiotherapy is targeted at Neutrokine-alpha receptor expressing cells rather than a non-specific population of cells such as rapidly dividing cells. Furthermore, the fact that Neutrokine-alpha receptors are not expressed on the earlier stages of lymphocyte development (pre-lymphocytes such as pre B-cells) suggests that patients treated with Neutrokine-alpha conjugates and/or Neutrokine-alpha complexes will be able to reconstitute the cells of their immune system faster than a patient that had undergone conventional radiotherapy.

In a preferred embodiment, the present invention provides methods and compositions comprising a Neutrokine-alpha conjugate or a Neutrokine-alpha complex useful in the treatment of non-Hodgkin's lymphoma. In particular embodiments, the non-Hodgkin's lymphoma may be a diffuse large cell, mantle cell, marginal zone, or follicular lymphoma. In preferred embodiments, Neutrokine alpha complexes of the invention comprising radiometal ions that emit beta-particles, e.g., ⁹⁰Y, are used to treat solid tumors such as lymphoma.

In a preferred embodiment, the present invention provides methods and compositions comprising a Neutrokine-alpha conjugate or a Neutrokine-alpha complex useful in the treatment of chronic lymphocytic leukemia.

In a preferred embodiment, the present invention provides methods and compositions comprising a Neutrokine-alpha conjugate or a Neutrokine-alpha complex useful in the treatment of multiple myeloma.

In other preferred embodiments, Neutrokine alpha complexes of the invention comprising radiometal ions that emit alpha-particles or auger electrons, e.g., ¹¹¹In (auger electron emitter), are used to treat diffuse cancers such as chronic lymphocytic leukemia and multiple myeloma.

A non-limiting example of a Neutrokine-alpha conjugate or a Neutrokine-alpha complex of the invention that can be administered to a cancer patient is a Neutrokine-alpha conjugate or complex of Formula IV which binds to a Neutrokine-alpha receptor.

Additional cancers that may be treated with methods and compositions comprising a Neutrokine-alpha conjugate or a Neutrokine-alpha complex include B cell malignancies such as acute lymphocytic leukemia (ALL), plasmacytomas, Burkitt's lymphoma, and EBV-transformed diseases.

Neutrokine-alpha conjugates and/or a Neutrokine-alpha complexes may be useful in the treatment of Chronic Myelogenous Leukemia by decreasing the involvement of B cells and Ig associated with this disease.

Because Neutrokine-alpha protein, e.g., amino acids 134-285 of SEQ ID NO:2, is able to stimulate B cell proliferation, activation and survival, administration of Neutrokine-alpha conjugates may induce proliferation of normal and cancerous B cells, thus making it more susceptible to Neutrokine-alpha complexes and/or other anti-neoplastic agents (e.g., chemotherapy and radiation therapy). For example, multiple myeloma is a slowly dividing disease and is thus refractory to virtually all anti-neoplastic regimens. If these cells were induced to proliferate more rapidly their susceptibility profile would likely change.

The possibility that receptors for proteins comprising Neutrokine-alpha or fragments or variants thereof (e.g., Neutrokine-alpha heterotimers (described below) comprising one or two APRIL monomers) are expressed on cells or cell lines of fibroblastic or epithelial lineage or having fibroblastic or epithelial morphology, indicates that Neutrokine-alpha conjugates and/or Neutrokine-alpha complexes of the invention may be used to diagnose or treat cancers of cells of fibroblastic or epithelial lineage or having fibroblastic or epithelial morphology (e.g., skin cancer, lung cancer, or colorectal cancer).

Additionally, because the energy from certain radioactive decay events can span more than a single cell diameter, Neutrokine-alpha conjugates and/or Neutrokine-alpha complexes of the invention may be used to kill cells, e.g., cancerous cells, in close proximity to cells that express Neutrokine alpha receptors. In specific embodiments, Neutrokine-alpha conjugates and/or Neutrokine-alpha complexes of the invention may be used to prevent the metastasis of cancers through the lymphatic system. Cancerous cells that spread though the lymphatic system are likely to be in close proximity to cells that express receptors for Neutrokine-alpha (e.g., B cells that are numerous in the lymph, lymph nodes and spleen). Thus, decay events from a Neutrokine-alpha complex of the invention associated with a B cell may be able to kill the cell expressing Neutrokine-alpha receptors, but also the cancer cell in close proximity to said cell expressing Neutrokine-alpha receptors, thereby preventing or inhibiting metastasis of said cancer through the lymphatic system.

The present invention further encompasses methods and compositions for killing cells bearing Neutrokine-alpha receptors and/or cells in close proximity to cells bearing Neutrokine-alpha receptors, comprising, or alternatively consisting of, contacting a Neutrokine-alpha conjugate and/or a Neutrokine-alpha complex of the invention with cells bearing Neutrokine-alpha receptors. In preferred embodiments, the cells bearing Neutrokine-alpha receptors are B cells.

The present invention further encompasses methods and compositions for killing cells bearing Neutrokine-alpha receptors or cells in close proximity to cells bearing a Neutrokine-alpha receptor, comprising, or alternatively consisting of, administering to an animal in which such killing is desired, a Neutrokine-alpha conjugate and/or a Neutrokine-alpha complex in an amount effective to kill cells bearing Neutrokine-alpha receptors and/or cells in close proximity to cells bearing a Neutrokine-alpha receptor. In preferred embodiments, the cells bearing Neutrokine-alpha receptors are B cells.

Treatment of Autoimmune Disease

Elevated levels of Neutrokine-alpha protein correlate with autoimmune disease (Zhang et al., The Journal of Immunology 166:6-10 (2001); Cheema et al., Arthritis and Rheumatism 44:1313-1319 (2001), Groom et al., Journal of Clinical Investigation 109:59-68 (2002); and Vaux, The Journal of Clinical Investigation 109:17-18 (2002)). Additionally, it has been demonstrated that that elevated levels of Neutrokine-alpha are relevant to the pathology of autoimmune disease because a Neutrokine-alpha antagonist (TACI-Fc) is able to ameliorate the symptoms of autoimmunity in mouse models of autoimmune disease (Gross et al., Nature, 404:995-999 (2000)).

Elevated levels of soluble Neutrokine-alpha have been observed in the serum of patients with Systemic Lupus Erythematosus (SLE). In comparing the sera of 150 SLE patients with that of 38 control individuals, it was found that most of the SLE patients had more than 5 ng/mL of serum Neutrokine-alpha, more than 30% of SLE patients had levels greater than 10 ng/mL, and approximately 10% of SLE patients had serum Neutrokine-alpha levels greater than 20 ng/mL. In contrast, the majority of normal controls had Neutrokine-alpha levels less than 5 ng/mL, and less than 10% had levels higher than 10 ng/mL. The elevated levels of Neutrokine-alpha protein in sera is present in the soluble form and has biologic activity as assayed by the ability to stimulate anti-IgM treated B cells in vitro. SLE patients with more than 15 ng/mL serum Neutrokine-alpha were also found to have elevated levels of anti-dsDNA antibodies compared to both normal controls and SLE patients with less than 5 ng/mL of serum Neutrokine-alpha (Zhang et al., The Journal of Immunology 166:6-10 (2001)).

In addition, the serum of two subgroups of patients which were positive for anti-nuclear antibodies (ANA+) but did not meet the formal requirements of the American College of Rheumatology (ACR) for classification of SLE were analyzed for Neutrokine-alpha levels. The first subgroup of sera was ANA+ sera that came from patients who did not present with the clinical impression of SLE. This group had only slightly elevated levels of Neutrokine-alpha (˜9 ng/mL Neutrokine-alpha). The second subgroup however, which was ANA+ sera from patients who presented with the clinical impression of SLE, had significantly increased Neutrokine-alpha levels (˜15 ng/mL) (See, Zhang et al., The Journal of Immunology, 166:6-10) (2001). These results suggest that an elevated level of Neutrokine-alpha precedes the formal fulfillment of the ACR criteria. The ACR criteria are described in Tan, E. M., et al., Arthritis and Rheumatism 25:1271-1277 (1982).

Therefore, in one embodiment of the present invention, a Neutrokine-alpha conjugate or a Neutrokine-alpha complex may be used to treat or ameliorate autoimmune disease. In a specific embodiment a Neutrokine-alpha conjugate or a Neutrokine-alpha complex may be used to treat or ameliorate autoimmune diseases associated with autoantibody production.

The present invention further encompasses methods and compositions for treating or ameliorating an autoimmune disease, comprising, or alternatively consisting of, contacting a Neutrokine-alpha conjugate and/or a Neutrokine-alpha complex of the invention with cells bearing Neutrokine-alpha receptors. In preferred embodiments, the cells bearing Neutrokine-alpha receptors are B cells.

The present invention further encompasses methods and compositions for treating or ameliorating an autoimmune disease, comprising, or alternatively consisting of, administering to an animal in which such treatment is desired, a Neutrokine-alpha conjugate and/or a Neutrokine-alpha complex in an amount effective to treat or ameliorate an autoimmune disease.

Autoantibody production is common to several autoimmune diseases and contributes to tissue destruction and exacerbation of disease. Autoantibodies can also lead to the occurrence of immune complex deposition complications and lead to many symptoms of systemic lupus erythematosus, including kidney failure, neuralgic symptoms and death. Decreasing or preventing antibody production would be beneficial in the treatment of autoimmune diseases such as myasthenia gravis and rheumatoid arthritis. For example, B cells have been shown to play a role in the secretion of arthritogenic immunoglobulins in rheumatoid arthritis, (Korganow et al., Immunity 10:451-61, 1999). One way to achieve the inhibition or abolition of autoantibody production, as well as of antibody production in general, is via the destruction or ablation of antibody secreting cells using a Neutrokine-alpha conjugate or a Neutrokine-alpha complex of the present invention.

In another preferred embodiment, a Neutrokine-alpha conjugate or a Neutrokine-alpha complex may be used to treat or ameliorate systemic lupus erythematosus.

In a preferred embodiment, a Neutrokine-alpha conjugate or a Neutrokine-alpha complex may be used to treat or ameliorate rheumatoid arthritis.

In another preferred embodiment, a Neutrokine-alpha conjugate or a Neutrokine-alpha complex may be used to treat or ameliorate Sjögren's syndrome.

In another preferred embodiment, a Neutrokine-alpha conjugate or a Neutrokine-alpha complex may be used to treat or ameliorate idiopathic thrombocytopenic purpura (ITP).

In another preferred embodiment, a Neutrokine-alpha conjugate or a Neutrokine-alpha complex may be used to treat or ameliorate IgA nephropathy.

In another preferred embodiment, a Neutrokine-alpha conjugate or a Neutrokine-alpha complex may be used to treat or ameliorate Myasthenia gravis.

In another preferred embodiment, a Neutrokine-alpha conjugate or a Neutrokine-alpha complex may be used to treat or ameliorate vasculitis.

A non-limiting example of a Neutrokine-alpha conjugate or a Neutrokine-alpha complex of the invention that can be administered to an individual with an autoimmune disease is a Neutrokine-alpha conjugate or complex of Formula IV which binds to a Neutrokine-alpha receptor.

Neutrokine-alpha conjugates and/or a Neutrokine-alpha complexes may be useful in the treatment of chronic hypergammaglobulinemia evident in such diseases as monoclonalgammopathy of undetermined significance (MGUS), Waldenstrom's disease, related idiopathic monoclonalgammopathies, and plasmacytomas.

More generally, the invention provides methods and compositions for inhibiting or reducing immunoglobulin production (e.g., IgM, IgG, and/or IgA production), comprising, or alternatively consisting of, contacting an effective amount of Neutrokine-alpha complex or Neutrokine-alpha conjugate with lymphocytes, wherein the effective amount of Neutrokine-alpha complex or Neutrokine-alpha conjugate inhibits or reduces immunoglobulin production.

In specific embodiments, the invention provides methods and compositions for inhibiting or reducing immunoglobulin production (e.g., IgM, IgG, and/or IgA production) in response to T cell dependent antigens, comprising, or alternatively consisting of, contacting an effective amount of Neutrokine-alpha complex or Neutrokine-alpha conjugate with lymphocytes, wherein the effective amount of Neutrokine-alpha complex or Neutrokine-alpha conjugate inhibits or reduces immunoglobulin production in response to T cell dependent antigens.

In specific embodiments, the invention provides methods and compositions for inhibiting or reducing immunoglobulin production (e.g. IgM, IgG, and/or IgA production) in response to T cell independent antigens, comprising, or alternatively consisting of, contacting an effective amount of Neutrokine-alpha complex or Neutrokine-alpha conjugate with lymphocytes, wherein the effective amount of Neutrokine-alpha complex or Neutrokine-alpha conjugate inhibits or reduces immunoglobulin production in response to T cell independent antigens.

In another embodiment, the invention provides methods and compositions for inhibiting or reducing immunoglobulin production (e.g., IgM, IgG, and/or IgA production), comprising, or alternatively consisting of, administering to an animal in which such inhibition or reduction is desired, a Neutrokine-alpha conjugate or a Neutrokine-alpha complex in an amount effective to inhibit or reduce immunoglobulin production.

In another embodiment, the invention provides methods and compositions for inhibiting or reducing immunoglobulin production (e.g. IgM, IgG, and/or IgA production) in response to T cell dependent antigens, comprising, or alternatively consisting of, administering to an animal in which such inhibition or reduction is desired, a Neutrokine-alpha conjugate or a Neutrokine-alpha complex in an amount effective to inhibit or reduce immunoglobulin production in response to T cell dependent antigens.

In another embodiment, the invention provides methods and compositions for inhibiting or reducing immunoglobulin production (e.g. IgM, IgG, and/or IgA production) in response to T cell independent antigens, comprising, or alternatively consisting of, administering to an animal in which such inhibition or reduction is desired, a Neutrokine-alpha conjugate or a Neutrokine-alpha complex in an amount effective to inhibit or reduce immunoglobulin production in response to T cell independent antigens.

Determinations of immunoglobulin levels are most often performed by comparing the level of immunoglobulin in a sample to a standard containing a known amount of immunoglobulin using ELISA assays. Determination of immunoglobulin levels in a given sample, can readily be determined using ELISA or other methods known in the art.

Additional autoimmune disorders and conditions associated with these disorders that may be treated, prevented, and/or diagnosed with the a Neutrokine-alpha conjugate or a Neutrokine-alpha complex include, but are not limited to, autoimmune hemolytic anemia, autoimmune neutropenia, autoimmune neonatal thrombocytopenia, idiopathic thrombocytopenia purpura, autoimmunocytopenia, hemolytic anemia, antiphospholipid syndrome, dermatitis, allergic encephalomyelitis, myocarditis, relapsing polychondritis, rheumatic heart disease, glomerulonephritis (e.g., IgA nephropathy), dense deposit disease, Multiple Sclerosis, Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, Guillain-Barre Syndrome, gluten sensitive enetropathy, insulin dependent diabetes mellitus, discoid lupus, and autoimmune inflammatory eye disease.

Additional autoimmune disorders that may be treated, prevented, and/or diagnosed with the compositions of the invention include, but are not limited to, autoimmune thyroiditis, hypothyroidism (i.e., Hashimoto's thyroiditis) (often characterized, e.g., by cell-mediated and humoral thyroid cytotoxicity), systemic lupus erhythematosus (often characterized, e.g., by circulating and locally generated immune complexes), Goodpasture's syndrome (often characterized, e.g., by anti-basement membrane antibodies), Pemphigus (often characterized, e.g., by epidermal acantholytic antibodies), Receptor autoimmunities such as, for example, (a) Graves' Disease (often characterized, e.g., by TSH receptor antibodies), (b) Myasthenia Gravis (often characterized, e.g., by acetylcholine receptor antibodies), and (c) insulin resistance (often characterized, e.g., by insulin receptor antibodies), autoimmune hemolytic anemia (often characterized, e.g., by phagocytosis of antibody-sensitized RBCs), autoimmune thrombocytopenic purpura (often characterized, e.g., by phagocytosis of antibody-sensitized platelets.

Additional autoimmune disorders that may be treated, prevented, and/or diagnosed with the compositions of the invention include, but are not limited to, rheumatoid arthritis (often characterized, e.g., by immune complexes in joints), schleroderma with anti-collagen antibodies (often characterized, e.g., by nucleolar and other nuclear antibodies), mixed connective tissue disease (often characterized, e.g., by antibodies to extractable nuclear antigens (e.g., ribonucleoprotein)), polymyositis/dermatomyositis (often characterized, e.g., by nonhistone ANA), pernicious anemia (often characterized, e.g., by antiparietal cell, microsomes, and intrinsic factor antibodies), idiopathic Addison's disease (often characterized, e.g., by humoral and cell-mediated adrenal cytotoxicity, infertility (often characterized, e.g., by antispermatozoal antibodies), glomerulonephritis (often characterized, e.g., by glomerular basement membrane antibodies or immune complexes) such as primary glomerulonephritis and IgA nephropathy, bullous pemphigoid (often characterized, e.g., by IgG and complement in basement membrane), Sjogren's syndrome (often characterized, e.g., by multiple tissue antibodies, and/or a specific nonhistone ANA (SS-B)), diabetes mellitus (often characterized, e.g., by cell-mediated and humoral islet cell antibodies), and adrenergic drug resistance (including adrenergic drug resistance with asthma or cystic fibrosis) (often characterized, e.g., by beta-adrenergic receptor antibodies).

Additional autoimmune disorders that may be treated, prevented, and/or diagnosed with the compositions of the invention include, but are not limited to, chronic active hepatitis (often characterized, e.g., by smooth muscle antibodies), primary biliary cirrhosis (often characterized, e.g., by mitchondrial antibodies), other endocrine gland failure (often characterized, e.g., by specific tissue antibodies in some cases), vitiligo (often characterized, e.g., by melanocyte antibodies), vasculitis (often characterized, e.g., by Ig and complement in vessel walls and/or low serum complement), post-MI (often characterized, e.g., by myocardial antibodies), cardiotomy syndrome (often characterized, e.g., by myocardial antibodies), urticaria (often characterized, e.g., by IgG and IgM antibodies to IgE), atopic dermatitis (often characterized, e.g., by IgG and IgM antibodies to IgE), asthma (often characterized, e.g., by IgG and IgM antibodies to IgE), inflammatory myopathies, and many other inflammatory, granulomatous, degenerative, and atrophic disorders.

In another embodiment, the autoimmune diseases and disorders and/or conditions associated with the diseases and disorders recited above are treated, prevented, and/or diagnosed using a Neutrokine-alpha conjugate or a Neutrokine-alpha complex of the invention.

Additional uses

The present invention further encompasses methods and compositions for treating or diseases and disorders, particularly those described herein, comprising, or alternatively consisting of, contacting a Neutrokine-alpha conjugate and/or a Neutrokine-alpha complex of the invention with cells bearing Neutrokine-alpha receptors. In preferred embodiments, the cells bearing Neutrokine-alpha receptors are B cells.

The present invention further encompasses methods and compositions for treating or ameliorating diseases and disorders, particularly those described herein, comprising, or alternatively consisting of, administering to an animal in which such treatment is desired, a Neutrokine-alpha conjugate and/or a Neutrokine-alpha complex in an amount effective to treat or ameliorate an autoimmune disease.

Neutrokine-alpha conjugates and/or Neutrokine-alpha complexes may also be useful for the treatment of athersclerosis.

Neutrokine-alpha conjugates and/or Neutrokine-alpha complexes may also be useful for the treatment of asthma and other chronic airway diseases such as bronchitis and emphysema.

A Neutrokine-alpha conjugate or a Neutrokine-alpha complex may be used to modulate IgE concentrations in vitro or in vivo.

Additionally, a Neutrokine-alpha conjugate or a Neutrokine-alpha complex may be used to treat, prevent, and/or diagnose IgE-mediated allergic reactions. Such allergic reactions include, but are not limited to, asthma, rhinitis, and eczema.

Neutrokine-alpha conjugates and/or a Neutrokine-alpha complexes may be useful as an immunosuppressive agent.

A Neutrokine-alpha conjugate or a Neutrokine-alpha complex may be used to treat, prevent, and/or diagnose various immune system-related disorders and/or conditions associated with these disorders, in mammals, preferably humans. Many autoimmune disorders result from inappropriate recognition of self as foreign material by immune cells. This inappropriate recognition results in an immune response leading to the destruction of the host tissue. Therefore, the administration of a Neutrokine-alpha conjugate or a Neutrokine-alpha complex that can inhibit an immune response, particularly the proliferation of B cells and/or the production of immunoglobulins, may be an effective therapy in treating and/or preventing autoimmune disorders. Thus, in preferred embodiments, a Neutrokine-alpha conjugate or a Neutrokine-alpha complex of the invention is used to treat, prevent, and/or diagnose an autoimmune disorder.

In another embodiment, AIDS is treated, prevented, and/or diagnosed using a Neutrokine-alpha conjugate or a Neutrokine-alpha complex.

In another embodiment, HIV infection is treated, prevented, and/or diagnosed using a Neutrokine-alpha conjugate or a Neutrokine-alpha complex.

A Neutrokine-alpha conjugate or a Neutrokine-alpha complex may also be employed to inhibit T-cell proliferation by the inhibition of IL-2 biosynthesis for the treatment of T-cell mediated autoimmune diseases.

All of the above described applications also apply to veterinary medicine.

The above-recited applications have uses in a wide variety of hosts. Such hosts include, but are not limited to, human, murine, rabbit, goat, guinea pig, camel, horse, mouse, rat, hamster, pig, micro-pig, chicken, goat, cow, sheep, dog, cat, non-human primate, and human. In specific embodiments, the host is a mouse, rabbit, goat, guinea pig, chicken, rat, hamster, pig, sheep, dog or cat. In preferred embodiments, the host is a mammal. In most preferred embodiments, the host is a human.

Formulation and Administration

The timing of the administration can vary substantially. In one embodiment, the entire dose is provided in a single bolus. Alternatively, the dose can be provided by multiple administrations, such as an extended infusion method or by repeated injections administered over a span of weeks, for example six to twelve weeks between radioimmunotherapeutic doses. In one embodiment, the Neutrokine-alpha complex is administered at two week intervals. In another embodiment, the Neutrokine-alpha complex is administered over a span of about 2 to about 4 days.

Alternatively, a slow intravenous infusion of the complex may be administered, having a period for the infusion of about one to about 24 hours.

In one embodiment of the treatment of B-cell lymphoma, intravenous administration is utilized to deliver the Neutrokine-alpha complex to the site of the tumor. In another embodiment, intralymphatic routes of administration, such as subcutaneous or intramuscular injection or by catheterization of lymphatic vessels, are utilized.

In one embodiment, a treatment regimen comprises two dosages of a Neutrokine-alpha conjugate or Neutrokine-alpha complex. The first dose is an imaging dose. The second dose is a therapeutic dose. The imaging dose can be used to confirm that the Neutrokine-alpha conjugate or Neutrokine-alpha complex localizes to specific organs, tissues, and/or cells. Upon confirmation, or sometime thereafter, that the Neutrokine-alpha conjugate or Neutrokine-alpha complex localizes to certain organs, tissues, and/or cells, the second dose comprising a therapeutically effective amount of a Neutrokine-alpha conjugate or Neutrokine-alpha complex is administered to the subject.

In one embodiment, an imaging dose of a Neutrokine-alpha complex comprising a ion suitable for imaging, e.g., ¹¹¹In, is administered to a subject. The distribution of the Neutrokine-alpha complex is then assessed. In one embodiment, a first image of the of the subject is obtained from about 0.5 to about 24 hours after administration of the Neutrokine-complex. In another embodiment, a second image is obtained of the subject from about 24 hours to about 72 hours after administration of the Neutrokine-alpha complex. In a further embodiment, a third image is obtained of the subject from about 72 hours to about 120 hours after administration of the Neutrokine-alpha complex. After the first, second, and/or third images are obtained, the biodistribution is determined and analyzed, and, if the biodistribution is deemed acceptable, a therapeutic dose of a Neutrokine-alpha complex comprising a ion suitable for therapy, e.g., ⁹⁰Y, is administered to said subject.

Various delivery systems are known and can be used to administer a Neutrokine-alpha conjugate or a Neutrokine-alpha complex of the invention, e.g., encapsulation in liposomes, microparticles, microcapsules, receptor-mediated endocytosis (see, e.g., Wu and Wu, J. Biol. Chem. 262:4429-4432 (1987)), etc. Methods of introduction include but are not limited to intradermal, intramuscular, intraperitoneal, intravenous, subcutaneous, intranasal, epidural, and oral routes. The Neutrokine-alpha conjugate, Neutrokine-alpha complex, or a composition thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the Neutrokine-alpha conjugate or Neutrokine-alpha complex or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the Neutrokine-alpha conjugate or the Neutrokine-alpha complex or composition can be delivered in a vesicle, in particular a liposome (see Langer, Science 249:1527-1533 (1990); Treat et al., in Liposomes in therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the Neutrokine-alpha conjugate or the Neutrokine-alpha complex or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989);

Howard et al., J. Neurosurg. 71:105 (1989)). In yet another embodiment, a controlled release system can be placed in proximity of therapeutic target, e.g., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

When treating a subject with cancer, the Neutrokine-alpha conjugate or the Neutrokine-alpha complex may be used in conjunction with additional chemotherapeutic agents that are useful for the treatment of cancer. In particular, in one embodiment of the present invention, the Neutrokine-alpha complex or Neutrokine-alpha conjugate is administered, to patient in need of such treatment, along with one or more chemotherapeutic agents. In a further embodiment, one or more chemotherapeutic agents are selected from the group consisting of chemotherapeutic agents used to treat or prevent one or more conditions selected from the group consisting of non-Hodgkin's lymphoma, chronic lymphocytic leukemia, multiple myeloma. Such agents are well-known in the art.

When treating a subject with autoimmune disease, the Neutrokine-alpha conjugate or the Neutrokine-alpha complex may be used in conjunction with additional agents that are useful for the treatment of autoimmune diseases. In particular, in one embodiment of the present invention, the Neutrokine-alpha complex or Neutrokine-alpha conjugate is administered, to patient in need of such treatment, along with one or more immunosuppressants. In a further embodiment, one or more chemotherapeutic agents are selected from the group consisting of chemotherapeutic agents used to treat or prevent one or more conditions selected from the group consisting of systemic lupus erythrematosus, rheumatoid arthritis, multiple sclerosis, Crohn's disease, diabetes, Wegener's granulomatous, myasthenia gravis, and asthma. Such agents are well-known in the art.

The radiometric dosage to be applied can vary substantially. The Neutrokine-alpha complex or a composition comprising a Neutrokine-alpha conjugate and a radionuclide can be administered at a dose of about 0.1 to about 100 mCi per 70 kg body weight. In another embodiment, the Neutrokine-alpha complex or a composition comprising a Neutrokine-alpha conjugate and a radionuclide can be administered at a dose of about 0.1 to about 50 mCi per 70 kg body weight. In another embodiment, the Neutrokine-alpha complex or a composition comprising a Neutrokine-alpha conjugate and a radionuclide can be administered at a dose of about 0.1, 0.5, 1, 5, 10, 15, 20, 25, 30, 35, 40, 50, 60, 70, 80, 90 or 100 mCi per 70 kg body weight.

For example, lymphomas are known to be radiosensitive tumors. For immunodiagnostic imaging, trace-labeling by the complex may be used, typically 1-20 mg of Neutrokine-alpha protein is labeled with about 1 to 60 mCi of radioisotope. The dose may be somewhat dependent upon the isotope used for imaging; amounts in the higher end of the range, preferably 40 to 60 mCi, may be used with ^{99m}Tc; amounts in the lower end of the range, preferably 1-20 mCi, may be used with ¹¹¹In. For imaging purposes, about 1 to about 30 mg of Neutrokine-alpha complex can be given to the subject. For radioimmunotherapeutic purposes, the Neutrokine-alpha complex is administered to a subject in sufficient amount so that the whole body dose received is up to about 1100 cGy, but preferably less than or equal to 500 cGy.

The total amount of Neutrokine-alpha protein administered to a subject, including Neutrokine-alpha protein, Neutrokine-alpha conjugate and Neutrokine-alpha complex, can range from 1.0 μg/kg to 1.0 mg/kg of patient body weight. In another embodiment, total amount of Neutrokine-alpha protein administered to a subject, can range from 20 μg/kg to 100 μg/kg of patient body weight.

An amount of radioactivity which would provide approximately 500 cGy to the whole body of a human is estimated to be about 825 mCi of ¹³¹I. The amounts of radioactivity to be administered depend, in part, upon the isotope chosen. For ⁹⁰Y therapy, from about 1 to about 200 mCi amounts of radioactivity are considered appropriate, with preferable amounts being 1 to 150 mCi, and 1 to 100 mCi (e.g., 60 mCi) being most preferred. The preferred means of estimating tissue doses from the amount of administered radioactivity is to perform an imaging or other pharmacokinetic regimen with a tracer dose, so as to obtain estimates of predicted dosimetry. In determining the appropriate dosage of radiopharmaceutical to administer to an individual, it is necessary to consider the amount of radiation that individual organs will receive compared to the maximum tolerance for such organs. Such information is known to those skilled in the art, for example, see Emami et al., International Journal of Radiation Oncology, Biology, Physics 21:109-22 (1991); and Meredith, Cancer Biotherapy & Radiopharmaceuticals 17:83-99 (2002), both of which are hereby incorporated by reference in their entireties.

A “high-dose” protocol, for example in the range of 200 to 600 cGy (or higher) to the whole body, may require the support of a bone-marrow replacement protocol, as the bone-marrow is the tissue which limits the radiation dosage due to toxicity.

The composition to be administered may be given in a single treatment or fractionated into several portions and administered at different times. Administering the composition in fractionated doses may make it possible to minimize certain damage to non-target tissue. Such multiple dose administration may be more effective.

The present method may further comprise administering a second composition comprising one or more basic amino acids, wherein said second composition is administered prior to the Neutrokine-alpha complex, Neutrokine-alpha conjugate, or Neutrokine-alpha composition. Said second composition reduces renal accumulation of radioactivity.

The Neutrokine-alpha complex according to the present invention may be used as an imaging agent. Imaging agents are useful in a number of applications, include planar imaging, magnetic resonance imaging (MRI) applications, ultrasound imaging applications, and X-ray applications. Other applications in which imaging agents are useful include scintigraphic, positron emission tomography (PET), single photon emission computed tomography (SPECT), gamma scintigraphy, electrical impedance, light, or magnetometric imaging applications.

When used as an imaging agent, the Neutrokine-alpha complex may contain any suitable metal ion in accordance with the invention. In one embodiment, the imaging agents of the invention contain radionuclides suitable for use in PET or SPECT imaging. In another embodiment, the radionuclide used in the imaging agent is a radionuclide selected from the group consisting of ^{99m}Tc, ⁶⁸Ga, ⁶²Cu, and ¹¹¹In. In another embodiment, the radionuclide is ¹¹¹In.

The Neutrokine-alpha complex of the present invention may be administered to patients for imaging in amounts sufficient to yield the desired contrast with the particular imaging technique. Generally, dosages of from about 0.001 to about 5.0 mmoles of chelated imaging metal ion per kilogram of patient bodyweight are effective to achieve adequate contrast enhancements. For most MRI applications, preferred dosages of imaging metal ion will be in the range of from about 0.02 to about 1.2 mmoles/kg bodyweight while, for X-ray applications, dosages of from about 0.05 to about 2.0 mmoles/kg are generally effective to achieve X-ray attenuation. Preferred dosages for most X-ray applications are from about 0.1 to about 1.2 mmoles of the lanthanide or heavy metal compound/kg bodyweight. Where the chelated species is a radionuclide, dosages of about 0.01 to about 100 mCi, preferably 0.1 to 50 mCi, will normally be sufficient per 70 kg bodyweight.

Another embodiment of the present invention is a method of imaging the site of infection or inflammation in a patient comprising administering a Neutrokine-alpha-complex or a composition comprising a Neutrokine-alpha conjugate and a metal ion to a patient, preferably by injection or infusion; and imaging the patient, preferably by using either planar or SPECT gamma scintigraphy.

Another embodiment of the present invention is a method of imaging the site of cancer in a patient comprising administering a Neutrokine-alpha conjugate or Neutrokine-alpha complex to a patient by injection or infusion; and imaging the patient using either planar or SPECT gamma scintigraphy.

The Neutrokine-alpha complex of the present invention may be used as a contrast agent.

Also in accordance with the present invention, a method for diagnostic examination or therapeutic treatment of a mammal is provided. This method is based on the mechanism of receptor-mediated endocytosis activity and involves i) the movement of a Neutrokine-alpha complex that can be detected by external imaging techniques into the interior of a cell through invagination of the cell membrane. The Neutrokine-alpha protein serves to deliver the chelated metal or a chemotherapy agent into a cell that expresses Neutrokine-alpha binding protein or a Neutrokine alpha receptor, thereby enabling diagnostic examination, radiotherapy or chemotherapeutic treatment of an organ or tissue comprising the cell.

In one embodiment, the method of the present invention comprises the steps of (a) administering to a subject, preferably a mammal, a composition comprising a Neutrokine-alpha conjugate or a Neutrokine-alpha complex and a pharmaceutically acceptable carrier and (b) monitoring the biodistribution of a metal ion. Said metal ion can be administered as part of the complex or separately as a solution.

Paramagnetic metals are used in affecting the relaxation times of nuclei in mammalian tissue. This is useful for magnetic resonance imaging processes. On the basis of differences in proton density and relaxation times, images of biological tissues can be obtained which may be used for diagnostic purposes. The greater the differences in the relaxation times of the nuclei which are present in the tissues being examined, the greater will be the contrast in the image that is obtained.

It is known that the relaxation times of neighboring nuclei can be affected by the use of paramagnetic salts. In solution, the paramagnetic salts are toxic in mammals. Hence, to reduce the toxic effect of paramagnetic metal ions administered for diagnostic purposes, they are combined with complex compounds, i.e., chelating agents. The Neutrokine-alpha complex increases the concentration of the metal at locations containing a site with affinity for Neutrokine-alpha, for example, a B-cell containing a Neutrokine-alpha receptor (e.g., BAFF receptor, TACI receptor, or BCMA receptor), thus providing increased contrast of the tissue comprising said site.

Doses for administration of paramagnetic metals in the complex of the present invention can be from about 0.05 to about 0.3 mmol/kg of body weight.

The metal complexes of the present invention find utility as diagnostic and/or therapeutic agents. Thus, the present invention provides methods for the diagnosis of the presence and/or status of a disease state, or for the treatment of a disease state, comprising the step of administering a metal complex of the present invention to a subject in need thereof. The metal complexes of the present invention may be administered by an appropriate route such as orally, parentally (for example, intravenously), intramuscularly or intraperitoneally or by any other suitable method. For example, the complexes of this invention may be administered to a subject by bolus or slow infusion intravenous injection.

In accordance with the present invention, the Neutrokine-alpha conjugate, Neutrokine-alpha complex, and pharmaceutical compositions of the present invention can be administered by any means that achieve their intended purpose. For example, administration can be by subcutaneous, intravenous, intramuscular, intraperitoneal, buccal, or ocular routes, rectally, parenterally, intrasystemically, intravaginally, topically (as by powders, ointments, drops or transdermal patch), or as an oral or nasal spray. The dosage administered will be dependent upon the age, health, and weight of the recipient, kind of concurrent treatment, if any, frequency of treatment, and the nature of the effect desired.

The effective dose of radiation or metal content to be utilized for any application will also depend upon the particulars of that application. In treating tumors, for example, the dose will depend, inter alia, upon tumor burden, accessibility and the like. Somewhat similarly, the use of metal chelate conjugated antibodies for diagnostic purposes will depend, inter alia, upon the sensing apparatus employed, the location of the site to be examined, and other similar factors.

Certain embodiments of the invention can be practiced either with scintigraphic or magnetic resonance imaging agents. A combination of these imaging agents can also be used, although this requires more complex instrumentation and data processing. Scintigraphic imaging according to the method of the invention is effected by obtaining a scintigram of the tissue or organ of interest, using as an imaging agent a Neutrokine-alpha conjugate or Neutrokine-alpha complex. The scintigram is normally taken by a gamma imaging camera having one or more windows for detection of energies in the50-500 keV range. Use of radioisotopes with higher energy, beta, or positron emissions would entail use of imaging cameras with the appropriate detectors, all of which are conventional in the art. The scintigraphic data can be stored in a computer for later processing.

Neutrokine-alpha conjugates and Neutrokine-alpha complexes of the invention may be administered alone or in combination with each other or other agents. In preferred embodiments, Neutrokine-alpha conjugates and Neutrokine-alpha complexes of the invention may be administered alone or in combination with Neutrokine-alpha protein.

In other embodiments, Neutrokine-alpha conjugates and Neutrokine-alpha complexes of the invention may be in combination with other chemotherapeutic agents or regimens.

In other embodiments, Neutrokine-alpha conjugates and Neutrokine-alpha complexes of the invention may be in combination with toxins or cytotoxic prodrugs. By “toxin” is meant compounds that bind and activate endogenous cytotoxic effector systems, radioisotopes, holotoxins, modified toxins, catalytic subunits of toxins, cytotoxins (cytotoxic agents), or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. “Toxin” also includes a cytostatic or cytocidal agent, a therapeutic agent or a radioactive metal ion, e.g., alpha-emitters such as, for example, ²¹³Bi, or other radioisotopes such as, for example, ¹⁰³Pd, ¹³³Xe, ¹³¹I, ⁶⁸G, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ¹¹⁷Sn, ¹⁸⁶Rhenium, ¹⁶⁶Ho, and ¹⁸⁸Rhenium; luminescent labels, such as luminol; and fluorescent labels, such as fluorescein and rhodamine, and biotin.

A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

By “cytotoxic prodrug” is meant a compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

Kits

For radiopharmaceutical or radiotherapy applications it is convenient to prepare the complexes of the present invention at, or near, the site where they are to be used. A single- or multi-vial kit comprising all or some of the components needed to prepare the complexes of this invention, other than the radionuclide ion itself, is an additional part of this invention. In one embodiment, the kit comprises all of the components for preparing a Neutrokine-alpha complex, other than the radionuclide itself.

The present invention also provides a kit comprising one or more containers, wherein each of said containers is filled with one or more of the ingredients of the pharmaceutical composition of the invention. Optionally, associated with said one or more containers is a set of written materials in the form prescribed by a governmental agency regulating the manufacture, use, or sale of pharmaceuticals or biological products, said notice reflects approval by the agency of manufacture, use or sale for human administration. The pharmaceutical kit optionally further comprises instructions to prepare said pharmaceutical compositions.

In one embodiment, a kit comprises a first container, said first container containing a Neutrokine-alpha protein, and a second container, said second container containing a chelator. Optionally, associated with said kit is a set of instructions providing the appropriate guidance on how to prepare Neutrokine-alpha conjugate using the contents of the kit.

In another embodiment, a kit comprises a first container, said first container containing a Neutrokine-alpha conjugate, and optionally associated with said kit is a set of instructions providing the appropriate guidance on how to prepare Neutrokine-alpha complex using the contents of the kit.

In another embodiment of the present invention, the kit comprises a first container, the contents of said container comprising a buffer, preferably an acetate buffer with concentration from about 10 mM to about 100 mM, preferably about 50 mM; a second container, the contents of said second container comprising a radionuclide, preferably a radionuclide selected from the group consisting of ⁹⁰Y and ¹¹¹In, wherein said radionuclide preferably is in solution, with a concentration of about 20 mCi/mL to about 200 mCi/mL, preferably about 80 mCi/mL; and a third container, the contents of said third container comprising a Neutrokine-alpha conjugate, preferably a Neutrokine-alpha conjugate which comprises a Neutrokine-alpha protein that has the sequence of SEQ ID NO:3 and a chelator that is α-(5-isothiocyanato-2-methoxyphenyl)-1,4,7,10-tetraazacyclododecane-1,4,7,10-tetraacetic acid, wherein said conjugate is preferably in solution having a concentration of about 1 mg/mL to about 10 mg/mL, preferably about 2 mg/mL. The kit further optionally comprises a fourth container, the contents of said fourth container comprising a buffer, preferably an acetate buffer having a concentration of about 1 to about 50 mM, preferably about 10 mM, having a NaCl concentration of about 1 to about 500 mM, preferably about 140 mM, having a human serum albumin (HSA) concentration of about 1% to about 20%, preferably about 7% to about 8%, more preferably about 7.5%, having a pH of about 3-8, preferably about 6, and having a MeO-DOTA-glycine concentration of about 0.01 mM to about 100 mM, preferably about 1 mM.

In another embodiment of the present invention, the kit according to the present invention optionally comprises written material describing the use of a Neutrokine-alpha complex, a Neutrokine-alpha conjugate, or a composition comprising said conjugate or complex in a radioimmunodiagnostic and/or radioimmunotherapeutic protocol. The written material can be applied directly to a container, such as by applying a label directly to a vial containing said conjugate or complex. Alternatively, said a first container holding said conjugate or complex can be placed in a second container, such as a box, and the written material, in the form of a packaging insert, can be placed in the second container together with the first container holding said conjugate or complex.

The written portion of the article of manufacture may describe indications for prescribing the conjugate or complex. Such indications could be, for example, presentation of lymphoma at any site in the body. The written material could further describe that the conjugate or complex is useful for the treatment of lymphoma or other neoplasm clonally derived from a cell of B cell lineage, indicated as set forth above. In a preferred embodiment of the invention, the written material describes a Neutrokine-alpha complex to be used in the treatment, wherein said complex comprises a DOTA chelator, a Neutrokine-alpha protein, and a radionuclide selected from the group consisting of ⁹⁰Y, ^{99m}Tc, ¹¹¹In, ⁴⁷Sc, ⁶⁷Ga, ⁵¹Cr, ^{117m}Sn, ⁶⁷Cu, ¹⁶⁷Tm, ⁹⁷Ru, ¹⁸⁸Re, ¹⁷⁷Lu, ¹⁹⁹Au, ⁴⁷Sc, ⁶⁷Ga, ⁵¹Cr, ^{177m}Sn, ⁶⁷Cu, ¹⁶⁷Tm, ⁹⁵Ru, ¹⁸⁸Re, ¹⁷⁷Lu, ¹⁹⁹Au, ²⁰³Pb, and ¹⁴¹Ce, more preferably a selected from the group consisting of ⁹⁰Y, ¹¹¹In, ¹⁷⁷Lu, ¹⁶⁶Ho, ²¹⁵Bi, and ²²⁵Ac. In a most preferred embodiment, the written material will describe that the Neutrokine-alpha conjugate or Neutrokine-alpha complex is used in the treatment of lymphoma. In other preferred embodiments, the written material describes a Neutrokine-alpha complex to be used in the treatment, wherein said complex comprises a MeO-DOTA-NCS chelator, a Neutrokine-alpha protein, and a radionuclide selected from the group consisting of a ⁹⁰Y and ¹¹¹In. Still further, the written material can describe that the appropriate radiometric dose to be administered for an immunodiagnostic scanning is provided by 1 to 35 mCi of radioisotope, while the appropriate dose for therapeutic administration should be below 150 cGy to the whole body if bone marrow replacement support cannot be provided, but can be as high as 600 cGy to the whole body if bone marrow replacement support is provided. The doses for particular isotopes, especially as set forth herein below, might also be described.

The written material is preferably provided in the form required by the Food and Drug Administration for a package insert for a prescription drug. The written material may indicate that the antibody would be prescribed for use in patients having a diagnosis of B cell lymphoma and can be administered to patients presenting lymphoma in any site in the body. The written material may indicate that the conjugate or complex as described herein is useful as an initial or secondary treatment or in combination with other treatments.

The written material may also describe any possible side effects. The written material may also describe any possible contraindications.

The written material also may indicate that general radiologic and nuclear medicine precautions appropriate to the isotope used for labeling the antibody should be observed.

Assays

The invention also provides a method of screening a Neutrokine-alpha conjugate or Neutrokine-alpha complex to identify a complex or conjugate which binds to a Neutrokine-alpha receptor.

In the assay of the invention for a complex or conjugate that binds a Neutrokine-alpha receptor (e.g., BAFF-R (SEQ ID NO:5), TACI (SEQ ID NO:7), and BCMA (SEQ ID NO:9), a cellular compartment, such as a membrane or a preparation thereof, may be prepared from a cell that expresses a Neutrokine-alpha receptor. Exemplary cell lines that may be used for this purpose include, but are not limited to B lineage immortalized hematopoietic cell lines such as IM-9 (ATCC CCL-159); Reh (ATCC CRL-8286); ARH-77 (ATCC CRL-1621); Raji (ATCC-CCL-86); Namalwa (CRL-1432); and RPMI 8226 (ATCC CCL-155). The preparation is incubated with labeled Neutrokine-alpha (e.g., fluorescently labeled Neutrokine-alpha or radiolabeled Neutrokine-alpha) in the absence or the presence of the candidate conjugate or complex. The ability of the candidate conjugate or complex to bind the Neutrokine-alpha receptor is reflected in decreased binding of the labeled Neutrokine-alpha. Alternatively, a similar assay may be performed on live intact cells, with the final detection step comprising flow cytometry analysis.

Additionally assays which measure the direct binding of Neutrokine-alpha conjugates or complexes to one or more Neutrokine-alpha receptors. One example of such an assay is one that would utilize surface plasmon resonance technology (Biacore, Piscataway, N.J.) to analyze interactions between biomolecules. Biacore assays are well known and routine to those of skill in the art.

Definitions

The term “Neutrokine-alpha conjugate,” when used herein, means a Neutrokine-alpha protein that is covalently bonded to a chelator molecule.

The term “Neutrokine-alpha complex,” when used herein, means a Neutrokine-alpha conjugate that is associated with a radionuclide, metal ion, or other ion that is able to chelate with the chelator of said conjugate.

The term “chelator,” as used herein, refers a molecule or molecular fragment, at least part of which is able to associate with, or chelate, a metal ion. As used herein, a chelator molecule may optionally contain a linker group which connects the metal chelating portion of the chelator to the Neutrokine-alpha protein. Chelators containing such linking moieties are well known in the art. See, for example, Saji, J. “Targeted Delivery of Radiolabeled Imaging and Therapeutic Agents: Bifunctional Radiopharmaceuticals,” Crit. Rev. Therap. Drug Carrier Systems 16:209-244 (1999); Liu, S. et al., “Bifunctional Chelators for Therapeutic Lanthanide Radiopharmaceuticals,” Bioconjugate Chem. 12:7-34 (2001); Kirk-Othmer Concise Encyclopedia of Chemical Technology, pages 242-244 (John Wiley & Sons, Inc. 1985); and Van Nostrand's Scientific Encyclopedia, pages 613-615, Editor: Douglas M. Considine, P. E. (Eighth Edition, Van Nostrand Reinhold, 1995), each of which is hereby incorporated by reference in its entirety. The term “chelator moiety” or “chelating moiety” refers to the portion of the chelator molecule which forms a noncovalent interaction with a metal ion.

The term “MeO-DOTA-NCS” as used herein refers to the compound α-(5-isothiocyanato-2-methoxyphenyl)-1,4,7,10-tetraazacyclododecane-1,4,7,10-tetraacetic acid having the CAS registry number 130707-79-8.

The term “MeO-DOTA-glycine,” as used herein, refers to the compound formed by reacting MeO-DOTA-NCS with glycine.

An “effective amount” of the formulation is used for therapy. As is known by one of ordinary skill in the art, the exact amount which constitutes an effective amount may vary from one situation to another. As used herein, the term “effective amount” refers to a dosage or amount of a conjugate, complex, or composition of the present invention, said dosage or amount being sufficient to effect a sufficient or significant, intended result. For example, the amount of a Neutrokine-complex used for imaging may, and probably will, be different than the amount used to treat a B-cell mediated condition. Furthermore, the exact amount of a Neutrokine-alpha complex used to treat non-Hodgkin's lymphoma may be different than the amount used to treat multiple myeloma. The dose will vary depending on the disease being treated. Therapeutic doses will be administered in sufficient amounts to reduce pain, inhibit tumor growth, cause regression of tumors, and/or kill the tumor. The amount of radionuclide needed to provide the desired therapeutic dose may be determined experimentally and optimized for each particular composition. The amount of radioactivity required to deliver a therapeutic dose may vary with the individual composition employed. The dosage to be administered may be given in a single treatment or fractionated into several portions and administered at different times.

As used herein, “pharmaceutically acceptable salt” means any salt of a compound of formula (I) which is sufficiently non-toxic to be useful in therapy or diagnosis of mammals. Thus, the salts are useful in accordance with this invention. Representative of those salts, which are formed by standard reactions, from both organic and inorganic sources include, for example, sulfuric, hydrochloric, phosphoric, acetic, succinic, citric, lactic, maleic, fumaric, palmitic, cholic, palmoic, mucic, glutamic, d-camphoric, glutaric, glycolic, phthalic, tartaric, formic, lauric, steric, salicylic, methanesulfonic, benzenesulfonic, sorbic, picric, benzoic, cinnamic acids and other suitable acids. Also included are salts formed by standard reactions from both organic and inorganic sources such as ammonium, alkali metal ions, alkaline earth metal ions, and other similar ions. Particularly preferred are the salts of the compounds of formula (I) where the salt is potassium, sodium, ammonium, or mixtures thereof.

### EXAMPLES

### Example 1

Rapid and Specific Targeting of Radiolabeled Neutrokine-Alpha to Lymphoid Tissues

Here, biodistribution studies of radiolabeled Neutrokine-alpha are reported that demonstrate high in vivo targeting specificity of Neutrokine-alpha for lymphoid tissues. Neutrokine-alpha was radiolabeled with ¹²⁵I and injected intravenously into BALB/c mice. Three doses and 4 timepoints over a 24-hr period were studied. Biodistribution was measured by direct counting of the radioactivity in dissected whole organs or tissues and by whole body quantitative autoradiography (QAR).

Spleen and lymph nodes showed the highest concentration of radioactivity among the dissected organs and tissues. Three hours after injection of 0.01 mg/kg Neutrokine-alpha, 63% and 23% injected dose (ID)/g were measured in spleen and lymph node, respectively, compared to ˜5% for both kidney and liver. As the dose was increased to 0.05 mg/kg or 0.3mg/kg, the %ID/g in spleen and lymph node decreased but was unchanged in liver and kidney, suggesting that targeting to spleen and lymph nodes is mediated by saturable binding. With increasing time, the ratio of the concentration in spleen and lymph node to the concentration in either kidney or liver increased. QAR confirmed the high uptake of radiolabeled Neutrokine-alpha in spleen and lymph nodes at 3 hr, and revealed high uptake in bone marrow, gut-associated lymphoid tissue (GALT) and intestinal contents as well. At 24 hr, spleen, lymph nodes and GALT were still strongly positive for radiolabeled Neutrokine-alpha by QAR whereas liver and kidney no longer had observable levels. A cytotoxic radionuclide coupled to Neutrokine-alpha could irradiate neoplastic B-cells trafficking through or residing in lymphoid tissues. Thus, the rapid and highly specific targeting of radiolabeled Neutrokine-alpha to lymphoid tissues provides a rationale for its application in the treatment of B-cell malignancies.

A similar biodistribution analysis was performed with Neutrokine-alpha attached chelator according to Formula I labeled with ¹¹¹In (NA-MeO-DOTA-¹¹¹In) . Female BALB/c mice weighing approximately 20 g were used to determine the biodistribution of NA-MeO-DOTA-¹¹¹In. Mice were injected intravenously via the tail vein with 0.1 mL/g at 5 μg/mL NA-MeO-DOTA-¹¹¹In to give a protein dose of 50 μg/kg. The specific activity of the NA-MeO-DOTA-¹¹¹In was 13.6 μCi/μg, resulting in approximately 13.6 μCi per injected animal. Prior to injection, 84% of the radioactivity in the dosing solution was determined to be TCA-precipitable (protein-associated). Biodistribution was determined by both tissue dissection and quantitative whole body autoradiography (QWBA). Timepoints of 3, 6, 24 72 and 96 hours post injection were studied. The following tissues were dissected and studied: spleen, mesenteric lymph node, kidney, part of the liver, stomach, part of the duodenum, heart, lung, thymus (72 and 96 hours only) skeletal muscle (biceps femoris) and part of the femur.

Similar to the biodistribution of ¹²⁵I labeled Neutrokine-alpha, the highest % injected dose (%ID) were found in the spleen and mesenteric lymph nodes (C max=63.4 and 24.6%ID/g, respectively). This localization agrees with the expression of the three known Neutrokine-alpha receptors (BCMA, TACI, and BAFF-R) found predominantly on mature, immunoglobulin-positive B cells. The activity observed in the femur Cmax=8.4%ID/g is believed to be due to the localization of NA-MeO-DOTA-¹¹¹In to mature B cells in the bone marrow and the activity in the duodenum (Cmax=7.2%ID/g) may represent localization to B cells in the lamina propria of the gut. Significant radioactivity was also found in the liver (Cmax=19%ID/g). The kidneys, stomach, heart, lung, muscle and thymus all had relatively low %ID/g. The high radioactivity seen in the liver is believed to be due to retention of a metabolic end product, ¹¹¹In-chelator-lysine. Because of its ionic character at intralysosomal pH, ¹¹¹In-chelator-lysine becomes trapped intracellularly at the site of metabolism. In contrast, ¹²⁵I labeled Neutrokine-alpha studies did not reveal high levels of radioactivity in liver because the free ¹²⁵I released by the intracellular metabolism of halogenated proteins can rapidly diffuse across cell membranes and is not retained at the site of metabolism.

### Example 2

Pharmacological Effects of ¹³¹I-Labeled Neutrokine-Alpha in BCL1 Tumor-Bearing Mice and J558 Tumor Bearing Mice

¹³¹I-Neutrokine-Alpha Administration to BCL1 Tumor-Bearing Mice

The BCL1 tumor cell line was derived from a spontaneous murine B cell tumor. Intraperitoneal inoculation of the BCL1 cell line in BALB/c mice results in splenomegaly, and subsequent death. The BCL1 tumor cell phenotype is IgM positive, complement receptor negative, Fc receptor positive and has marginal IgD expression (Knapp et al., J. Immunol. 123:992-999 (1979) and Vitetta et al. Blood 89:4425-36. (1997)). Based on FACS analysis using biotinylated Neutrokine-alpha, BCL1 cells freshly isolated from the spleens of BALB/c mice express Neutrokine-alpha receptors on their cell surface. The BCL1 tumor model is a relevant mouse model for human B cell lymphoma, providing a means to test the ability of ³¹I-labeled Neutrokine-alpha to kill leukemic B cells and consequently prolong survival of tumor-bearing mice. Three lots of ¹³¹I-labeled Neutrokine-alpha (Lots TX1, TX2 and TX3) were prepared by MDS Nordion (Ontario, Canada) and used in 3 different experiments to evaluate the effects of ¹³¹I-labeled Neutrokine-alpha in this murine model.

Female BALB/c mice were injected intraperitoneally (ip) on Day 0 with 1×10⁵ viable BCL1 cells that had been propagated in vivo. Treatment groups for the 3 experiments are described in Table VI. Ten days after injection of tumor cells, the animals were administered ¹³¹I-labeled Neutrokine-alpha iv in 110 μL. The doses administered were 11.9 or 15.3 mCi/kg (TX1), 17.5 mCi/kg (TX2), or 37.7 mCi/kg (TX3) for the 3 experiments. To identify potentially toxic effects of the administered 131I-labeled Neutrokine-alpha, age-matched control BALB/c mice without BCL1 tumors were injected with identical doses of the ¹³¹I-labeled protein. An additional group of BALB/c mice, bearing BCL1 tumors and receiving an iv injection of the vehicle, served as the normal tumor control group. Survival was then monitored for 48, 44, or 40 days for the TX1, TX2, and TX3 experiments, respectively.

TABLE VI
Treatment groups for TX1, TX2 and TX3 experiments

| | | | | BCL1 |
|---|---|---|---|---|
| | | ¹³¹I-Neutrokine- | | Tumor |
| | | alpha Dose | | Inoculated ip |
| Exp. | Group | (mCi/kg) | n | (No. of cells) |
| 1 (TX1) | 1 Vehicle | 0 | 15 | 1 × 10⁵ |
| | 2 ¹³¹I-Neutrokine-alpha | 11.9 | 10 | 1 × 10⁵ |
| | 3 ¹³¹I-Neutrokine-alpha | 15.3 | 10 | 1 × 10⁵ |
| | 4 ¹³¹I-Neutrokine-alpha | 11.9 | 10 | 0 |
| | 5 ¹³¹I-Neutrokine-alpha | 15.3 | 10 | 0 |
| 2 (TX2) | 1 Vehicle | 0 | 12 | 1 × 10⁵ |
| | 2 ¹³¹I-Neutrokine-alpha | 17.5 | 12 | 1 × 10⁵ |
| | 3 ¹³¹I-Neutrokine-alpha | 17.5 | 8 | 0 |
| 3 (TX3) | 1 Vehicle | 0 | 14 | 1 × 10⁵ |
| | 2 ¹³¹I-Neutrokine-alpha | 37.7 | 14 | 1 × 10⁵ |
| | 3 ¹³¹I-Neutrokine-alpha | 37.7 | 8 | 0 |

The endpoint monitored in the 3 experiments was survival (days) following ip inoculation of BCL1 tumor cells. All animals were examined daily. The day post-inoculation that mice were either found dead or in moribund condition (the latter being immediately euthanized for humane reasons) was recorded.

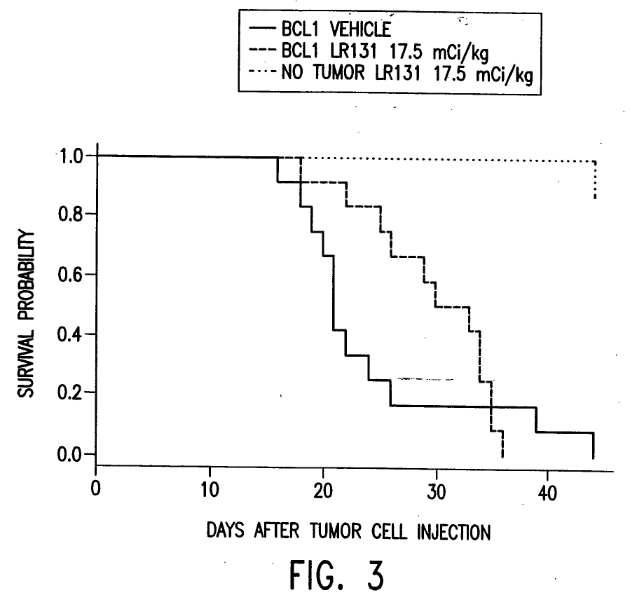

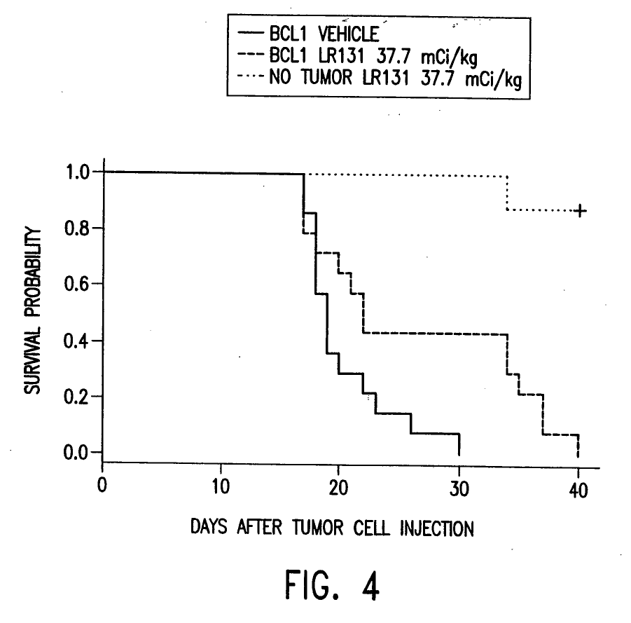

A single iv administration of either 11.9 or 15.3 mCi/kg (TX1), 17.5 mCi/kg (TX2), or 37.7 mCi/kg (TX3) of ¹³¹1-labeled Neutrokine-alpha injected 10 days after intraperitoneal inoculation of BCL1 cells in BALB/c mice significantly improved survival compared with mice inoculated with tumor and treated with the ¹³¹I-labeled Neutrokine-alpha vehicle (FIGS. 2-4;

in FIGS. 2-4, ¹³¹I-labeled Neutrokine-alpha is indicated as LR131). The median survival time for the vehicle-treated, tumor-bearing mice was 18, 21, and 19 days post-tumor cell injection for the TX1, TX2, and TX3 experiments, respectively. In the TX1 experiment, ¹³¹1-labeled Neutrokine-alpha administration at dose levels of 11.9 and 15.3 mCi/kg doubled the median survival time of tumor-bearing mice to 35.5 (11.9 mCi/kg) and 34 (15.3 mCi/kg) days post-treatment, respectively. In the TX2 and TX3 experiments, ¹³¹I-labeled Neutrokine-alpha administration at a dose of 17.5 or 37.7 mCi/kg increased the median survival time of tumor-bearing mice to 30 and 22 days post-treatment, respectively. Tumor-bearing mice treated with all doses of ¹³¹I-labeled Neutrokine-alpha in the 3 experiments had a significantly lower risk of dying than tumor-bearing mice treated with vehicle (Table VII).

TABLE VII
Incidence of mortality for TX1-TX3 experiments

| | | Median |
|---|---|---|
| Ex- | | Survival |
| peri- | | Time |
| ment | Treatment Group | (Days) |
| TX1 | 1, BCL1 + ¹³¹I-labeled Neutrokine-alpha (11.9 mCi/kg) | 35.5 |
| | 2, BCL1 + ¹³¹I-labeled Neutrokine-alpha (15.3 mCi/kg) | 34 |
| | 3, BCL1 Tumor Only | 18 |
| | 4, No Tumor + ¹³¹I-labeled Neutrokine-alpha | >48 |
| | (11.9 mCi/kg) | |
| | 5, No Tumor + ¹³¹I-labeled Neutrokine-alpha | >48 |
| | (15.3 mCi/kg) | |
| TX2 | 1, BCL1 + ¹³¹I-labeled Neutrokine-alpha (17.5 mCi/kg) | 30 |
| | 2, BCL1 Tumor Only + vehicle | 21 |
| | 3, No Tumor + ¹³¹I-labeled Neutrokine-alpha | >44 |
| | (17.5 mCi/kg) | |
| TX3 | 1, BCL1 + vehicle | 19 |
| | 2, BCL1 + ¹³¹I-labeled Neutrokine-alpha (37.7 mCi/kg) | 22 |
| | 3, No tumor + ¹³¹I-labeled Neutrokine-alpha | >40 |
| | (37.7 mCi/kg) | |

In the TX1-TX3 series of experiments, the effect that increasing the dose of ¹³¹I-labeled Neutrokine-alpha had on the survival of the BCL1 tumor-bearing animals was investigated. A maximal survival benefit was achieved with the low doses of ¹³¹I-labeled Neutrokine-alpha (11.9 and 15.3 mCi/kg). The much reduced effectiveness of ¹³¹I-labeled Neutrokine-alpha in TX3 may be due to toxicity associated with the high dose of the material used.

In conclusion, a single iv administration of ¹³¹I-labeled Neutrokine-alpha administered to mice bearing BCL1 leukemia cell splenic tumors significantly improved survival compared with tumor-bearing mice treated with vehicle.

¹³¹I-Neutrokine-Alpha Administration to J558 Tumor-Bearing Mice

In a similar experiment as that described above, BALB/c mice were injected subcutaneously with J558 plasmacytoma cells (ATCC # TIB-6) and treated with a single intravenous treatment of 25mCi/kg of ¹³¹I-labeled Neutrokine-alpha. 24 BALB/c mice (NCI, 4 weeks old, average weight 18 g) were divided into 2 groups (12 mice per group) and injected sc with 2.5×10⁵ J558 cells in 100 mL of PBS. At Day 9 after injection, mice in Group 1 were injected intravenously with 100 mL of formulation buffer, and mice in Group 2 were injected iv with a dose of 25 mCi/kg of ¹³¹I-Neutrokine-alpha in 100 mL of formulation buffer. The average body weight at the time of ¹³¹I-Neutrokine-alpha injection was 19.5 g.

Two parameters were evaluated during this study the tumor size and the time to tumor response. To evaluate tumor size the short and long axes of the tumor were measured using an electronic digital caliper. Tumor size was calculated by multiplication of the lengths of the short and long axes and expressed in mm². The time to tumor response was characterized by the day after cell inoculation when a visible tumor (>2 mm) was detected on a mouse. In addition, mice were monitored for survival and signs of radiation induced toxicity (general appearance, activity, breathing frequency, stool consistence).

One mouse in the ¹³¹I-Neutrokine-alpha-treated group died on Day 25 (16 days after ¹³¹I-Neutrokine-alpha treatment) with no obvious signs of radiation related toxicity. A second mouse died in the same group on Day 30, when all animals in the control group were terminated because of large tumor size.

The first tumors of measurable size were detected at Day 14 in the buffer control group, where 4 out of 12 animals developed tumors. In the ¹³¹I-Neutrokine-alpha treated animals, tumor formation was delayed by 6 days. Only one mouse out of 12 developed a tumor at Day 20. At Day 22, there was only one tumor-bearing mouse in the ¹³¹I-Neutrokine-alpha treated group out of 12 animals, whereas in the buffer control group, 11 out of 12 mice developed tumors of different sizes. At Day 27, the mean tumor size in the buffer control group was 489 mm² (all tumor positive mice in this group were terminated at this time point). In the ¹³¹I-Neutrokine-alpha treated group, the mean tumor size was 32.7 mm2, 15 times smaller than in the buffer control group. Taken together, these data suggest a strong inhibition of J558 tumor development in mice treated with ¹³¹1-Neutrokine-alpha at a dose of 25 mCi/kg and tumor load of 2.5×10⁵ cells/mouse.

In conclusion, a single intravenous administration of ¹³¹I-Neutrokine-alpha into BALB/c mice at a dose of 25 mCi/kg significantly inhibits subcutaneous growth of J558 plasma cell tumors. At the initial tumor load of 2.5×10⁵ cells/mouse, a 6 day delay in tumor formation and a 15-fold reduction in tumor size was observed in ¹³¹I-Neutrokine-alpha treated animals.

The anti-neoplastic effects of ¹³¹I-Neutrokine-alpha were accompanied by the expected B lymphocyte hypoplasia and a transient (<20 days) depletion of cKit⁺ bone marrow precursors and peripheral platelets. Peripheral neutrophil, red blood cell, and monocyte counts were unaffected by ¹³¹I-Neutrokine-alpha treatment. Taken together, the results demonstrate that ¹³¹I-Neutrokine-alpha inhibits in vivo tumor growth in two models of B cell neoplasia. Moreover, ¹³¹I-Neutrokine-alpha efficacy was not accompanied by significant bone marrow toxicities or peripheral myelosuppression.

### Example 3

Method For Producing Neutrokine-Alpha Using a Stringent Promoter and Low Expression Level

Neutrokine-alpha has been produced in Escherichia coli K-12 from the periplasmic fraction of the cell lysate. Using this system, soluble, properly folded, active Neutrokine-alpha is not obtainable from simple shake flask experiments. Yields of soluble Neutrokine-alpha from complex media fermentations in small and large-scale bioreactors are on the order of 1-5 mg/L. Greater yields (25-38 mg/L) of soluble, properly folded, active Neutrokine-alpha can be accomplished in bioreactors at low to medium cell density under defined medium conditions. Moreover, this low quantity of protein is difficult to purify via conventional methods.

This example describes a method for the production of high yields of soluble, properly folded, active Neutrokine-alpha in the periplasm of Escherichia coli, which permits the use of conventional methods for Neutrokine-alpha purification, such as those described below. Additionally, Neutrokine-alpha protein may be purified using affinity columns comprising Neutrokine-alpha binding peptides such as those described in WO 02/02641, which is herein incorporated by reference in its entirety. Purified Neutrokine-alpha may be quantified using RP-HPLC.

This method relies on the expression of Neutrokine-alpha protein from the bacterial phoA promoter. The phoA promoter is a very tightly regulated system that exhibits a very low level of transcription in the presence of excess phosphate. As the phosphate level in the medium decreases below a threshold of 4 micromolar (Wanner, B. L., J. Cell Biochem 51:47 (1993)), transcription is induced about 1000-fold. The phoA promoter yields a gradual build-up of recombinant protein, instead of a sharp increase of induction that occurs with other systems. This gradual or steady increase in recombinant protein minimizes the chance of overwhelming the components of the bacterial expression system and may also minimize the formation of inclusion bodies. Furthermore, this gradual build up permits the expression of proteins that might have been toxic to the cell if they were induced to high levels over a short period of time.

Expression Vector pML124

The expression vector, pML124, was created using pBR322 as the starting backbone. First, the endogenous NdeI site of pBR322 was eliminated by digesting it with NdeI, filling in the overhanging ends with the Klenow enzyme, then re-ligating the two blunt-ends back together (this created pML123). Next, pML123 was digested with EcoRI and BamHI restriction enzymes and the linear plasmid (loss of ˜375 bp of DNA) was agarose gel purified (Qiagen).

The phoA promoter region was PCR-amplified from the E. coli K-12 chromosome (W3110; ATCC Catalogue No. 27325) with EcoRI (5′) and BamHI (3′) engineered sites. NdeI and KpnI sites were also engineered downstream of the phoA promoter to facilitate cloning of recombinant genes. Finally, the Shine-Dalgamo (SD) box was optimized for protein expression. The wild-type SD box and its adjacent sequence is as follows (the putative SD boxes are underlined and in bold):

5′-TTTGTACATGGAGAAAATAAA (SEQ ID NO:12):-[ATG, start of coding sequence]-3′

Optimized SD box and adjacent sequence is as follows:

5′-CACGTAAAGGAAGTATCTCAT (SEQ ID NO:13)-[ATG, start of coding sequence]-3′

The digested (EcoRI and BamHI) and purified phoA promoter PCR product was ligated into the agarose gel purified pML123 (described above). The ligation mixture was transformed into highly competent E. coli cells using standard techniques. Positive clones were identified via restriction analysis and DNA sequencing.

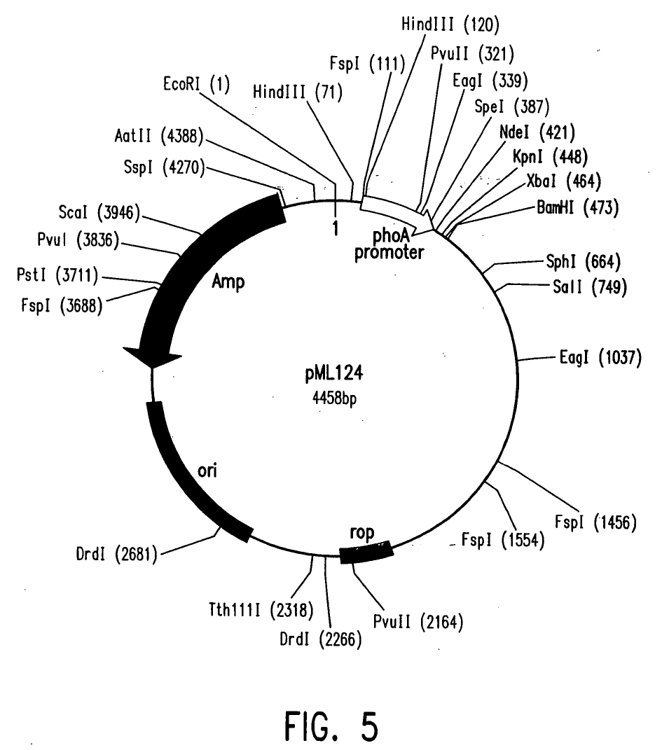

pML124 contains a gene for ampicillin resistance, a ColE1 replicon (pBR322-based), Rop, phoA promoter, the optimized Shine-Dalgarno (SD) box (above) and a multiple cloning site. FIG. 5 is a plasmid map of pML124 and SEQ ID NO:14 is the nucleotide sequence of pML124. Additionally, plasmid pML124 was deposited at the American Type Culture Collection (ATCC) on Oct. 8, 2001 and given ATCC Deposit No. PTA-3778. ATCC Deposit Nos. PTA-3778 was made pursuant to the terms of the Budapest Treaty on the International Recognition of the Deposit of Microorganisms for the Purposes of Patent Procedure. The ATCC (American Type Culture Collection) is located at 10801 University Boulevard, Manassas, Va. 20110-2209.

Neutrokine-Alpha Expression Vector pML124-MBPssBLyS

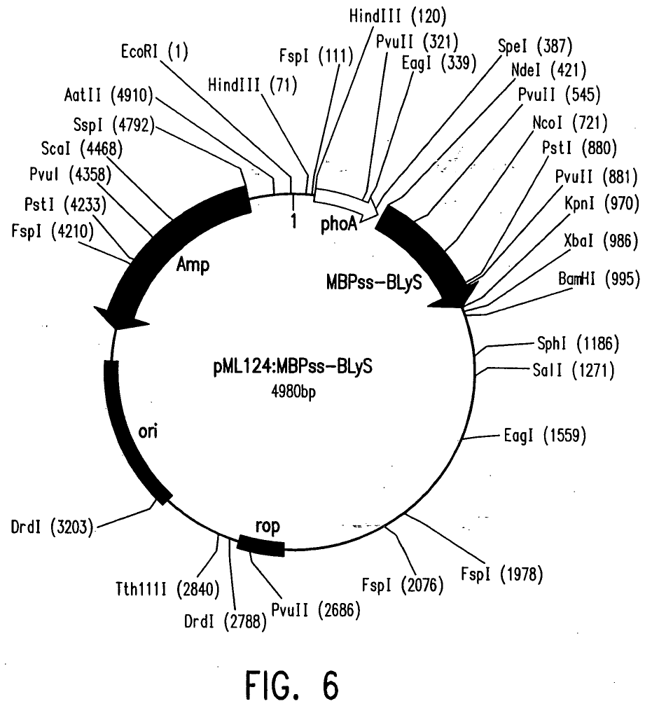

A fusion construct of the maltose binding protein signal sequence (MBPss) and Neutrokine-alpha was placed behind the phoA promoter in pML124 as follows. A 549 bp NdeI/KpnI MBPss-Neutrokine-alpha containing DNA insert was ligated into NdeI/KpnI digested and gel purified pML124 to form pML124-MBPss-BLyS. (FIG. 6, SEQ ID NO:15 ATCC Deposit No. PTA-3867, deposited Nov. 16, 2001). ATCC Deposit No. PTA-3867 was made pursuant to the terms of the Budapest Treaty on the International Recognition of the Deposit of Microorganisms for the Purposes of Patent Procedure. The ATCC (American Type Culture Collection) is located at 10801 University Boulevard, Manassas, Va. 20110-2209.

The pML124 plasmid (FIG. 5, SEQ ID NO:14) is described in above and in U.S. Provisional Applications 60/329,508 filed Oct. 17, 2001, 60/329,747 filed Oct. 18, 2001 and 60/331,478 filed Nov. 16, 2001 which are herein incorporated by reference in their entireties. The phoA promoter region is located at nucleotides 111410 SEQ ID NOs:14 and 15. The MBP signal sequence is encoded by nucleotides 423-500 of SEQ ID NO:15 and nucleotides 501-959 of SEQ ID NO:15 encode amino acids 134-285 of Neutrokine-alpha (SEQ ID NO:2). The amino acid sequence of the MBP signal sequence is shown in SEQ ID NO:16 and the amino acid sequence of the full length MBP signal sequence-Neutrokine-alpha protein encoded by the pML124-MBPss-BLyS vector is shown in SEQ ID NO: 17.

Neutrokine-Alpha Expression in E. coli

Plasmid pML124-MBPss-BLyS was transformed into E. coli cells, e.g. K-12 based strains, by standard methods. Ampicillin resistant transformants were screened for the proper DNA insert by restriction enzyme analysis and DNA sequence. For example, digestion of pML124-MBPss-BLyS™ with NdeI and KpnI results in two nucleotide fragments: 549 and 4,431 base pairs in length. Positive clones were subsequently grown in City Broth-Low Phosphate media (see recipe below). Neutrokine-alpha expression levels were examined via SDS-PAGE and subsequent Coomassie staining. Using simple shake flask experiments, more than 260 mg/L of Neutrokine-alpha was obtained.

Next, positive clones were grown to high cell density in complex media in small scale bioreactors, similar to the method described by Joly et al., PNAS 95:2773-2777 (1998), which is hereby incorporated by reference in its entirety. Specifically, the initial fermentation medium for the 5 L bioreactor was composed of 55.7 mM ammonium sulfate, 13.9 mM sodium monobasic phosphate, 21.9 mM potassium dibasic phosphate, 5 mM sodium citrate, 29.6 mM potassium chloride, 14.7 mM magnesium sulfate, 1.11% NZ-amine AS, 1.11% yeast extract, 5 g/L glucose, 0.002% ferric chloride, 25 μg/ml kanamycin. A trace element solution (2.5 ml/3.4 L) was added containing 100 mM ferric chloride plus 30 mM of the following components: zinc sulfate, cobalt chloride, sodium molybdate, copper sulfate, boric acid and manganese sulfate. The fermenter was operated at 30° C., 650 rpm agitation, 10 standard liter/minute aeration. When the initial glucose was depleted, a concentrated glucose solution (50%) was added until the dissolved oxygen (DO) concentration reached 20% of air saturation as measured by an on-line oxygen electrode. When the optical density (600 nm) reached 40 OD600, a solution of 20% NZ amine AS, 20% yeast extract was fed at 0.2 ml/min for the rest of the fermentation. Neutrokine-alpha production was on the order of 260-570 mg/L.

Low Phosphate Containing Media:

City Broth-Low Phosphate

30 mM (NH₄)₂SO₄; 2.25 mM NaCitrate-2H₂O; 12 mM MgSO₄; 15 mM KCl; 5% Yeast extract; 2% Casamino acids; 110 mM MOPS; 33 mM Glucose; pH 7.3

Vegan City Broth-Low Phosphate

30 mM (NH₄)₂SO₄; 2.25 mM NaCitrate-2H₂O; 12 mM MgSO₄; 15 mM KCl; 5% Phytone; 2% Casamino acids; 110 mM MOPS; 33 mM Glucose; pH 7.3.

The only difference between the two media is that Phytone is substituted for Yeast extract in the Vegan recipe.

Purification of Neutrokine-Alpha

10 grams of E. coli cell paste are suspended in 50 milliliters of 5 mM sodium citrate, pH 6.0 and placed at 4° C. for 1 hour with gentle shaking. Cells are then disrupted by passing them through an M-Y110 Microfluidizer® Processor (Microfluidics, Inc., Newton, Mass.) set at 7500 psi four times. The suspension is then centrifuged at 22,000×g for twenty minutes at 4° C. using a Sorvall SLA-1500 rotor. The supernatant is then collected and filtered through a 0.45 micron bottle top filter (Nalgene).

Filtered supernatant is then loaded at 9 centimeters/hour on a Fast Flow Sepharose DEAE column (Amersham Bisociences, Piscataway, N.J.) previously equilibrated with 5 mM sodium citrate, pH6.0 (equilibration buffer). After loading, the column is washed with 5 to 10 column volumes of equilibration buffer. The Neutrokine-alpha protein is eluted with a 200 mM NaCl step in equilibration buffer. Buffers used with the Fast Flow Sepharose DEAE chromatography column are pre-filtered using a 0.22 micron CA bottle top filter (Nalgene) and pre-chilled to 4° C. The Fast Flow Sepharose DEAE column is used at 4° C. Prior to use, columns are cleaned with 0.5 M NaOH.

Relevant fractions, as determined by the ratio of contaminating proteins to Neutrokine-alpha protein seen in Coomassie stained SDS-PAGE gels, are pooled and diluted 1:1 with lOmM sodium citrate, pH 6.0, 2M (NH₄)SO₄. Pooled fractions are loaded at 17 centimeters/hour onto a Polypropylene Glycol Hydrophobic Interaction chromatography column (Tosoh Biosep, Montgomeryville, Pa.) previously equilibrated with 10 nM sodium citrate, pH 6.0, 1M (NH₄)SO₄ (loading buffer). After loading, the column is washed with 5-10 volumes of loading buffer. The Neutrokine-alpha protein is eluted with a 5 column volume gradient from loading buffer to elution buffer (10 mM sodium citrate, pH 6.0). Neutrokine-alpha elutes in the second peak toward the end of the gradient absorbance at 280 nm. Buffers used with the Polypropylene Glycol Hydrophobic Interaction chromatography column are pre-filtered using a 0.22 micron CA bottle top filter (Nalgene) and used at room temperature. The Polypropylene Glycol Hydrophobic Interaction chromatography column is also used at room temperature. Prior to use, columns are cleaned with 0.5 M NaOH.

Relevant fractions, determined by the ratio of contaminating proteins to Neutrokine-alpha protein as monitored by Coomassie stained SDS-PAGE gels, are pooled and are dialyzed overnight (12 hours) into 50 mM Tris, pH 7.4, 50 mM NaCl at 4° C. The dialyzed pool is then loaded onto a POROS PI-50 anion exchange chromatography column (Applied Biosystems, Foster City, Calif.), previously equilibrated with 50 mM Tris, pH 7.4, 50 mM NaCl, at 17 centimeters/hour. After loading, column is washed with 5-10 volumes of loading buffer. Neutrokine-alpha is eluted using a pH step from 50 mM Tris, pH 7.4, 50 mM NaCl buffer to 50 mM sodium citrate, pH 6.0. Relevant fractions, as determined by the ratio of contaminating proteins to Neutrokine-alpha protein seen in Coomassie stained SDS-PAGE gels, are pooled and stored at 4° C. Buffers used with the POROS PI-50 anion exchange chromatography column are pre-filtered using a 0.22 micron CA bottle top filter (Nalgene) and pre-chilled to 4° C. The POROS PI-50 anion exchange chromatography column is used at 4° C. Prior to use, columns are cleaned with 0.5 M NaOH.

This purification protocol yields 0.5-1 milligram per gram of starting cell paste based on BCA protein assay (Pierce Biotechnology, Rockford, Ill.) and absorbance at 280 nanometers. The protein is 96% pure as determined by reverse phase-high performance liquid chromatography (RP-HPLC). Native-PAGE and size exclusion chromatography-HPLC (SEC-HPLC) analysis indicates the protein is predominantly in trimeric form.

The production of MBPss-Neutrokine-alpha under control of the phoA promoter allowed more stringent, slower expression, and resulted in increased yields. In summary, the production of Neutrokine-alpha from the phoA system is scaleable and achieves 10 to 20-fold more soluble, properly folded, active material than the current system.

Alternatively, Neutrokine-alpha can be purified by any method known in the art. In a non limiting example, Neutrokine-alpha proteins may purified by affinity chromatography using columns to which Neutrokine-alpha binding peptides are attached. Neutrokine-alpha binding peptides that may be used in affinity purification of Neutrokine alpha are described for example, in WO2002/16411 and WO2002/16412 which are herein incorporated by reference in their entireties).

### Example 4

### Preparation of Neutrokine-Alpha-DOTA Conjugate

The following materials are used in the preparation of the NA-chelator conjugate:

Neutrokine-alpha protein (in citrate buffer (see below);

concentration of about 2.4 mg/mL);

MeO-DOTA-NCS;

HEPES buffer (about 500 mM and about pH 8.5);

citrate buffer (10 mM sodium citrate, 140 mM NaCl, pH 6.0);

1 M Glycine-HCl (Glycine buffer);

1 N NaOH solution;

water for injection (WFI);

diafiltration buffer (10 mM sodium acetate, 140 mM NaCl, pH 6.0);

Millipore Pellicon XL regenerated cellulose membrane −10 kD molecular weight cut off (MWCO).

Reaction vessels for preparing Neutrokine-alpha-DOTA conjugates and Neutrokine-alpha-DOTA complexes of the invention are preferably disposable plastic containers to minimize contamination with heavy metal ions

Into a sterile container (reaction vessel) was placed 708.3 mL of Neutrokine-alpha protein solution (concentration about 2.4 mg/mL in citrate buffer). The Neutrokine-alpha protein is a homotrimer of three molecules of Neutrokine-alpha each having a sequence of amino acids 134-285 of SEQ ID NO:2. Citrate buffer, 311.2 mL, was added into the sterile container and gently mixed. 60 μL of the diluted Neutrokine-alpha protein was mixed with 40 μL of citrate buffer and set aside in a 1.5 mL Eppendorf tube labeled “Control”. Freeze down sample or keep it on ice for a short period of time and then freeze.

About 153 mL of HEPES buffer were added to the reaction vessel and gently swirled to mix.

The MeO-DOTA-NCS solution is prepared in a separate container. MeO-DOTA-NCS, 11.9 g, was placed into a separate container to which 170 mL of water for Injection and 34 mL IN NaOH solution were added. The container was mixed by inversion until all MeO-DOTA-NCS was dissolved.

The quantity MeO-DOTA-NCS used is established by the approximate 1:11 molar ratio of chelator bonding sites in Neutrokine-alpha to MeO-DOTA-NCS molecules optimal for giving one MeO-DOTA moiety, on average, per Neutrokine-alpha monomer (or 3 MeO-DOTA moieties per Neutrokine-alpha trimer). In this case, the Neutrokine-alpha protein contains 11 chelator bonding sites (Ala-134, Lys-160, Lys-173, Lys-181, Lys-184, Lys-188, Lys-204, Lys-215, Lys-216, Lys-252, and Lys-283).

The MeO-DOTA-NCS solution was then added to the reaction vessel containing the Neutrokine-alpha protein. The reaction vessel was then gently mixed. The container that contained the MeO-DOTA-NCS solution was then rinsed with about 153 mL of water for injection, which was then added to the reaction vessel.

If pH of the solution in the reaction vessel was not at pH 8.5±0.1, the pH was adjusted with 1 M NaOH or 1 M citric acid as necessary. The volume of titration necessary to adjust pH was recorded.

The reaction vessel was placed into a water bath and was gently agitated for 4.5 to 5.5 hours at about 25° C. At the end of incubation time, 68 mL of glycine buffer was added to the reaction vessel to stop the reaction. The reaction was incubated for approximately an additional 15 minutes at about 25° C. The volume of the reaction solution was adjusted to about 1700 mL by addition of citrate buffer (10 MM sodium citrate, 140 mM sodium chloride, pH 6.0). One sample (0.5 mL) of the reaction solution was obtained and labeled as “prediafiltration sample.” The prediafiltration sample was lyophilized or kept on ice.

### Example 5

### Diafiltration Procedure

One 0.1 m² Pellicon XL™ regenerate cellulose membrane cartridge (Millipore) was prepared according to manufacturer's instruction manual included with the cartridge. The flow rate of the peristaltic pump is set to about 400 mL/min. The membrane was sanitized with 0.1 N NaOH for at least about 30 minutes. At least about 1.0 L of diafiltration buffer was flushed through the retentate port, and at least about 1.0 L diafiltration buffer was flushed through the permeate port to equilibrate system. Neutrokine-alpha conjugate was concentrated approximately 2.5-fold.

Maintaining the volume within the reaction volume, ten diafiltration volumes (DV) of buffer were exchanged.

A sample of the solution, about 0.5 mL, was obtained and labeled as “post diafiltration (DF)” sample. The post DF sample was lyophilized or kept on ice. The membrane was cleaned according to manufacturer's instructions.

Samples were analyzed by reverse phase HPLC for purity and by mass spectrometry to estimate the amount of chelator molecules covalently linked to each Neutrokine-alpha monomer. A Bradford assay was used to determine the Neutrokine-alpha conjugate concentration.

### Example 6

### Incorporation Of ⁹⁰Y Into Neutrokine-Alpha Conjugate

The Neutrokine-alpha conjugate, as prepared in Example 5, was used to prepare a Neutrokine-alpha complex comprising ⁹⁰Y. The ratio of chelator to protein in the conjugate was about 1.13. 50 μL of Neutrokine-alpha conjugate (1 mg/mL in an acetate buffer (0.1 M, pH 6.0)) was placed in a vial. About 2 μL of the following yttrium solution was added: yttrium solution consisted essentially of 19 μL of 15 mM Y³⁺ in 1 mM HCl and 1 μL (10 μCi) of ⁹⁰Y³⁺ in 1 mM HCl. The reaction solution was mixed and allowed to stand for about 30 minutes at room temperature (about 23° C.). 2 μL of 23.6 mM DPTA was added. Reaction mixture was allowed to stand for about 5 minutes.

Chelation efficiency may be determined using an instantaneous thin layer chromatography (ITLC) kit which is commercially available from BioDex Medical, Shirley, N.Y. (Tec-control Kit #151-770). In ITLC, radionuclide not associated with protein will migrate with the solvent front whereas radionuclide associated with a Neutrokine-alpha complex will migrate more slowly. Comparison of the amount of the radioactivity in the dye front compared to the amount of radioactivity in the portions of the ITLC strip closer to the sample will give the chelation efficiency. Briefly, the final drug product is diluted 1 to 10 in saline (0.9%). 2 microliters of the diluted product is deposited on the ITLC strip. The strip is developed in saline (1 milliliter). The strip is dried and cut in two halves; each half is deposited in a glass tube. The free metal is measured by the Gamma Well counter (Packard Instruments Model 5003) and reported as a percentage of the total activity.

The above procedure was repeated varying the time of the chelating reaction (0, 5, 10, 20, and 30 minutes before adding EDTA or DPTA) and varying the concentration of the yttrium (2× concentration).

### Example 7

### Preparation of Neutrokine-Alpha Complex for Therapeutic Use

A Neutrokine-alpha complex is prepared by reacting a Neutrokine-alpha conjugate with ⁹⁰YCl₃. 1.0 (2 mg/ml) mL of a first solution comprising the Neutrokine-alpha conjugate as prepared in Example 5 (2 mg/mL) is added to a reaction vial containing 1.0 mL of acetate buffer (275 mM, pH8.4). 1.0 mL of a solution of ⁹⁰YCI3 (60 mCi/mL) is added to the reaction vial. The reaction vial is allowed to stand or gently mixed or agitated for up to 45, preferably 30-45 minutes, more preferably 30 minutes. After the given amount of time, 7.0 mL of a solution comprising an acetate buffer (10 mM), NaCl (140 mM) ascorbate (4.0%) and DTPA (1 mM) at pH 6.0 is added to the reaction vial. The labeling reaction can be optimized using routine techniques that are known in the art. For example, by varying the pH, reactant concentrations (neutrokine-alpha conjugate, metal ion), reaction vessel material, and temperature, percent incorporation of the metal ion into a complex can be maximized and reaction time minimized.

⁹⁰Y Labeling Protocol

In an 10 cc vial from West (unwashed) combine:

1.8 milliliters of sodium acetate (NaOAc, 275 mM) with 0.4 milliliters of sodium bicarbonate (NaHCO₃, 377 mM).

70 mCi of sterile ⁹⁰Y (MDS Nordion, Ontario, Canada)

1 milliliter of Neutrokine-alpha conjugate as prepared in Examples 4-5 (2 mg/mL).

Mix the vial by gentle inversion and react for 45 minutes at room temperature. Stop the reaction by adding diluent (140 mM NaCl, 2 mM DPTA, and 10% sodium ascorbate) to a final volume of 9.95 milliliters. Let the mixture stand for five minutes prior to proceeding further, i.e. prior to ITLC analysis, HPLC analysis (see Example 12) confirmation that pH is in the range of 6.0 to 7.5, testing in a receptor binding assay, for example as described in Example 11. Neutrokine-alpha complex prepared as described above was shown to be stable for 8 hours at room temperature. Storage of the Neutrokine-alpha complex at 2-8° C. would be expected to extend its shelf life.

Optimally, therapeutic drug product will meet or exceed the following specifications: 10% (or less) free ⁹⁰Y as determined by ITLC, 10% (or less) protein aggregates relative to trimer as determined by HPLC (see Example 12), pH 6.5-7.5, with at least 75% specific binding in a receptor binding assay (see polystyrene bead assay in Example 11).

¹¹¹In Labeling Protocol

In a 10 cc vial from West (pressure washed with water for injection) combine:

1.8 milliliters of sodium acetate (NaOAc, 275 mM) with 0.4 milliliters of sodium bicarbonate (NaHCO₃, 377 mM).

10 mCi of sterile ¹¹¹In (MDS Nordion, Ontario, Canada) in 0.08 mM InCl₃

1 milliliter of Neutrokine-alpha conjugate as prepared in Examples 4-5 (2 mg/mL).

Mix the vial by gentle inversion and react for 45 minutes at room temperature. Stop the reaction by adding diluent (140 mM NaCi, 2 mM DPTA, and 10% sodium ascorbate) to a final volume of 9.95 milliliters. Let the mixture stand for five minutes prior to proceeding further, i.e. prior to ITLC analysis, HPLC analysis (see Example 12) confirmation that pH is in the range of 6.0 to 7.5, testing in a receptor binding assay, for example as described in Example 11. Neutrokine-alpha complex prepared as described above was shown to be stable for 8 hours at room temperature. Storage of the Neutrokine-alpha complex at 2-8° C. would be expected to extend its shelf life.

In a preferred embodiment, therapeutic drug product will meet or exceed the following specifications: 10% (or less) free ¹¹¹In as determined by ITLC, 10% (or less) protein aggregates relative to trimer as determined by HPLC (see Example 12), pH 6.5-7.5, with at least 75% specific binding in a receptor binding assay (e.g., see polystyrene bead assay in Example 11).

Reagents for the labeling reaction may be provided in kit form. The kit would contain one or more of the following: (a) a vial pre-filled with reaction buffer (e.g., 275 mM sodium acetate, 377 mM sodium bicarbonate in a ratio of 4:1, respectively), (b) a vial pre-filled with diluent (e.g., 140 mM NaCl, 2 mM DPTA, and 10% sodium ascorbate), (c) a vial containing Neutrokine-alpha conjugate (e.g., 2 milligrams per milliliter), (d) an empty reaction vial and (e) a vial containing the radionuclide.

In a specific embodiment, a kit of the invention contains vials (a), (b) and (c). In this case, where no empty reaction vial is included in the kit, one may be provided separately; alternatively, the vial containing the Neutrokine-alpha complex or the reaction buffer may be used as the reaction vial. The reaction vial should be large enough to contain the full volume of the final drug product, (e.g., at least lOcc in this example).

### Example 8

### Spect Imaging

The test dosage of radioactivity for these studies is 10 mCi of a Neutrokine-alpha complex prepared according to Example 6 or 7. The preparation of the injection material (i.e., sterilization, final calibration of dosage and loading the syringe) is done in a radiopharmacy. A Rhesus monkey is anesthetized by inhalation Metophane, and an intravenous catheter implanted for injection of the Neutrokine-alpha complex and transported to the SPECT suite. The animal is maintained under anesthesia for the duration of the procedure. The monkey's head is placed in a custom-designed animal-sized collimator for the scan and data is obtained continuously for 120 minutes following an intravenous bolus of the complex. The binding sites of Neutrokine-alpha are visualized using the SPECT data. This test is performed at approximately the peak of receptor occupancy. Following the completion of the scan the monkey is kept in a containment facility until the radioactive material is clear from the system.

Alternatively, a full body scan is performed to determine total distribution of the Neutrokine-alpha complex.

### Example 9

### Treatment of Lung Cancer Patient

A human patient having small-cell carcinoma of the right lung is infused intravenously with a sterile, pyrogen-free solution containing 4 mg of the Neutrokine-alpha complex in sterile, buffered saline, prepared as described herein, for example according to Example 6 or 7 hereof. After 5 days, the conjugate is well localized in the lung and has substantially cleared from the circulation of the patient, as seen by imaging scanning at daily intervals.

### Example 10

### Therapy of Lymphoma

A human patient suffering from lymphoma is infused intravenously with a sterile, pyrogen-free solution containing 4 mg of the Neutrokine-alpha complex in sterile, buffered saline, prepared as described herein, for example according to Example 6 or 7 hereof. After 6 days, the conjugate is well localized at the target and has substantially cleared from the circulation of the patient, as seen by imaging scanning at daily intervals.

### Example 11

### Neutrokine-Alpha Receptor or Neutrokine-Alpha Antibody Binding Assay

An in vitro direct binding assay is used to assess the ability of the radiolabeled Neutrokine-alpha (e.g., Neutrokine-alpha complex) protein to bind to one of its known cellular receptors, e.g., TACI, BCMA or BAFF-R, or to an anti-Neutrokine-alpha antibody (e.g., a Neutrokine-alpha antibody that neutralizes the ability of Neutrokine-alpha to bind to one of its cellular receptors). Exemplary antibodies that could be used in this assay are described in International Patent Application Publication Number WO03/55979, WO03/02641 (in particular, antibodies produced by the cell lines deposited in association with WO02/02641 and described on page 145 of WO02/02641) and WO03/33658 which are hereby incorporated by reference in their entirety. Purified Neutrokine-alpha receptor or anti-Neutrokine-alpha antibody is coated onto polystyrene beads, which are suspended in solution with an aliquot of radiolabeled protein. The coated beads and ligand are allowed to incubate for 30 minutes, at room temperature, with shaking on an Eppendorf thermomixer at 1400 rpm. The beads are then pelleted by centrifugation and frozen in liquid nitrogen. The tube is sectioned to separate the liquid phase from the pellets and both are counted on a gamma-well counter. Non-specific binding is assessed in parallel with beads that do not have Neutrokine-alpha receptor or anti-Neutrokine-alpha antibody bound to them. Percent specific binding is calculated as: [(total radioactivity bound to coated beads−radioactivity bound to uncoated beads)/(total radioactivity bound to coated beads)]×100%.

Alternatively, live cells that express Neutrokine-alpha receptors such as IM-9 cells may be used to assess the ability of the radiolabeled Neutrokine-alpha protein (e.g., Neutrokine-alpha complex) to bind to one of its cellular receptors. In such an assay, the Neutrokine-alpha complex is incubated with the cells for a period sufficient to allow binding of Neutrokine-alpha complex with Neutrokine-alpha receptors. The cells are washed to remove unbound or non-specifically bound Neutrokine-alpha complex. Cell-associated radioactivity is then measured.

### Example 12

### HPLC Analysis of Final Drug Product

The Neutrokine-alpha complex for therapeutic use may be analyzed for radiochemical purity aggregates and identity using an HPLC method with a size exclusion column. A sample of undiluted Neutrokine-alpha complex (as prepared in Example 6 or 7, for example) is loaded onto the size exclusion column. lOmM sodium acetate, with 140 mM NaCl is used as the mobile phase. The concentration of Neutrokine-alpha trimer in the final drug product may be determined by comparison of the results to a calibration curve for 0.15, 0.20, 0.25, and 0.30 milligram/milliliter of Neutrokine-alpha trimer standards. Radiochemical purity and identity can be established by comparing the test sample HPLC profile with the standard HPLC profile for trimeric Neutrokine-alpha protein. Identity can be established by HPLC retention time. Radiochemical purity can be established by the percentage labeled protein trimer versus the total labeled protein trimer and aggregates.

Each of the preceding examples can be repeated by substituting the generically or specifically described reactants and/or operating conditions of this invention for those used in the preceding examples.

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

What is claimed is:

1. A Neutrokine-alpha conjugate having the formula NA-(Chel)ₙ, wherein
NA is a Neutrokine-alpha protein;
Chel is said chelator; and
n is an integer from 1 to about 30;
wherein said Neutrokine-alpha protein comprises an amino acid sequence selected from the group consisting of:
(a) the amino acid sequence of amino acid residues 134 to 285 of SEQ ID NO:2;
(b) the amino acid sequence of amino acid residues n to 285 of SEQ ID NO:2, where n is an integer in the range of 2-190;
(c) the amino acid sequence of amino acid residues 1 to m of SEQ ID NO:2, where m is an integer in the range of 274-284;
(d) the amino acid sequence of amino acid residues n to m of SEQ ID NO:2, where n is an integer in the range of 2-190 and m is an integer in the range of 274-284; and
(e) an amino acid sequence which has at least 80% identity to any of the proteins described in (a), (b), (c), and (d);
wherein said Neutrokine-alpha protein binds a Neutrokine-alpha receptor.

2. The conjugate according to claim 1, wherein said Neutrokine-alpha protein comprises an amino acid sequence selected from the group consisting of:
(a) the amino acid sequence of amino acid residues n to 285 of SEQ ID NO:2, where n is an integer in the range of 2-190;
(b) the amino acid sequence of amino acid residues 1 to m of SEQ ID NO:2, where m is an integer in the range of 274-284; and
(c) the amino acid sequence of amino acid residues n to m of SEQ ID NO:2, where n is an integer in the range of 2-190 and m is an integer in the range of 274-284;
wherein said Neutrokine-alpha protein binds a Neutrokine-alpha receptor.

3. The conjugate of claim 1, wherein n is 1, 2, 3, 4, 5, or 6.

4. The conjugate according to claim 3, wherein n is 3.

5. The conjugate according to claim 3, wherein n is 1.

6. The conjugate according to claim 4, wherein said Neutrokine-alpha protein is a mature, soluble Neutrokine-alpha protein.

7. The conjugate according to claim 6, wherein said protein comprises a sequence that is at least 85% identical to amino acids 134-285 of SEQ ID NO:2.

8. The conjugate according to claim 7, wherein said Neutrokine-alpha consists of a trimer of Neutrokine-alpha monomeric subunits and wherein each subunit consists of amino acids 134-285 of SEQ ID NO:2.

9. The conjugate according to claim 8, wherein at least one or more lysine residues or N-terminal alanine residues of said Neutrokine-alpha protein forms the covalent bond with said chelator.

10. The conjugate according to claim 9, wherein at least one or more N-terminal alanine residues of said Neutrokine-alpha protein forms the covalent bond with said chelator.

11. The conjugate according to claim 1, wherein said chelator is DOTA, a DOTA derivative, or a DOTA analogue, optionally containing a linker moiety.

12. The conjugate according to claim 11, having the formula 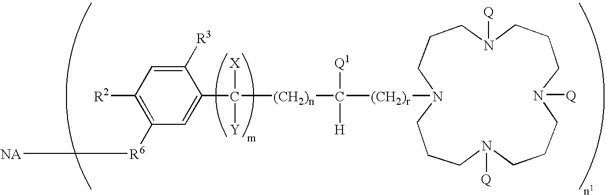
wherein
each Q is independently hydrogen or (CHR⁵)ₚCO₂R;
Q¹ is hydrogen or (CHR⁵)_{w}CO₂R;
each R independently is hydrogen, benzyl or C₁-C₄ alkyl;
with the proviso that at least two of the sum of Q and Q¹ must be other than hydrogen;
each R⁵ independently is hydrogen, C₁-C₄ alkyl or —(C₁-C₂ alkyl)phenyl;
X and Y are each independently hydrogen or may be taken with an adjacent X and Y to form an additional carbon-carbon bond;
n is 0 or 1;
n′ is 1 to 10;
m is an integer from 0 to 10 inclusive;
p is 1 or 2;
r is 0 or 1;
w is 0 or 1;
with the proviso that n is only 1 when X and/or Y form an additional carbon-carbon bond, and the sum of r and w is 0 or 1;
R² is selected from the group consisting of hydrogen, nitro, amino, isothiocyanato, semicarbazido, thiosemicarbazido, maleimido, bromoacetamido and carboxyl;
R³ is selected from the group consisting of C₁-C₄ alkoxy, —OCH₂CO₂H, hydroxy and hydrogen;
R⁶ is a group formed from a chemical group selected from the group consisting of nitro, amino, isothiocyanato, semicarbazido, thiosemicarbazido, maleimido, bromoacetamido, and carboxyl;
swith the proviso that R² and R⁶ cannot both be hydrogen but one of R² and R⁴ must be hydrogen; or a pharmaceutically acceptable salt thereof.

13. The conjugate according to claim 12, having the formula 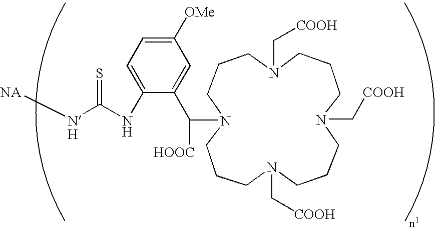
or a pharmaceutically acceptable salt thereof, wherein n¹ is 1 to 10.

14. The conjugate according to claim 11, wherein NA is a human, mature, soluble Neutrokine-alpha protein.

15. The conjugate according to claim 14, wherein said NA has a sequence that is at least 85% identical to amino acids 134-285 of SEQ ID NO:2.

16. The conjugate according to claim 15, wherein said Neutrokine-alpha consists of a trimer of Neutrokine-alpha monomeric subunits and wherein each subunit consists of amino acids 134-285 of SEQ ID NO:2.

17. The conjugate according to claim 16, wherein at least one or more lysine residues or N-terminal alanine residues of said Neutrokine-alpha protein forms the covalent bond with (Chel)ₙ.

18. The conjugate according to claim 17, wherein at least one or more N-terminal alanine residues of said Neutrokine-alpha protein forms the covalent bond with (Chel)ₙ.

19. The conjugate according to claim 18, wherein n is 1.

20. A Neutrokine-alpha conjugate having the formula: 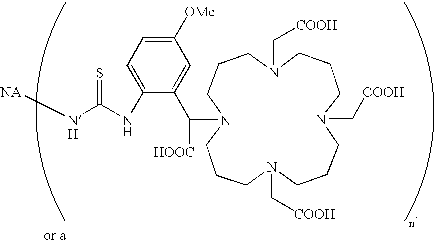
pharmaceutically acceptable salt thereof,
wherein NA is a Neutrokine-alpha protein that consists of a trimer of Neutrokine-alpha monomeric subunits wherein each subunit consists of amino acids 134-285 of SEQ ID NO:2;
n¹ is 1, 2, 3, 4, 5 or 6;
N′ is a nitrogen from the amino terminus or from a lysine residue of said Neutrokine-alpha protein.

21. A Neutrokine-alpha complex comprising a Neutrokine-alpha conjugate and a metal ion wherein said metal ion is associated with the chelator moiety of said Neutrokine-alpha conjugate.

22. The complex of claim 21, wherein said Neutrokine-alpha conjugate has the formula NA-(Chel)ₙ, wherein
NA is a Neutrokine-alpha protein;
Chel is said chelator; and
n is an integer from 1 to about 10;
wherein said Neutrokine-alpha protein comprises an amino acid sequence selected from the group consisting of:
(a) the amino acid sequence of amino acid residues n to 285 of SEQ ID NO:2, where n is an integer in the range of 2-190;
(b) the amino acid sequence of amino acid residues 1 to m of SEQ ID NO:2, where m is an integer in the range of 274-284;
(c) the amino acid sequence of amino acid residues n to m of SEQ ID NO:2, where n is an integer in the range of 2-190 and m is an integer in the range of 274-284; and
(d) an amino acid sequence which has at least 80% identity to any of the proteins described in (a), (b), and (c);
wherein said Neutrokine-alpha protein binds a Neutrokine-alpha receptor.

23. The complex according to claim 22 wherein said Neutrokine-alpha protein comprises an amino acid sequence selected from the group consisting of:
(a) the amino acid sequence of amino acid residues n to 285 of SEQ ID NO:2, where n is an integer in the range of 2-190;
(b) the amino acid sequence of amino acid residues 1 to m of SEQ ID NO:2, where m is an integer in the range of 274-284; and
(c) the amino acid sequence of amino acid residues n to m of SEQ ID NO:2, where n is an integer in the range of 2-190 and m is an integer in the range of 274-284;
wherein said Neutrokine-alpha protein binds a Neutrokine-alpha receptor.

24. The complex of claim 22, wherein n is 1, 2, 3, 4, 5, or 6.

25. The complex according to claim 24, wherein n is 3.

26. The complex according to claim 25, wherein n is 1.

27. The complex according to claim 25, wherein said Neutrokine-alpha protein is a mature, soluble Neutrokine-alpha protein.

28. The complex according to claim 27, wherein said Neutrokine-alpha protein has a sequence that is at least 85% identical to amino acids 134-285 of SEQ ID NO:2.

29. The complex according to claim 28, wherein said Neutrokine-alpha consists of amino acids 134-285 of SEQ ID NO:2.

30. The complex according to claim 29, wherein at least one or more lysine residues or N-terminal alanine residues of said Neutrokine-alpha protein forms the covalent bond with said chelator.

31. The complex according to claim 30, wherein at least one or more N-terminal alanine residues of said Neutrokine-alpha protein forms the covalent bond with said chelator.

32. The complex according to claim 22, wherein the chelator is DOTA, a DOTA derivative, or a DOTA analog, optionally containing a linker moiety.

33. The complex according to claim 32, wherein the conjugate has the formula 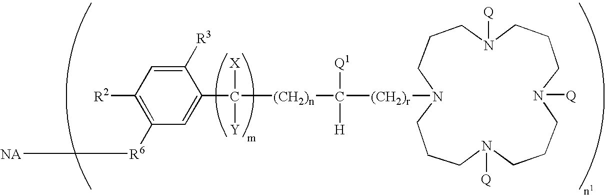
wherein
each Q is independently hydrogen or (CHR⁵)ₚCO₂R;
Q¹ is hydrogen or (CHR⁵)_{w}CO₂R;
each R independently is hydrogen, benzyl or C₁-C₄ alkyl;
with the proviso that at least two of the sum of Q and Q¹ must be other than hydrogen;
each R⁵ independently is hydrogen, C₁-C₄ alkyl or —(C₁-C₂ alkyl)phenyl;
X and Y are each independently hydrogen or may be taken with an adjacent X and Y to form an additional carbon-carbon bond;
n is 0 or 1;
n′ is 1 to 10;
m is an integer from 0 to 10 inclusive;
p is 1 or 2;
r is 0 or 1;
w is 0 or 1;
with the proviso that n is only 1 when X and/or Y form an additional carbon-carbon bond, and the sum of r and w is 0 or 1;
R² is selected from the group consisting of hydrogen, nitro, amino, isothiocyanato, semicarbazido, thiosemicarbazido, maleimido, bromoacetamido and carboxyl;
R³ is selected from the group consisting of C₁-C₄ alkoxy, —OCH₂CO₂H, hydroxy and hydrogen;
R⁶ is a functional group formed from a group selected from the group consisting of nitro, amino, isothiocyanato, semicarbazido, thiosemicarbazido, maleimido, bromoacetamido and carboxyl;
with the proviso that R² and R⁶ cannot both be hydrogen but one of R² and R⁴ must be hydrogen; or a pharmaceutically acceptable salt thereof.

34. The complex according to claim 33, wherein the conjugate has the formula 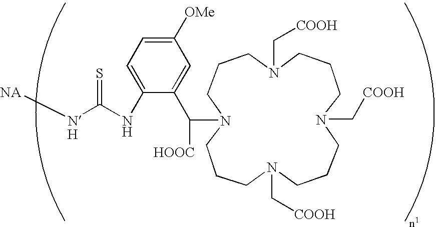
or a pharmaceutically acceptable salt thereof, wherein n¹ is 1 to 10.

35. The complex according to claim 34, wherein NA is a human, mature, soluble Neutrokine-alpha protein.

36. The complex according to claim 35, wherein said NA has a sequence that is at least 85% identical to amino acids 134-285 of SEQ ID NO:2.

37. The complex according to claim 36, wherein said Neutrokine-alpha consists of a trimer of Neutrokine-alpha monomeric subunits and wherein each subunit consists of amino acids 134-285 of SEQ ID NO:2.

38. The complex according to claim 37, wherein at least one or more lysine residues or N-terminal alanine residues of said Neutrokine-alpha protein forms the covalent bond with said chelator.

39. The complex according to claim 38, wherein at least one or more N-terminal alanine residues of said Neutrokine-alpha protein forms the covalent bond with said chelator.

40. A Neutrokine-alpha complex comprising a Neutrokine-alpha conjugate and a metal ion wherein said metal ion is associated with the chelator moiety of said Neutrokine-alpha conjugate, wherein said conjugate has the formula 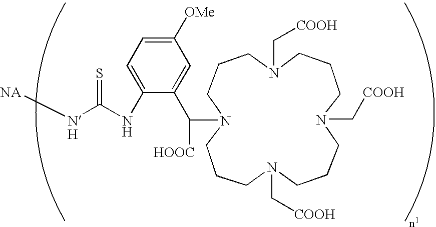
or a pharmaceutically acceptable salt thereof,
wherein NA is a Neutrokine-alpha protein that consists of a trimer of Neutrokine-alpha monomeric subunits wherein each subunit consists of amino acids 134-285 of SEQ ID NO:2;
n¹ is 1, 2, 3, 4, 5 or 6;
N′ is a nitrogen from the amino terminus or from a lysine residue of said Neutrokine-alpha protein.

41. The complex according to claim 40, wherein said metal ion is selected from the group consisting of ⁹⁰Y, ¹¹¹In, ¹⁷⁷Lu, ¹⁶⁶Ho, ²¹⁵Bi, and ²²⁵Ac.

42. The complex according to claim 41, wherein said metal ion is ⁹⁰ Y.

43. The complex according to claim 41, wherein said metal ion is ¹¹¹In.

44. The complex according to claim 41, wherein said metal ion is ¹⁷⁷Lu.

45. The complex according to claim 41, wherein said metal ion is ¹⁶⁶Ho.

46. The complex according to claim 41, wherein said metal ion is ²¹⁵Bi.

47. The complex according to claim 41, wherein said metal ion is ²²⁵Ac.

48. A composition comprising a Neutrokine-alpha conjugate according to claim 1 and a suitable carrier.

49. A composition comprising a Neutrokine-alpha conjugate according to claim 20 and a pharmaceutically acceptable carrier.

50. The composition according to claim 49, wherein said carrier is sterile water.

51. The composition according to claim 50 further comprising a buffer.

52. The composition according to claim 51 wherein said buffer is an acetate buffer having a concentration of from about 10 mM to about 200 mM.

53. The composition according to claim 49 further comprising a metal ion selected from the group consisting of ⁹⁰Y, ¹¹¹In, ¹⁷⁷Lu, ¹⁶⁶Ho, ²¹⁵Bi, and ²²⁵Ac.

54. A composition comprising a Neutrokine-alpha complex according to claim 22 and a suitable carrier.

55. A composition comprising a Neutrokine-alpha complex according to claim 40 and a pharmaceutically acceptable carrier.

56. The composition according to claim 55 wherein said carrier is sterile water.

57. The composition according to claim 56 further comprising a buffer.

58. The composition according to claim 57 wherein said buffer is an acetate buffer having a concentration of from about 10 mM to about 200 mM.

59. A method of preparing the conjugate of claim 1 comprising reacting a Neutrokine-alpha protein with a chelator.

60. The method according to claim 59, wherein said chelator is an activated chelator.

61. The method according to claim 60, wherein said activated chelator is a DOTA derivative or a DOTA analogue.

62. The method according to claim 61, wherein said chelator has the formula activated chelator having the formula: 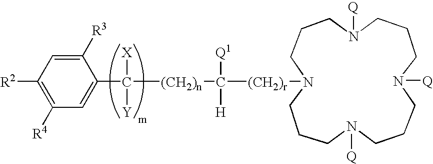
wherein
each Q is independently hydrogen or (CHR⁵)ₚCO₂R, preferably —CH₂—CO₂R, more preferably —CH₂CO₂H;
Q¹ is hydrogen or (CHR⁵)_{w}CO₂R, preferably CO₂R, more preferably COOH;
each R independently is hydrogen, benzyl or C₁-C₄ alkyl; preferably hydrogen or C₁-C₄ alkyl, more preferably hydrogen;
with the proviso that at least two of the sum of Q and Q¹ must be other than hydrogen;
each R⁵ independently is hydrogen, C₁-C₄ alkyl or —(C₁-C₂ alkyl)phenyl;
X and Y are each independently hydrogen or may be taken with an adjacent X and Y to form an additional carbon-carbon bond;
n is 0 or 1; preferably 0
m is an integer from 0 to 10 inclusive, preferably 0 to 1, more preferably 0;
p is 1 or 2, preferably 1;
r is 0 or 1, preferably 0;
w is 0 or 1, preferably 0;
with the proviso that n is only 1 when X and/or Y form an additional carbon-carbon bond, and the sum of r and w is 0 or 1;
R² is selected from the group consisting of hydrogen, nitro, amino, isothiocyanato, semicarbazido, thiosemicarbazido, maleimido, bromoacetamido and carboxyl, preferably hydrogen or isothiocyanato;
R³ is selected from the group consisting of C₁-C₄ alkoxy, —OCH₂CO₂H, hydroxy and hydrogen, preferably C₁-C₄ alkoxy, more preferably methoxy;
R⁴ is selected from the group consisting of hydrogen, nitro, amino, isothiocyanato, semicarbazido, thiosemicarbazido, maleimido, bromoacetamido and carboxyl, preferably hydrogen or isothiocyanato; with the proviso that R² and R⁴ cannot both be hydrogen but one of R² and R⁴ must be hydrogen; or a pharmaceutically acceptable salt thereof.

63. The method according to claim 62, wherein said activated chelator is α-(5-isothiocyanato-2-methoxyphenyl)-1,4,7,10-tetraazacyclododecane-1,4,7,10-tetraacetic acid.

64. The method according to claim 63, wherein the Neutrokine-alpha protein is a human, mature, soluble Neutrokine-alpha protein.

65. The method according to claim 64, wherein the Neutrokine-alpha protein has a sequence that is at least 85% identical to amino acids 134-285 of SEQ ID NO:2.

66. The method according to claim 65, wherein said Neutrokine-alpha consists of a trimer of Neutrokine-alpha monomeric subunits and wherein each subunit consists of amino acids 134-285 of SEQ ID NO:2.

67. The method according to claim 66, wherein at least one or more lysine residues or N-terminal alanine residues of said Neutrokine-alpha protein forms the covalent bond with the chelator.

68. The conjugate according to claim 67, wherein at least one or more N-terminal alanine residues of said Neutrokine-alpha protein forms the covalent bond with said chelator.

69. The method according to claim 68, comprising mixing, agitating, or preparing a solution comprising said Neutrokine-alpha protein and said chelator at a temperature of about 0° C. to about 50° C. for about 0.5 hours to about 10 hours, wherein said solution has a pH of about 8.0 to about 9.0.

70. The method according to claim 69, wherein said mixing, agitating, or preparing occurs at a temperature of about 20° C. to about 30° C. for about 3 hours to about 5 hours.

71. The method according to claim 70, further comprising adding a quenching agent after from about 3 hours to about 5 hours.

72. The method according to claim 71, wherein said pH is about 8.5.

73. The method according to claim 72, wherein the molar ratio of chelator to chelator bonding sites in the Neutrokine-alpha protein is from about 10:1 to about 12:1.

74. The method according to claim 73, wherein said quenching agent is glycine or glycine hydrochloride.

75. The method according to claim 74, wherein said solution further comprises citrate buffer and HEPES.

76. The method according to claim 75, wherein the concentration of said Neutrokine-alpha protein is from about 1.5 to about 3.0 mg/mL, and wherein the molar ratio of said chelator to chelator bonding sites in the Neutrokine-alpha protein is from about 10:1 to about 12:1.

77. The method according to claim 76, further comprising purifying said Neutrokine-alpha conjugate.

78. The method according to claim 77, wherein said purifying comprises a diafiltration method.

79. A method of preparing a Neutrokine complex according to claim 22, comprising mixing, agitating, or preparing a solution comprising a Neutrokine-alpha conjugate and a metal ion capable of complexing with a Neutrokine-alpha conjugate;
wherein said Neutrokine-alpha conjugate having the formula NA-(Chel)ₙ, wherein
(a) NA is a Neutrokine-alpha protein;
(b) Chel is said chelator; and
(c) n is an integer from 1 to about 10;
wherein said Neutrokine-alpha protein comprises an amino acid sequence selected from the group consisting of:
(a) the amino acid sequence of amino acid residues n to 285 of SEQ ID NO:2, where n is an integer in the range of 2-190;
(b) the amino acid sequence of amino acid residues 1 to m of SEQ ID NO:2, where m is an integer in the range of 274-284;
(c) the amino acid sequence of amino acid residues n to m of SEQ ID NO:2, where n is an integer in the range of 2-190 and m is an integer in the range of 274-284; and
(d) an amino acid sequence which has at least 80% identity to any of the proteins described in (a), (b), and (c);
wherein said Neutrokine-alpha protein binds a Neutrokine-alpha receptor

80. A method of preparing the complex according to claim 40, comprising mixing, agitating, or preparing a solution comprising a Neutrokine-alpha conjugate and a metal ion capable of associating with a Neutrokine-alpha conjugate;
wherein said conjugate has the formula 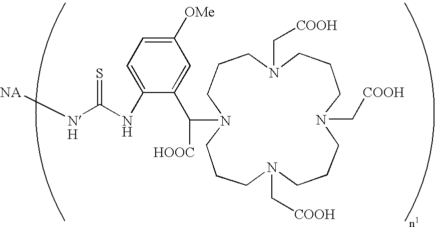
or a pharmaceutically acceptable salt thereof,
wherein NA is a Neutrokine-alpha protein that consists of a trimer of Neutrokine-alpha monomeric subunits and wherein each subunit consists of amino acids 134-285 of SEQ ID NO:2;
n¹ is 1;
N′ is a nitrogen from the amino terminus or from a lysine residue of said Neutrokine-alpha protein.

81. The method according to claim 80, further comprising removing excess metal ion.

82. The method according to claim 81, wherein said removing comprises adding a chelating agent selected from the group consisting of DPTA, EDTA, and MeO-DOTA-glycine.

83. The method according to claim 80, wherein said solution further comprises an acetate buffer having a concentration of from about 1 mM to about 20 mM and NaCl having a concentration of about 100 mM to about 200 mM.

84. The method according to claim 80, wherein the metal ion is selected from the group consisting of ⁹⁰Y, ¹¹¹In, ¹⁷⁷Lu, ¹⁶⁶Ho, ²⁵Bi, and ²²⁵Ac.

85. The method according to claim 81, wherein the solution further comprises an acetate buffer having a concentration of from about 1 mM to about 20 mM; and
further comprising allowing the solution to mix, agitate, or stand for from about 5 minutes to about 60 minutes at a temperature from about 20° C. to about 30° C.; and
adding a second solution, said second solution comprising an acetate buffer having a concentration of about 10 mM, NaCl having a concentration of about 140 mM, HSA having a concentration of about 7.5%, and DPTA having a concentration of about 1 mM, wherein said second solution has a pH of about 6.

86. A method of administering radiotherapy to a subject in need thereof, comprising administering to said subject an effective amount of a Neutrokine-alpha complex according to claim 22.

87. A method of administering radiotherapy to a subject in need thereof, comprising administering to said subject an effective amount of a Neutrokine-alpha complex according to claim 40.

88. The method of claim 87, wherein said Neutrokine-alpha complex is administered as an injectable solution.

89. The method according to claim 88, wherein said solution is administered intravenously.

90. The method according to claim 87, wherein a dosage of radioactivity from about 5 mCi to about 200 mCi is administered.

91. The method of claim 90, wherein said subject is a human.

92. The method of claim 90, wherein said subject has a B-cell mediated disease.

93. The method of claim 90, wherein said subject has a condition selected from the group consisting of non-Hodgkin's lymphoma, chronic lymphocytic leukemia, multiple myeloma, systemic lupus erythrematosus, rheumatoid arthritis, multiple sclerosis, Crohn's disease, diabetes, Wegener's granulomatous, myasthenia gravis, and asthma.

94. The method of claim 93, wherein said subject has non-Hodgkin's lymphoma.

95. A method of treating cancer comprising administering to a subject with cancer, an effective amount of a Neutrokine-alpha complex according to claim 40.

96. The method of claim 95 wherein a cell of said cancer expresses a Neutrokine-alpha receptor on its surface.

97. The method of claim 95 wherein said cancer is a B cell cancer.

98. The method of claim 97 wherein said B cell cancer is selected from the group consisting of:
(a) Non-Hodgkin's Lymphoma;
(b) Multiple Myeloma; and
(c) Chronic Lymphocytic Leukemia.

99. A method of treating an autoimmune disease or disorder comprising administering to a subject with an autoimmune disease or disorder, an effective amount of a Neutrokine-alpha complex according to claim 40.

100. The method of claim 99 wherein said autoimmune disease or disorder is selected from the group consisting of:
(a) Systemic Lupus Erythematosus;
(b) Rheumatoid Arthritis; and
(c) Sjögren's Syndrome.

101. A method of killing a cell selected from the group consisting of:
(a) a cell bearing a Neutrokine-alpha receptor; and
(b) a cell in close proximity to a cell bearing Neutrokine-alpha receptors;
wherein said method comprises contacting said cell with a composition according to claim 22 in an amount effective to kill a said cell.

102. The method of claim 101 wherein said cell is (a).

103. The method of claim 101 wherein said cell is (b).

104. The method of claim 101 wherein said cell is a lymphocyte.

105. The method of claim 104 wherein said cell is B cell.

106. The method of claim 101 wherein said cell is cancerous cell that has metastasized into the lymphatic system.

107. A method of diagnostic imaging, comprising administering a Neutrokine-alpha complex according to claim 22.

108. A kit comprising a first vial containing a Neutrokine-alpha conjugate.

109. The kit according to claim 108, wherein
said first vial contains a Neutrokine-alpha conjugate in a solution of acetate buffer (10 mM sodium acetate, 140 mM sodium chloride, pH 6.0) said conjugate having the formula 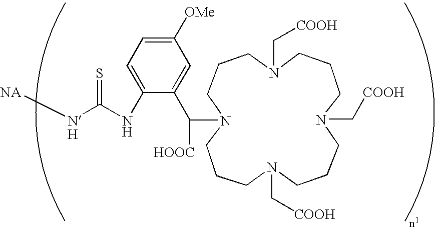
or a pharmaceutically acceptable salt thereof,
wherein NA is a Neutrokine-alpha protein that consists of a trimer of Neutrokine-alpha monomeric subunits wherein each subunit consists of amino acids 134-285 of SEQ ID NO:2;
n¹ is 1, 2, 3, 4, 5 or 6; and
N′ is a nitrogen from the amino terminus or from a lysine residue of said Neutrokine-alpha protein.

110. The kit of claim 108 comprising
(a) a second vial containing a radionuclide; and
(b) a third vial containing a buffer solution.

111. The kit according to claim 110 which further comprises a fourth vial wherein said vial is empty.

112. The kit according to claim 110, wherein said first vial contains a Neutrokine-alpha conjugate in a solution of acetate buffer (10 mM sodium acetate, 140 mM sodium chloride, pH 6.0) said conjugate having the formula 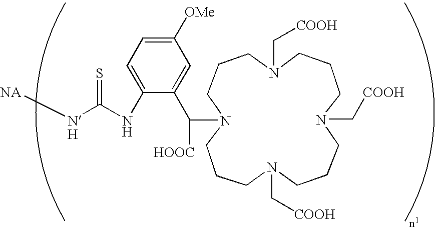
or a pharmaceutically acceptable salt thereof,
wherein NA is a Neutrokine-alpha protein that consists of a trimer of Neutrokine-alpha monomeric subunits wherein each subunit consists of amino acids 134-285 of SEQ ID NO:2;
n¹ is 1, 2, 3, 4, 5 or 6; and
N′ is a nitrogen from the amino terminus or from a lysine residue of said Neutrokine-alpha protein;
said second vial contains a radionuclide selected from the group consisting of ⁹⁰Y, ¹¹¹In, ¹⁷⁷Lu, ¹⁶⁶Ho ²¹Bi, and ²²Ac; and
said third vial contains a buffer solution, wherein said buffer solutuion is an acetate buffer solution having a concentration in the range of about 50 mM to about 300 mM.

113. The kit of claim 108 comprising
(c) a second vial containing buffer solution; and
(d) a third vial containing a diluent.

114. The kit according to claim 113 which further comprises a fourth vial wherein said vial is empty.

115. The kit according to claim 113, wherein said first vial contains a Neutrokine-alpha conjugate in a solution comprising acetate buffer having a concentration of from about 1 mM to about 20 mM and NaCl having a concentration of about 100 mM to about 200 mM and having a pH of about 6, said conjugate having the formula 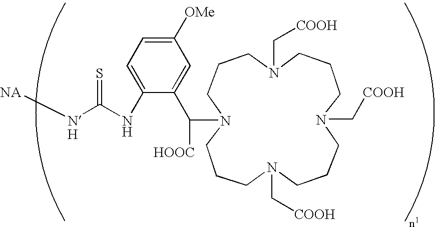
or a pharmaceutically acceptable salt thereof,
wherein NA is a Neutrokine-alpha protein that consists of a trimer of Neutrokine-alpha monomeric subunits wherein each subunit consists of amino acids 134-285 of SEQ ID NO:2;
n¹ is 1, 2, 3, 4, 5 or 6; and
N′ is a nitrogen from the amino terminus or from a lysine residue of said Neutrokine-alpha protein;
wherein said buffer solution in said second vial comprises sodium acetate in a concentration range of about 50 mM to about 300 mM and sodium bicarbonate in a concentration range of about 50 mM to about 300 mM; and
said diluent in said third vial comprises about 100 mM to about 200 mM NaCl, about 1 mM to about 10 mM DPTA, and about from 7% to about 10% sodium ascorbate

116. The kit according to claim 115, wherein said solution in said first vial comprises 10 mM sodium acetate, 140 mM NaCl.

117. The kit according to claim 115, wherein said buffer solution in said second vial comprises 220 mM sodium acetate and 75 mM sodium bicarbonate.

118. The kit according to claim 115, wherein said diluent in said third vial comprises 140 mM NaCl, 2 mM DPTA and 10% sodium ascorbate.

119. The method according to claim 80, further comprising adding to the solution a second solution to form a mixture, said second solution comprising about 220 mM sodium acetate and about 75 mM sodium bicarbonate and allowing the mixture of the solution containing the Neutrokine-alpha conjugate, the metal ion, and the second solution to mix, agitate, or stand for about 5 minutes to about 60 minutes at a temperature from about 20° C. to about 30° C.

120. The method according to claim 113, further comprising adding, after the mixture has mixed, agitated, or stood, a third solution comprising about 140 mM NaCl, about 2 mM DPTA, and about 10% sodium ascorbate.

121. A composition comprising a Neutrokine-alpha conjugate according to claim 20 and a suitable carrier.

122. A composition comprising a Neutrokine-alpha complex according to claim 40 and a suitable carrier.

123. A method of diagnostic imaging, comprising administering a Neutrokine-alpha complex according to claim 40.
